(19) United States
(12) Patent Application Publication
(10) Pub. No.: US 2017/0309831 A1
(43) Pub. Date: Oct. 26, 2017

(54) COMPOSITION FOR ORGANIC OPTOELECTRIC DEVICE AND ORGANIC OPTOELECTRIC DEVICE AND DISPLAY DEVICE

(71) Applicant: SAMSUNG SDI CO., LTD., Yongin-si (KR)

(72) Inventors: Changwoo KIM, Suwon-si (KR); Hanill LEE, Suwon-si (KR); Hyung Sun KIM, Suwon-si (KR); Dong Wan RYU, Suwon-si (KR); Eun Sun YU, Suwon-si (KR); Sung-Hyun JUNG, Suwon-si (KR); Ho Kuk JUNG, Suwon-si (KR)

(21) Appl. No.: 15/419,082

(22) Filed: Jan. 30, 2017

(30) Foreign Application Priority Data
Apr. 21, 2016 (KR) 10-2016-0048868

(51) Int. Cl. H01L 51/00; C09K 11/02; C09K 11/06; C07D 251/24; C07D 487/04; H01L 51/50

(52) U.S. Cl. CPC H01L 51/0072; C07D 487/04; C09K 11/025; C09K 11/06; H01L 51/0067; H01L 51/0054; C07D 251/24; H01L 51/0085; C09K 2211/1007; C09K 2211/1029; H01L 51/5016; H01L 2251/5384; C09K 2211/185

(57) ABSTRACT

Disclosed are a composition for an organic optoelectric device including at least one of a first host compound represented by a combination of Chemical Formula 1 and Chemical Formula 2, and at least one of a second host compound represented by Chemical Formula 3, an organic optoelectric device including the same, and a display device. Details of Chemical Formulae 1 to 3 are the same as defined in the specification.

### CROSS-REFERENCE TO RELATED APPLICATION

Korean Patent Application No. 10-2016-0048868, filed on Apr. 21, 2016, in the Korean Intellectual Property Office, and entitled: “Composition for organic optoelectric device and organic optoelectric device and display device,” is incorporated by reference herein in its entirety.

### BACKGROUND OF THE INVENTION

### 1. Field of the Invention

A composition for an organic optoelectric device, an organic optoelectric device, and a display device are disclosed.

### 2. Description of the Related Art

An organic optoelectric device is a device that converts electrical energy into photoenergy, and vice versa.

An organic optoelectric device may be classified as follows in accordance with its driving principles. One is an optoelectric device where excitons are generated by photoenergy, separated into electrons and holes, and are transferred to different electrodes to generate electrical energy, and the other is a light emitting device where a voltage or a current is supplied to an electrode to generate photoenergy from electrical energy.

Examples of the organic optoelectric device may be an organic photoelectric device, an organic light emitting diode, an organic solar cell, and an organic photo conductor drum.

Of these, an organic light emitting diode (OLED) has recently drawn attention due to an increase in demand for flat panel displays. The organic light emitting diode converts electrical energy into light by applying current to an organic light emitting material and has a structure in which an organic layer is interposed between an anode and a cathode. Herein, the organic layer may include a light-emitting layer and optionally an auxiliary layer, and the auxiliary layer may be, for example at least one selected from a hole injection layer, a hole transport layer, an electron blocking layer, an electron transport layer, an electron injection layer, and a hole blocking layer for improving efficiency and stability of an organic light emitting diode.

Performance of an organic light emitting diode may be affected by characteristics of the organic layer, and among them, may be mainly affected by characteristics of an organic material of the organic layer.

Particularly, development for an organic material being capable of increasing hole and electron mobility and simultaneously increasing electrochemical stability is needed so that the organic light emitting diode may be applied to a large-size flat panel display.

### SUMMARY OF THE INVENTION

An embodiment provides a composition for an organic optoelectric device capable of realizing an organic optoelectric device having high efficiency and a long life-span.

Another embodiment provides an organic optoelectric device including the composition.

Yet another embodiment provides a display device including the organic optoelectric device.

According to one embodiment, a composition for an organic optoelectric device includes at least one of a first host compound represented by a combination of Chemical Formula 1 and Chemical Formula 2, and

at least one of a second host compound represented by Chemical Formula 3.

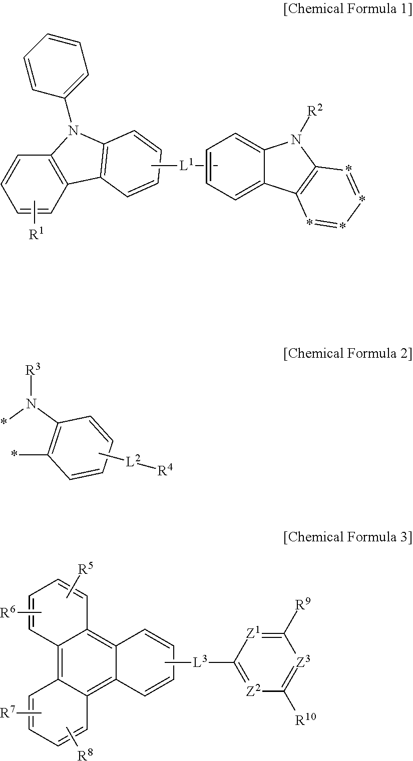

In Chemical Formulae 1 to 3,

adjacent two *'s of Chemical Formula 1 are linked with two *'s of Chemical Formula 2, and remaining *'s that are not linked with * of Chemical Formula 2 are independently CR^{a},

R¹, R⁴ and R^{a} are independently hydrogen, deuterium, a substituted or unsubstituted C6 to C30 aryl group, or a combination thereof,

R² and R³ are independently a substituted or unsubstituted C6 to C30 aryl group,

L¹ and L² are independently a single bond, or a substituted or unsubstituted phenylene group,

Z¹ to Z³ are independently CR^{b} or N,

at least one of Z¹ to Z³ is N,

R⁵ to R¹⁰ and R^{b} are independently hydrogen, deuterium, a substituted or unsubstituted C1 to C10 alkyl group, a substituted or unsubstituted C6 to C12 aryl group, a substituted or unsubstituted C2 to C12 heteroaryl group, or a combination thereof, and

L³ is a single bond, a substituted or unsubstituted phenylene group, a substituted or unsubstituted biphenylene group or a substituted or unsubstituted terphenylene group,

wherein “substituted” refers to replacement of at least one hydrogen by deuterium, a C1 to C4 alkyl group, or a C6 to C12 aryl group.

According to another embodiment, an organic optoelectric device includes an anode and a cathode facing each other and at least one organic layer between the anode and the cathode, wherein the organic layer includes the composition for an organic optoelectric device.

According to yet another embodiment, a display device including the organic optoelectric device is provided.

An organic optoelectric device having high efficiency and a long life-span may be realized.

### BRIEF DESCRIPTION OF THE DRAWINGS

FIGS. 1 and 2 are cross-sectional views showing organic light emitting diodes according to embodiments.

### DETAILED DESCRIPTION

Hereinafter, embodiments of the present invention are described in detail. However, these embodiments are exemplary, the present invention is not limited thereto and the present invention is defined by the scope of claims.

In the present specification, when a definition is not otherwise provided, “substituted” refers to one substituted with deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C6 to C30 heteroaryl group, a C1 to C20 alkoxy group, a fluoro group, a C1 to C10 trifluoroalkyl group such as a trifluoromethyl group, or a cyano group, instead of at least one hydrogen of a substituent or a compound.

In the present specification, when specific definition is not otherwise provided, “hetero” refers to one including 1 to 3 heteroatoms selected from the group consisting of N, O, S, P, and Si, and remaining carbons in one functional group.

In the present specification, when a definition is not otherwise provided, “alkyl group” refers to an aliphatic hydrocarbon group. The alkyl group may be “a saturated alkyl group” without any double bond or triple bond.

The alkyl group may be a C1 to C30 alkyl group. More specifically, the alkyl group may be a C1 to C20 alkyl group or a C1 to C10 alkyl group. For example, a C1 to C4 alkyl group may have 1 to 4 carbon atoms in an alkyl chain which may be selected from methyl, ethyl, propyl, iso-propyl, n-butyl, iso-butyl, sec-butyl, and t-butyl.

Specific examples of the alkyl group may be a methyl group, an ethyl group, a propyl group, an isopropyl group, a butyl group, an isobutyl group, a t-butyl group, a pentyl group, a hexyl group, a cyclopropyl group, a cyclobutyl group, a cyclopentyl group, a cyclohexyl group, and the like.

In the present specification, “aryl group” refers to a group including at least one hydrocarbon aromatic moiety, and

all the elements of the hydrocarbon aromatic moiety have p-orbitals which form conjugation, for example a phenyl group, a naphthyl group, and the like,

two or more hydrocarbon aromatic moieties may be linked by a sigma bond and may be, for example a biphenyl group, a terphenyl group, a quarterphenyl group, and the like, and

two or more hydrocarbon aromatic moieties are fused directly or indirectly to provide a non-aromatic fused ring. For example, it may be a fluorenyl group.

The aryl group may include a monocyclic, polycyclic or fused ring polycyclic (i.e., rings sharing adjacent pairs of carbon atoms) functional group.

For example, a “heteroaryl group” may refer to an aryl group including at least one hetero atom selected from N, O, S, P, and Si and remaining carbon. Two or more heteroaryl groups are linked by a sigma bond directly, or when the heteroaryl group includes two or more rings, the two or more rings may be fused. When the heteroaryl group is a fused ring, each ring may include 1 to 3 hetero atoms.

Specific examples of the heteroaryl group may be a pyridinyl group, a pyrimidinyl group, a pyrazinyl group, a pyridazinyl group, a triazinyl group, a quinolinyl group, an isoquinolinyl group, and the like.

More specifically, the substituted or unsubstituted C6 to C30 aryl group and/or the substituted or unsubstituted C2 to C30 heteroaryl group may be a substituted or unsubstituted phenyl group, a substituted or unsubstituted naphthyl group, a substituted or unsubstituted anthracenyl group, a substituted or unsubstituted phenanthryl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted chrysenyl group, a substituted or unsubstituted triphenylenyl group, a substituted or unsubstituted perylenyl group, a substituted or unsubstituted fluorenyl group, a substituted or unsubstituted indenyl group, a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted dibenzofuranyl group, or a substituted or unsubstituted dibenzothiophenyl group, or a combination thereof, but are not limited thereto.

In the specification, hole characteristics refer to an ability to donate an electron to form a hole when an electric field is applied and that a hole formed in the anode may be easily injected into the emission layer and transported in the emission layer due to conductive characteristics according to a highest occupied molecular orbital (HOMO) level.

In addition, electron characteristics refer to an ability to accept an electron when an electric field is applied and that electron formed in the cathode may be easily injected into the emission layer and transported in the emission layer due to conductive characteristics according to a lowest unoccupied molecular orbital (LUMO) level.

Hereinafter, a composition for an organic optoelectric device according to an embodiment is described.

A composition for an organic optoelectric device according to an embodiment includes at least two kinds of a host and a dopant, and the host includes a first host compound having relatively strong hole characteristics and a second host compound having relatively strong electron characteristics.

The first host compound is a compound having relatively strong hole transport characteristics and is represented by a combination of Chemical Formula 1 and Chemical Formula 2.

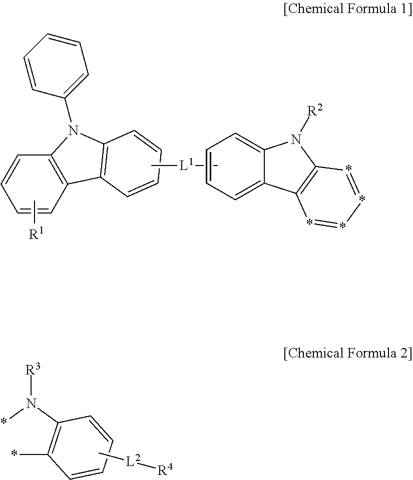

In Chemical Formulae 1 and 2,

adjacent two *'s of Chemical Formula 1 are linked with two *'s of Chemical Formula 2, and remaining *'s that are not linked with * of Chemical Formula 2 are independently CR^{a},

R¹, R⁴, and R^{a} are independently hydrogen, deuterium, a substituted or unsubstituted C6 to C30 aryl group, or a combination thereof,

R² and R³ are independently a substituted or unsubstituted C6 to C30 aryl group, and

L¹ and L² are independently a single bond, or a substituted or unsubstituted phenylene group.

The first host compound fortifies hole transport characteristics due to a carbazolyl group at the terminal end of an indolocarbazole structure, and thus luminous efficiency and life-span characteristics may be remarkably improved by increasing charge mobility and stability.

The first host compound may be, for example represented by Chemical Formula 1-A, 1-B, 1-C, 1-D, 1-E, or 1-F according to a fusing position of Chemical Formulae 1 and 2.

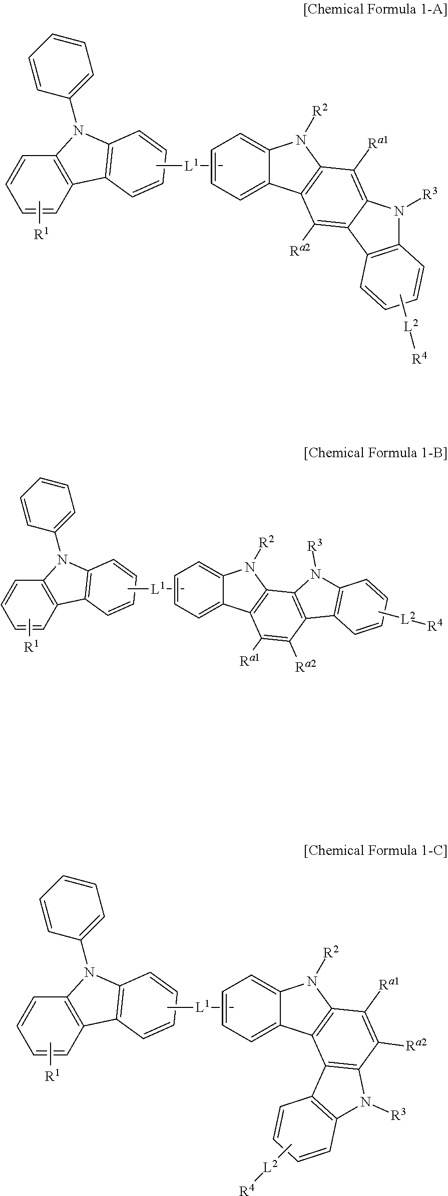

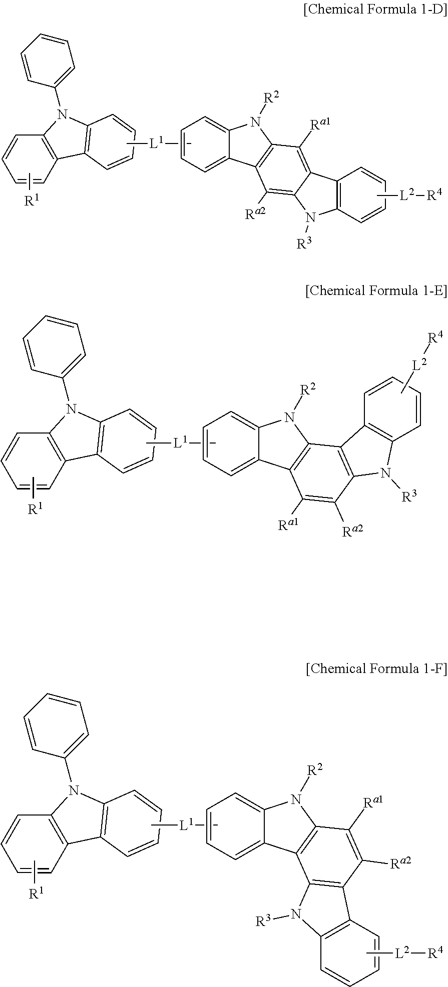

In Chemical Formulae 1-A to 1-F, R¹ to R⁴, L¹, and L² are the same as described above,

R^{a1} and R^{a2} are the same as defined in R^{a}.

The Chemical Formula 1 may be, for example represented by Chemical Formula 1-I, 1-II, 1-III, or 1-IV according to a linking point of a carbazolyl group substituting a terminal end of indolocarbazole,

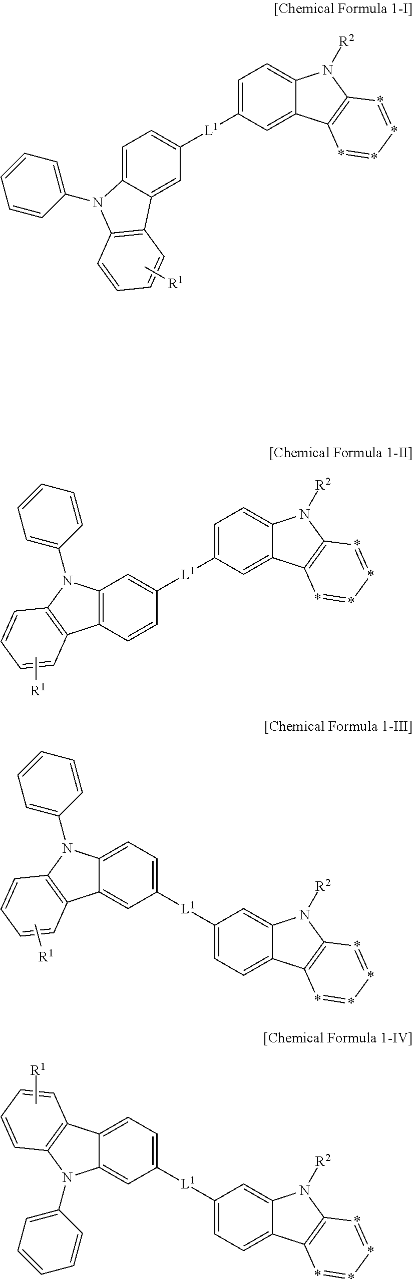

more specifically, may be represented by Chemical Formula 1-Ia, 1-Ib, 1-Ic, 1-IIa, 1-1-IIb, 1-IIc, 1-IIIb, 1-IIIc, 1-IVa, 1-IVb, or 1-IVc, and

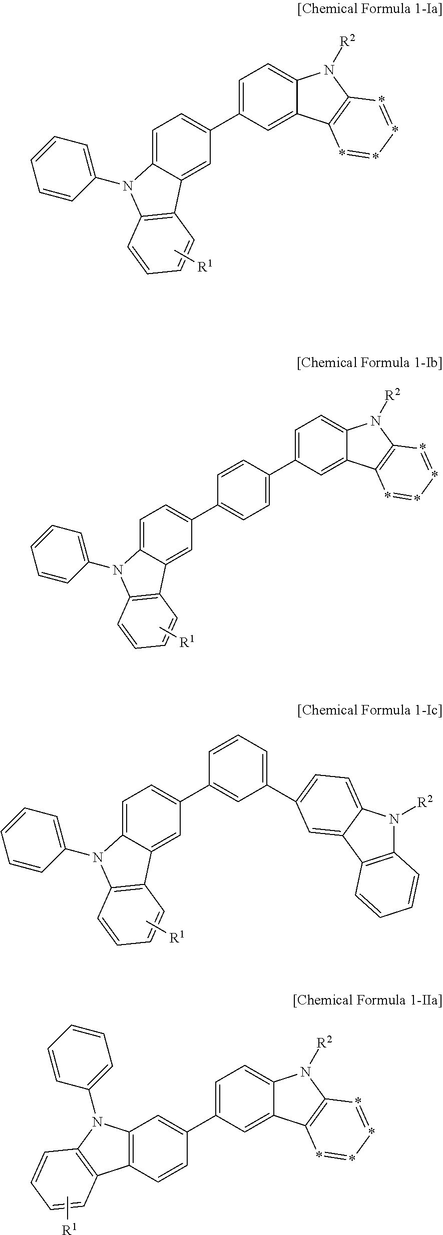

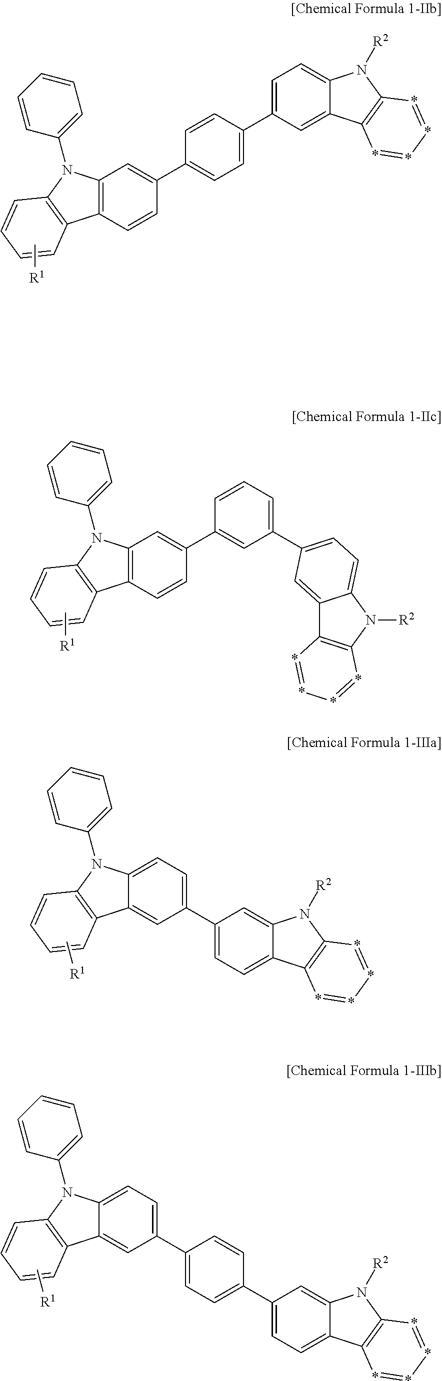

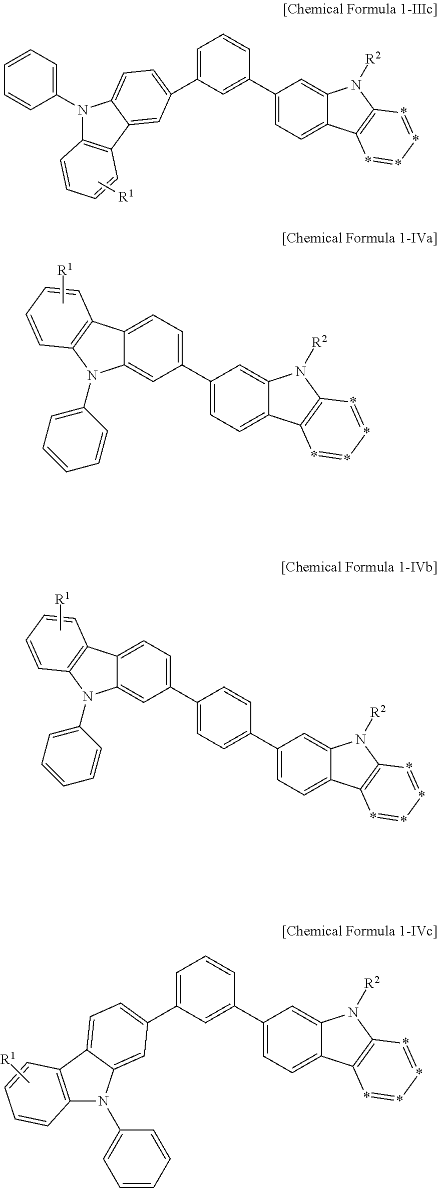

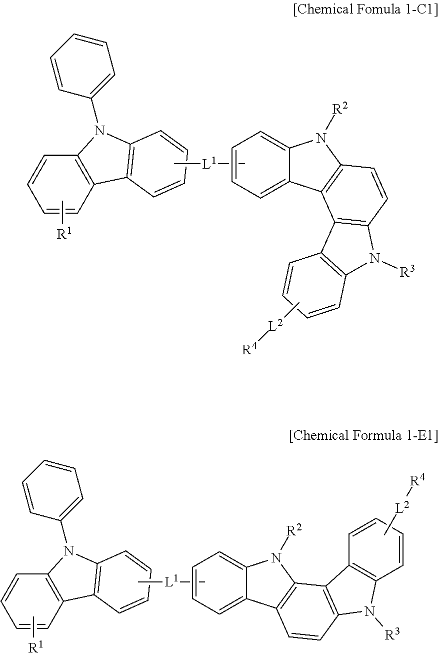

as specific examples according to an example embodiment of the present invention, it may be represented by Chemical Formula 1-Ia or 1-IIa, but is not limited thereto.

The R¹, R² and L¹ may be the same as described above.

In an example embodiment of the present invention, the R¹, R⁴ and R¹ may independently be hydrogen, deuterium, a substituted or unsubstituted C6 to C30 aryl group, or a combination thereof. Specifically, they may be hydrogen, deuterium, a substituted or unsubstituted C6 to C18 aryl group, more specifically, hydrogen, deuterium, a substituted or unsubstituted phenyl group, a substituted or unsubstituted biphenyl group, or a substituted or unsubstituted terphenyl group.

The R² and R³ are independently a substituted or unsubstituted C6 to C30 aryl group. Specifically, they may be a substituted or unsubstituted C6 to C18 aryl group, more specifically a substituted or unsubstituted phenyl group, a substituted or unsubstituted biphenyl group, a substituted or unsubstituted terphenyl group, or a substituted or unsubstituted fluorenyl group.

As specific examples according to an example embodiment of the present invention, the R¹, R⁴, and R^{a} are hydrogen, and the R² and R³ are a phenyl group, but are not limited thereto.

In an example embodiment of the present invention the L¹ and L² are independently a single bond, or a substituted or unsubstituted phenylene group. Specifically, they may be a single bond or selected from linking groups of Group I, but are not limited thereto.

[Group I]

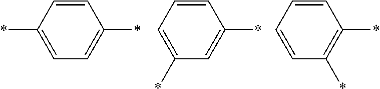

In Group I, * is a linking point.

As specific examples, the L¹ and L² may be linked in a para position or meta position.

According to examples of the present invention, the first host compound may be represented by Chemical Formula 1-C1 or Chemical Formula 1-E1.

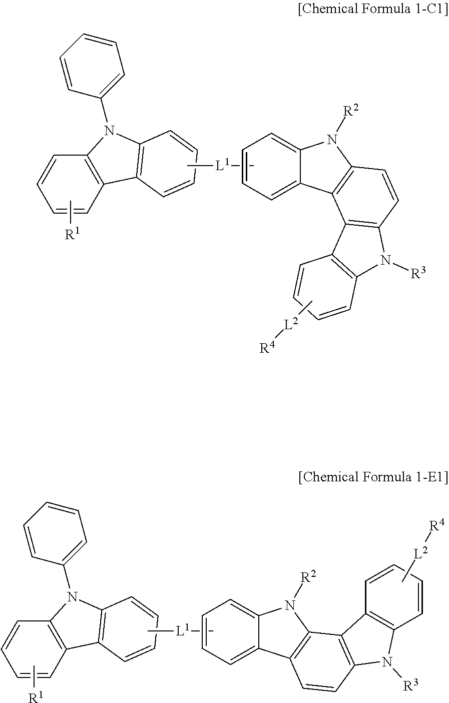

In Chemical Formulae 1-C1 and 1-E1, R¹ to R⁴, L¹ and L² are the same as described above.

The first host compound may be, for example compounds of Group 1, but is not limited thereto.

[Group 1]

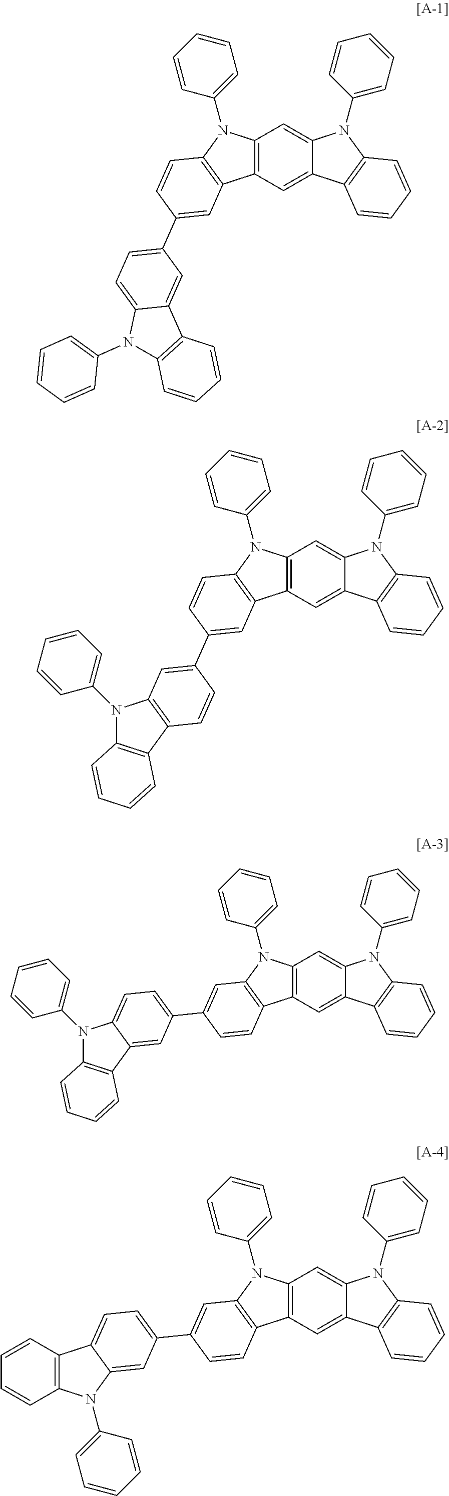 [SMILES: c1ccc(-n2c3ccccc3c3cc(-c4ccc5c(c4)c4cc6c7ccccc7n(-c7ccccc7)c6cc4n5-c4ccccc4)ccc32)cc1 ; c1ccc(-n2c3ccccc3c3ccc(-c4ccc5c(c4)c4cc6c7ccccc7n(-c7ccccc7)c6cc4n5-c4ccccc4)cc32)cc1 ; c1ccc(-n2c3ccccc3c3cc(-c4ccc5c6cc7c8ccccc8n(-c8ccccc8)c7cc6n(-c6ccccc6)c5c4)ccc32)cc1 ; c1ccc(-n2c3ccccc3c3ccc(-c4ccc5c6cc7c8ccccc8n(-c8ccccc8)c7cc6n(-c6ccccc6)c5c4)cc32)cc1]

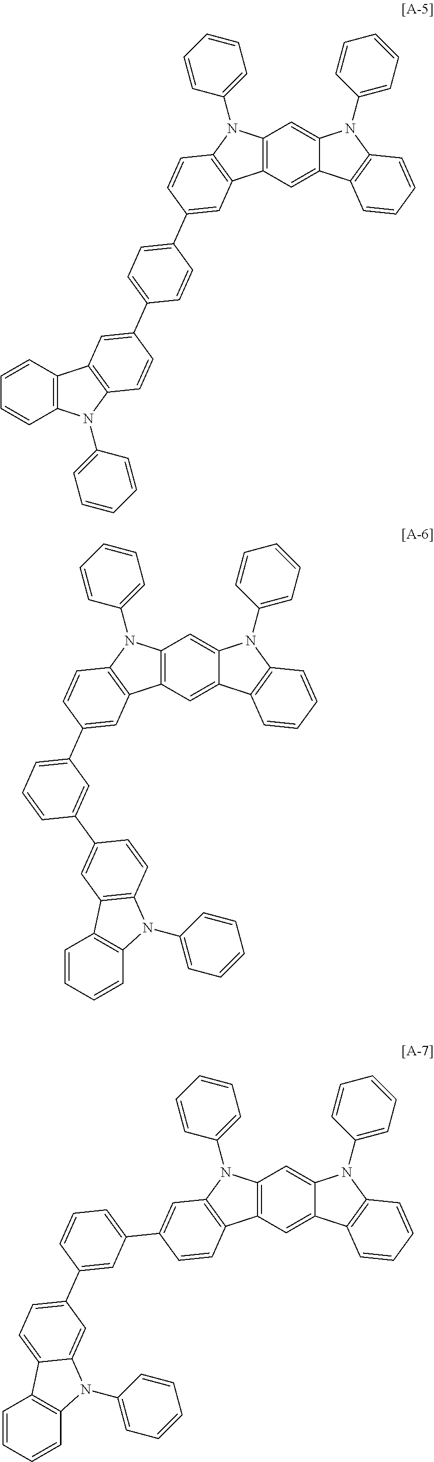

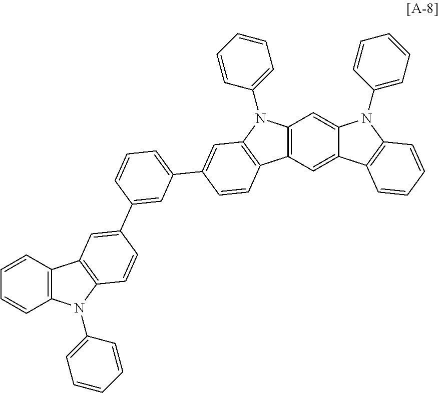 [SMILES: c1ccc(-n2c3ccccc3c3cc(-c4cccc(-c5ccc6c7cc8c9ccccc9n(-c9ccccc9)c8cc7n(-c7ccccc7)c6c5)c4)ccc32)cc1]

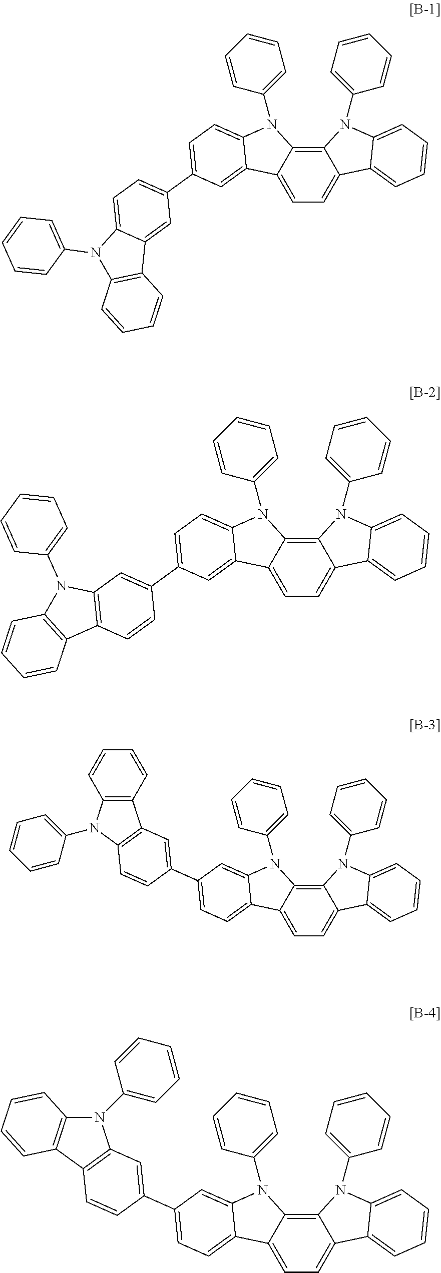 [SMILES: c1ccc(-n2c3ccccc3c3cc(-c4ccc5c(c4)c4ccc6c7ccccc7n(-c7ccccc7)c6c4n5-c4ccccc4)ccc32)cc1 ; c1ccc(-n2c3ccccc3c3ccc(-c4ccc5c(c4)c4ccc6c7ccccc7n(-c7ccccc7)c6c4n5-c4ccccc4)cc32)cc1 ; c1ccc(-n2c3ccccc3c3cc(-c4ccc5c6ccc7c8ccccc8n(-c8ccccc8)c7c6n(-c6ccccc6)c5c4)ccc32)cc1 ; c1ccc(-n2c3ccccc3c3ccc(-c4ccc5c6ccc7c8ccccc8n(-c8ccccc8)c7c6n(-c6ccccc6)c5c4)cc32)cc1]

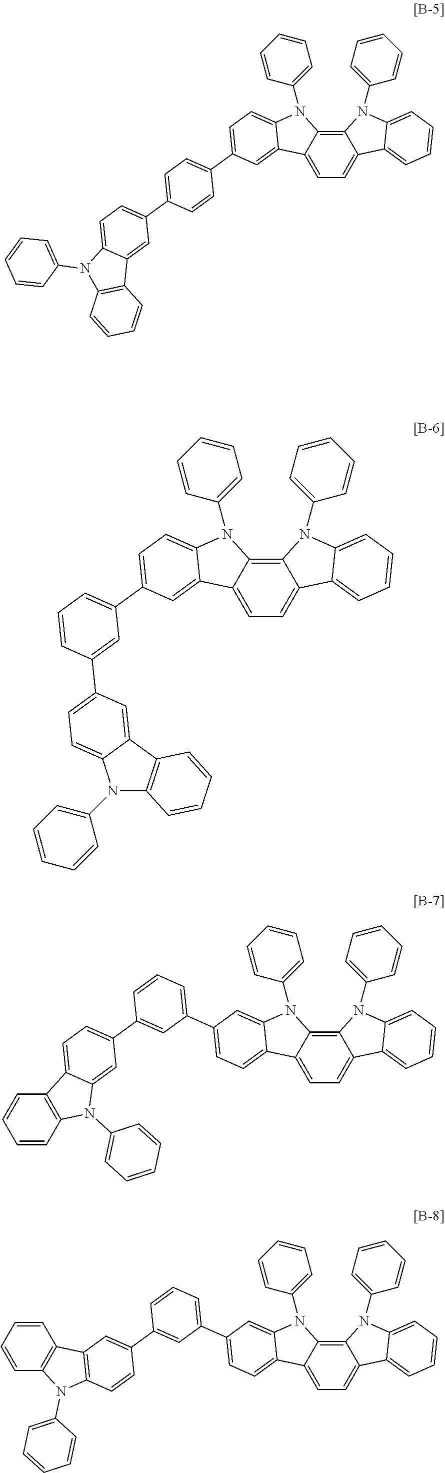 [SMILES: c1ccc(-n2c3ccccc3c3cc(-c4ccc(-c5ccc6c(c5)c5ccc7c8ccccc8n(-c8ccccc8)c7c5n6-c5ccccc5)cc4)ccc32)cc1 ; c1ccc(-n2c3ccccc3c3cc(-c4cccc(-c5ccc6c(c5)c5ccc7c8ccccc8n(-c8ccccc8)c7c5n6-c5ccccc5)c4)ccc32)cc1 ; c1ccc(-n2c3ccccc3c3ccc(-c4cccc(-c5ccc6c7ccc8c9ccccc9n(-c9ccccc9)c8c7n(-c7ccccc7)c6c5)c4)cc32)cc1 ; c1ccc(-n2c3ccccc3c3cc(-c4cccc(-c5ccc6c7ccc8c9ccccc9n(-c9ccccc9)c8c7n(-c7ccccc7)c6c5)c4)ccc32)cc1]

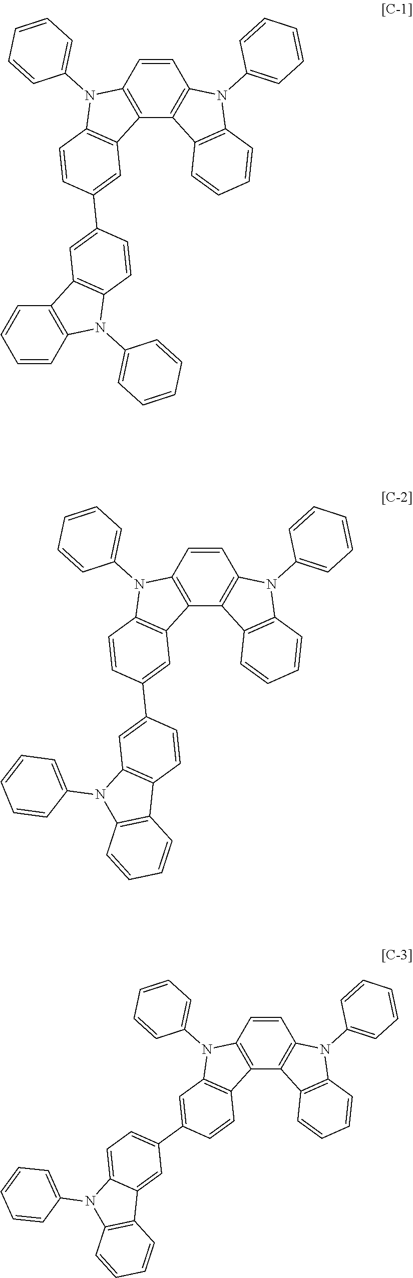 [SMILES: c1ccc(-n2c3ccccc3c3cc(-c4ccc5c(c4)c4c6c7ccccc7n(-c7ccccc7)c6ccc4n5-c4ccccc4)ccc32)cc1 ; c1ccc(-n2c3ccccc3c3ccc(-c4ccc5c(c4)c4c6c7ccccc7n(-c7ccccc7)c6ccc4n5-c4ccccc4)cc32)cc1 ; c1ccc(-n2c3ccccc3c3cc(-c4ccc5c6c7c8ccccc8n(-c8ccccc8)c7ccc6n(-c6ccccc6)c5c4)ccc32)cc1]

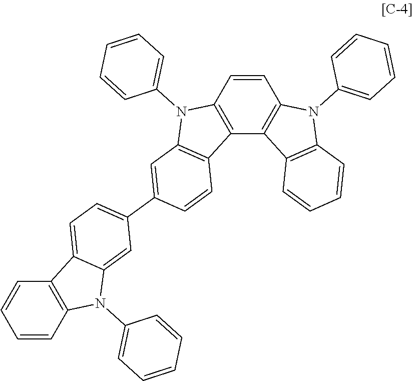 [SMILES: c1ccc(-n2c3ccccc3c3ccc(-c4ccc5c6c7c8ccccc8n(-c8ccccc8)c7ccc6n(-c6ccccc6)c5c4)cc32)cc1]

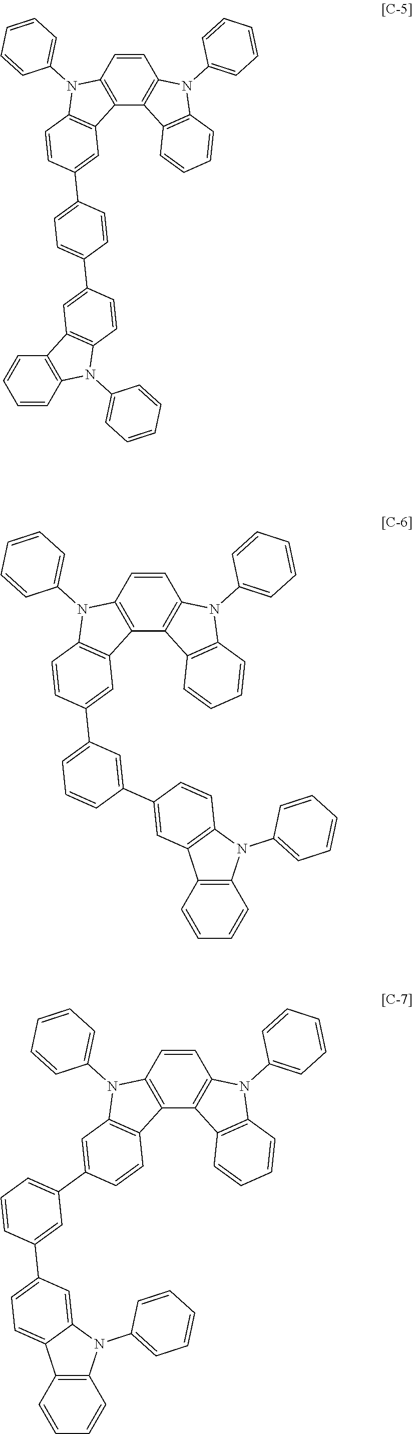 [SMILES: c1ccc(-n2c3ccccc3c3cc(-c4ccc(-c5ccc6c(c5)c5c7c8ccccc8n(-c8ccccc8)c7ccc5n6-c5ccccc5)cc4)ccc32)cc1 ; c1ccc(-n2c3ccccc3c3cc(-c4cccc(-c5ccc6c(c5)c5c7c8ccccc8n(-c8ccccc8)c7ccc5n6-c5ccccc5)c4)ccc32)cc1 ; c1ccc(-n2c3ccccc3c3ccc(-c4cccc(-c5ccc6c7c8c9ccccc9n(-c9ccccc9)c8ccc7n(-c7ccccc7)c6c5)c4)cc32)cc1]

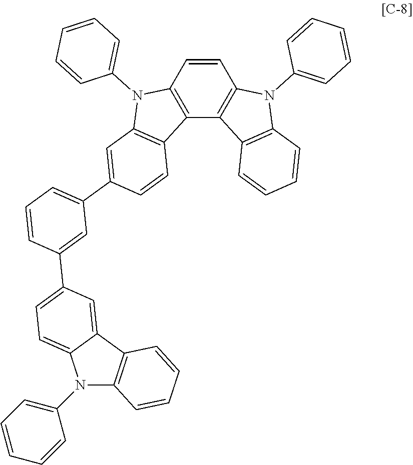 [SMILES: c1ccc(-n2c3ccccc3c3cc(-c4cccc(-c5ccc6c7c8c9ccccc9n(-c9ccccc9)c8ccc7n(-c7ccccc7)c6c5)c4)ccc32)cc1]

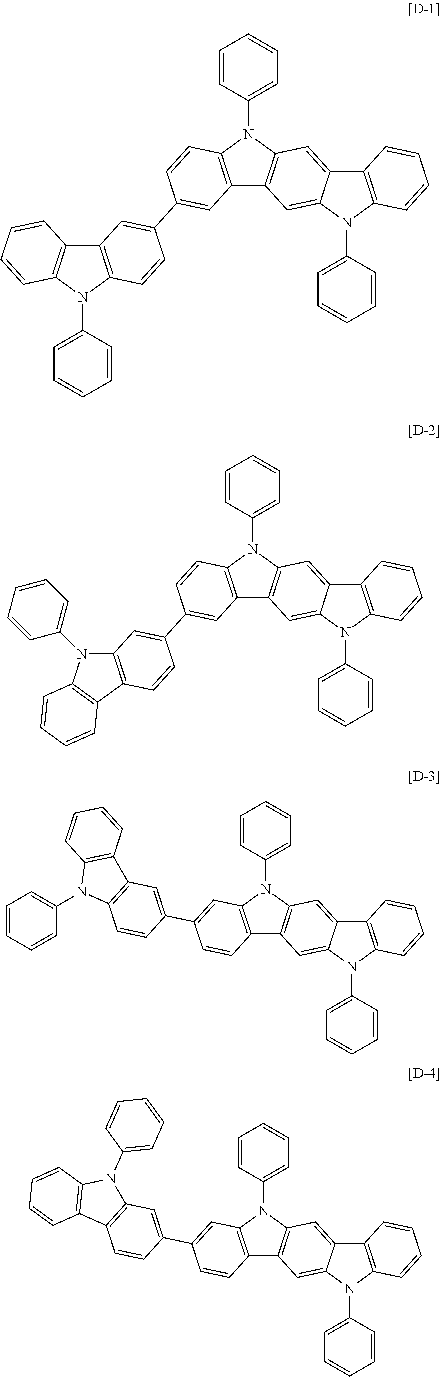 [SMILES: c1ccc(-n2c3ccccc3c3cc(-c4ccc5c(c4)c4cc6c(cc4n5-c4ccccc4)c4ccccc4n6-c4ccccc4)ccc32)cc1 ; c1ccc(-n2c3ccccc3c3ccc(-c4ccc5c(c4)c4cc6c(cc4n5-c4ccccc4)c4ccccc4n6-c4ccccc4)cc32)cc1 ; c1ccc(-n2c3ccccc3c3cc(-c4ccc5c6cc7c(cc6n(-c6ccccc6)c5c4)c4ccccc4n7-c4ccccc4)ccc32)cc1 ; c1ccc(-n2c3ccccc3c3ccc(-c4ccc5c6cc7c(cc6n(-c6ccccc6)c5c4)c4ccccc4n7-c4ccccc4)cc32)cc1]

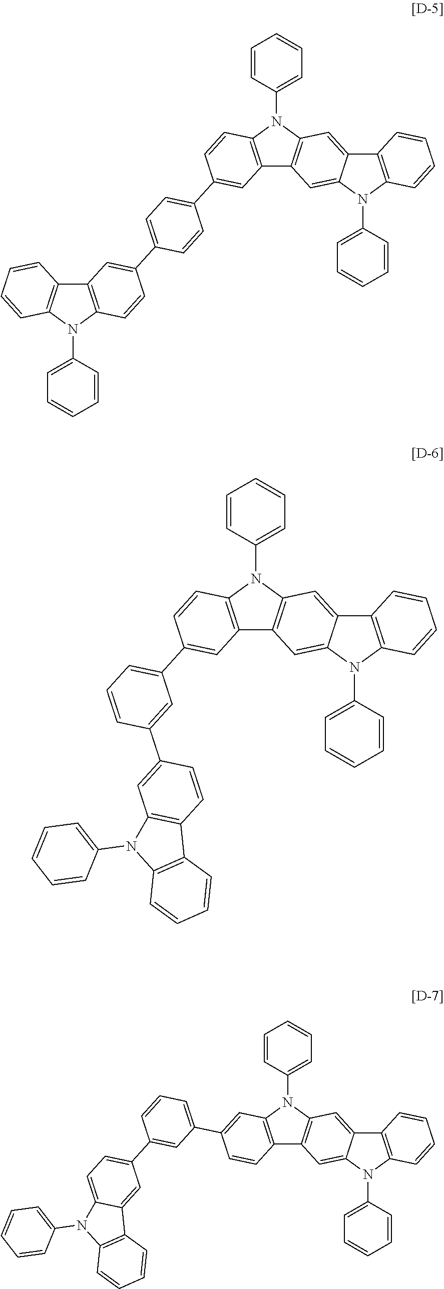 [SMILES: c1ccc(-n2c3ccccc3c3cc(-c4ccc(-c5ccc6c(c5)c5cc7c(cc5n6-c5ccccc5)c5ccccc5n7-c5ccccc5)cc4)ccc32)cc1 ; c1ccc(-n2c3ccccc3c3ccc(-c4cccc(-c5ccc6c(c5)c5cc7c(cc5n6-c5ccccc5)c5ccccc5n7-c5ccccc5)c4)cc32)cc1 ; c1ccc(-n2c3ccccc3c3cc(-c4cccc(-c5ccc6c7cc8c(cc7n(-c7ccccc7)c6c5)c5ccccc5n8-c5ccccc5)c4)ccc32)cc1]

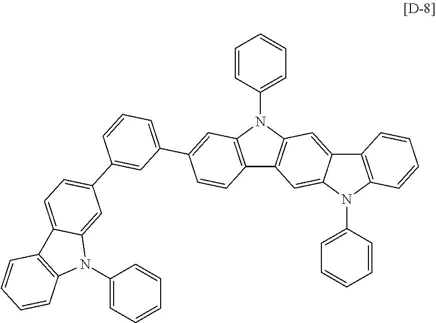 [SMILES: c1ccc(-n2c3ccccc3c3ccc(-c4cccc(-c5ccc6c7cc8c(cc7n(-c7ccccc7)c6c5)c5ccccc5n8-c5ccccc5)c4)cc32)cc1]

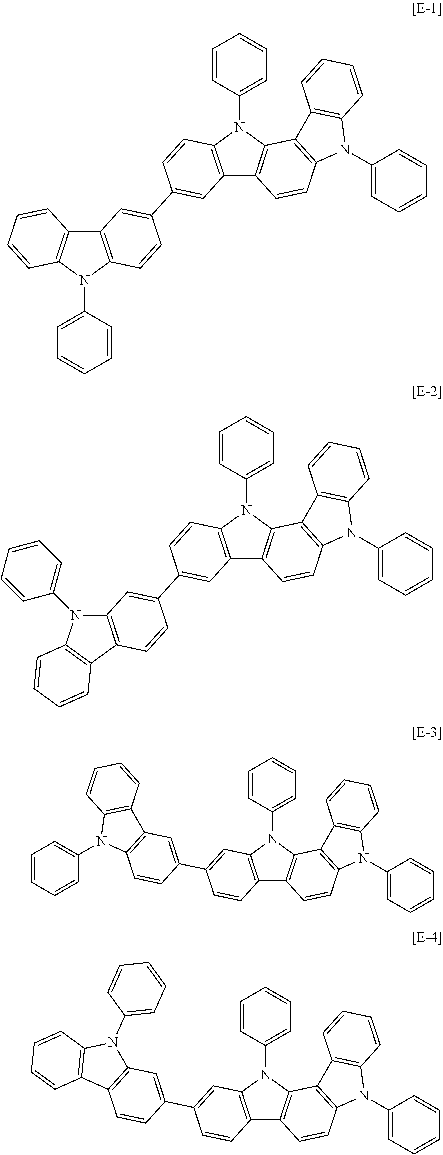 [SMILES: c1ccc(-n2c3ccccc3c3cc(-c4ccc5c(c4)c4ccc6c(c7ccccc7n6-c6ccccc6)c4n5-c4ccccc4)ccc32)cc1 ; c1ccc(-n2c3ccccc3c3ccc(-c4ccc5c(c4)c4ccc6c(c7ccccc7n6-c6ccccc6)c4n5-c4ccccc4)cc32)cc1 ; c1ccc(-n2c3ccccc3c3cc(-c4ccc5c6ccc7c(c8ccccc8n7-c7ccccc7)c6n(-c6ccccc6)c5c4)ccc32)cc1 ; c1ccc(-n2c3ccccc3c3ccc(-c4ccc5c6ccc7c(c8ccccc8n7-c7ccccc7)c6n(-c6ccccc6)c5c4)cc32)cc1]

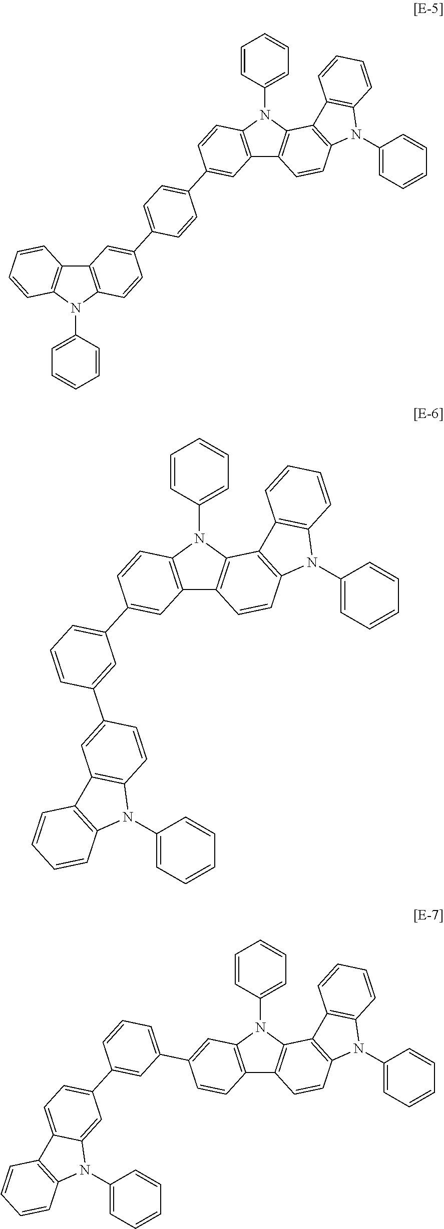 [SMILES: c1ccc(-n2c3ccccc3c3cc(-c4ccc(-c5ccc6c(c5)c5ccc7c(c8ccccc8n7-c7ccccc7)c5n6-c5ccccc5)cc4)ccc32)cc1 ; c1ccc(-n2c3ccccc3c3cc(-c4cccc(-c5ccc6c(c5)c5ccc7c(c8ccccc8n7-c7ccccc7)c5n6-c5ccccc5)c4)ccc32)cc1 ; c1ccc(-n2c3ccccc3c3ccc(-c4cccc(-c5ccc6c7ccc8c(c9ccccc9n8-c8ccccc8)c7n(-c7ccccc7)c6c5)c4)cc32)cc1]

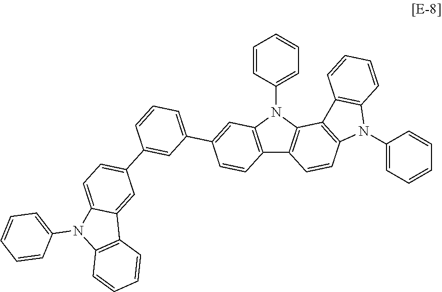 [SMILES: c1ccc(-n2c3ccccc3c3cc(-c4cccc(-c5ccc6c7ccc8c(c9ccccc9n8-c8ccccc8)c7n(-c7ccccc7)c6c5)c4)ccc32)cc1]

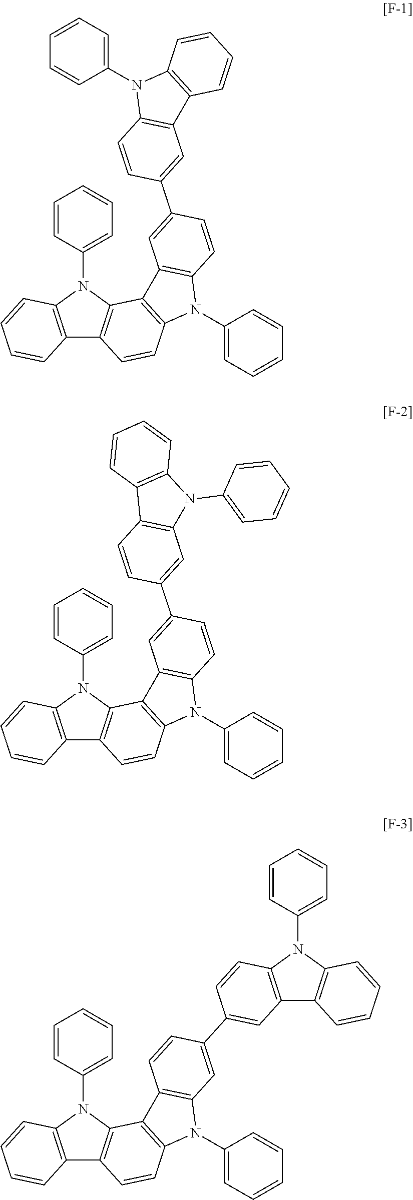 [SMILES: c1ccc(-n2c3ccccc3c3cc(-c4ccc5c(c4)c4c(ccc6c7ccccc7n(-c7ccccc7)c64)n5-c4ccccc4)ccc32)cc1 ; c1ccc(-n2c3ccccc3c3ccc(-c4ccc5c(c4)c4c(ccc6c7ccccc7n(-c7ccccc7)c64)n5-c4ccccc4)cc32)cc1 ; c1ccc(-n2c3ccccc3c3cc(-c4ccc5c6c(ccc7c8ccccc8n(-c8ccccc8)c76)n(-c6ccccc6)c5c4)ccc32)cc1]

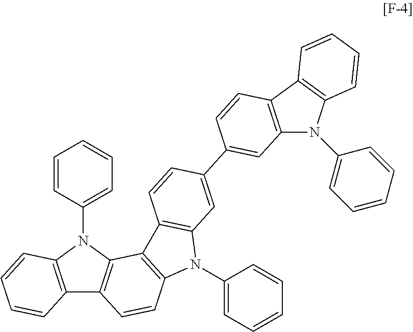 [SMILES: c1ccc(-n2c3ccccc3c3ccc(-c4ccc5c6c(ccc7c8ccccc8n(-c8ccccc8)c76)n(-c6ccccc6)c5c4)cc32)cc1]

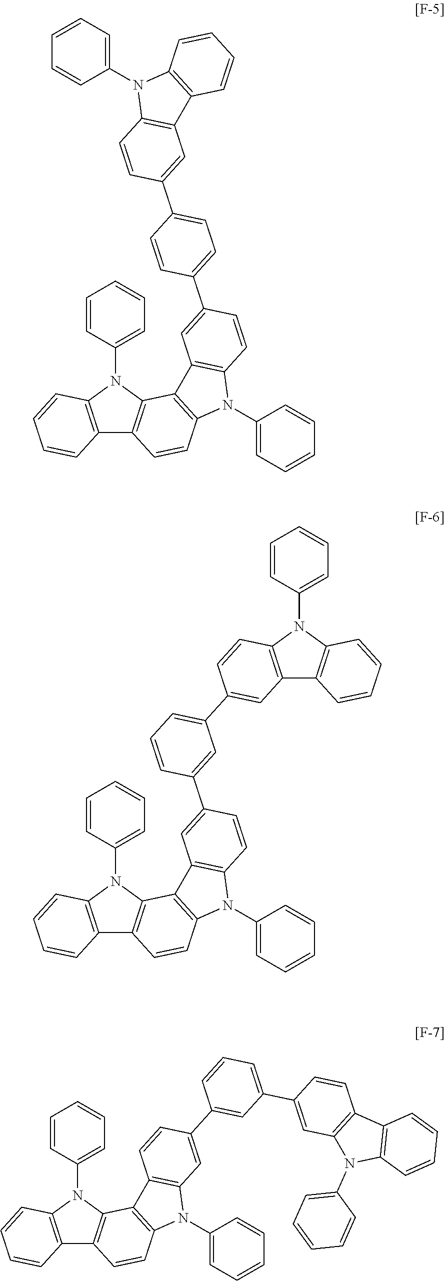 [SMILES: c1ccc(-n2c3ccccc3c3cc(-c4ccc(-c5ccc6c(c5)c5c(ccc7c8ccccc8n(-c8ccccc8)c75)n6-c5ccccc5)cc4)ccc32)cc1 ; c1ccc(-n2c3ccccc3c3cc(-c4cccc(-c5ccc6c(c5)c5c(ccc7c8ccccc8n(-c8ccccc8)c75)n6-c5ccccc5)c4)ccc32)cc1 ; c1ccc(-n2c3ccccc3c3ccc(-c4cccc(-c5ccc6c7c(ccc8c9ccccc9n(-c9ccccc9)c87)n(-c7ccccc7)c6c5)c4)cc32)cc1]

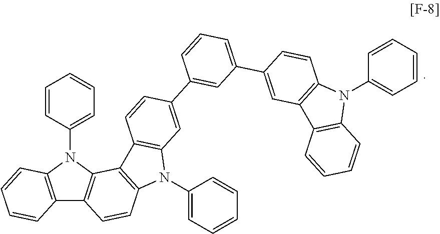 [SMILES: c1ccc(-n2c3ccccc3c3cc(-c4cccc(-c5ccc6c7c(ccc8c9ccccc9n(-c9ccccc9)c87)n(-c7ccccc7)c6c5)c4)ccc32)cc1]

The second host compound is a compound having relatively strong electron transport characteristics and is represented by a combination of Chemical Formula 1 and Chemical Formula 3.

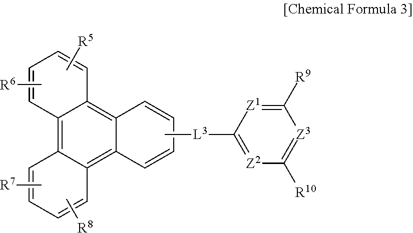

In Chemical Formula 3,

Z¹ to Z³ are independently CR^{b} or N,

at least one of Z¹ to Z³ is N,

R⁵ to R¹⁰ and R^{b} are independently hydrogen, deuterium, a substituted or unsubstituted C1 to C10 alkyl group, a substituted or unsubstituted C6 to C12 aryl group, a substituted or unsubstituted C2 to C12 heteroaryl group, or a combination thereof, and

L³ is a single bond, a substituted or unsubstituted phenylene group, a substituted or unsubstituted biphenylene group or a substituted or unsubstituted terphenylene group,

wherein “substituted” refers to replacement of at least one hydrogen by deuterium, a C1 to C4 alkyl group, or a C6 to C12 aryl group.

The second host compound includes a ring including at least one nitrogen such as a pyridinyl, pyrimidinyl, or triazinyl group in addition to a triphenylene structure and thus may have a structure easily accepting electrons when an electric field is applied thereto and accordingly, lower a driving voltage of an organic optoelectric diode manufactured by applying the first host compound.

The second host compound includes the triphenylene structure easily accepting holes and a nitrogen-containing ring moiety easily accepting electrons to form a bipolar structure, and thus may appropriately balance hole and electron flows and improve efficiency of an organic optoelectric device including the second host compound.

In an example embodiment of the present invention, two of Z¹ to Z³ of Chemical Formula 3 may be N, and specifically three may be all N. When two or more of Z¹ to Z³ are N, effect of the present invention may be realized more effectively.

The second host compound may be, for example represented by Chemical Formula 3-I or Chemical Formula 3-II according to a substitution position of the nitrogen-containing ring moiety linked with the triphenylene structure.

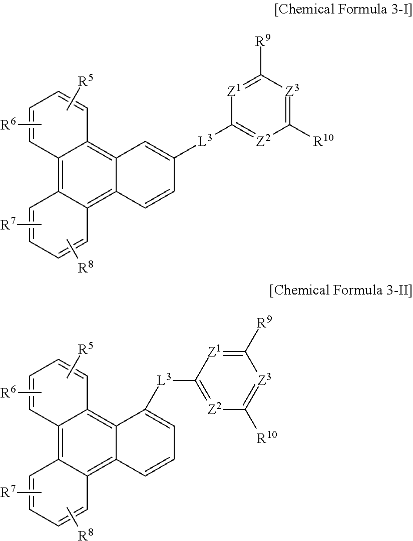

In Chemical Formulae 3-I and 3-II, Z¹ to Z³, R⁵ to R¹⁰, R^{b}, and L³ are the same as described above.

In an example embodiment of the present invention, at least one of Z¹ to Z³ may be N.

That is, a 6-membered ring consisting of Z¹ to Z³ may be a pyridinyl group, a pyrimidinyl group, or a triazinyl group. More specifically, it may be a pyrimidinyl group, or a triazinyl group.

The R⁵ to R¹⁰ and R^{b} may independently be hydrogen, deuterium, a substituted or unsubstituted C1 to C10 alkyl group, a substituted or unsubstituted C6 to C12 aryl group, a substituted or unsubstituted C2 to C12 heteroaryl group, or a combination thereof, is specifically hydrogen, deuterium, a substituted or unsubstituted C6 to C12 aryl group, or a substituted or unsubstituted C2 to C12 heteroaryl group, and is more specifically hydrogen, deuterium, a substituted or unsubstituted phenyl group, a substituted or unsubstituted biphenyl group, a substituted or unsubstituted terphenyl group, a substituted or unsubstituted pyridinyl group, a substituted or unsubstituted pyrimidinyl group, or a substituted or unsubstituted triazinyl group. As specific examples according to an example embodiment of the present invention, R⁵ to R⁸ may independently be hydrogen, or a substituted or unsubstituted phenyl group, and R⁹, R¹⁰, and R^{b} may independently be a substituted or unsubstituted phenyl group, a substituted or unsubstituted biphenyl group, a substituted or unsubstituted terphenyl group, a substituted or unsubstituted pyridinyl group, a substituted or unsubstituted pyridinyl group, a substituted or unsubstituted pyrimidinyl group, or a substituted or unsubstituted triazinyl group.

According to examples of the present invention, R⁹, R¹⁰, and R^{b} may independently be one of substituents of Group II, and as more specific examples,

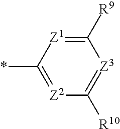

of Chemical Formula 3 may be one of substituents of Group III.

[Group II]

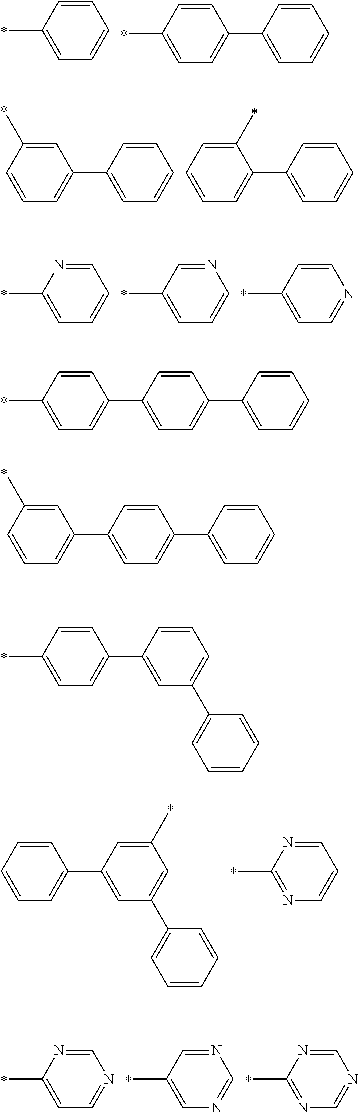

[Group III]

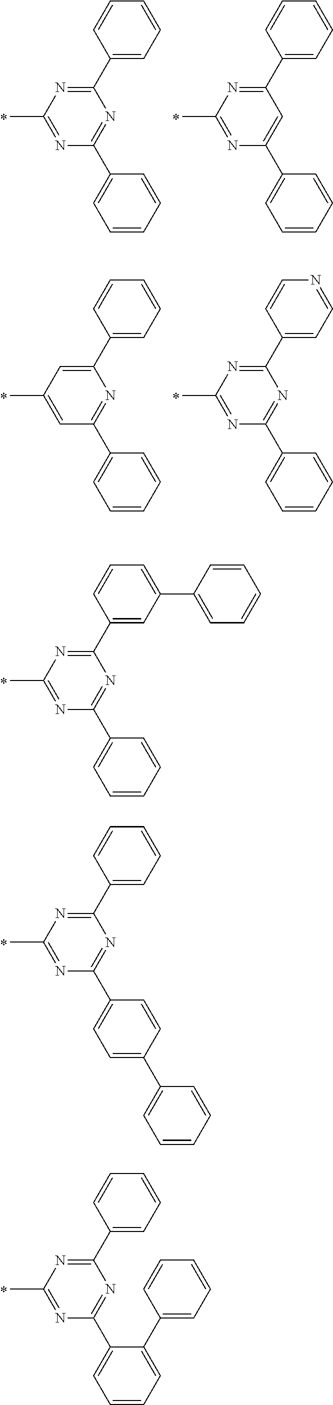

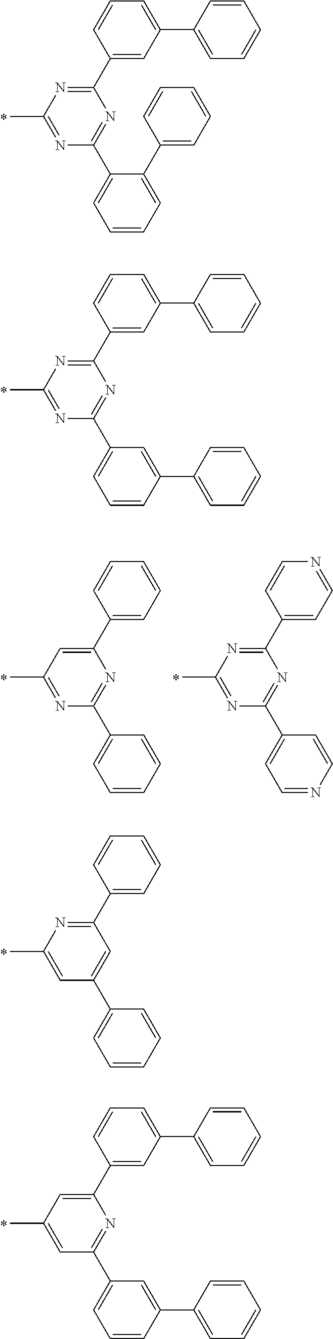

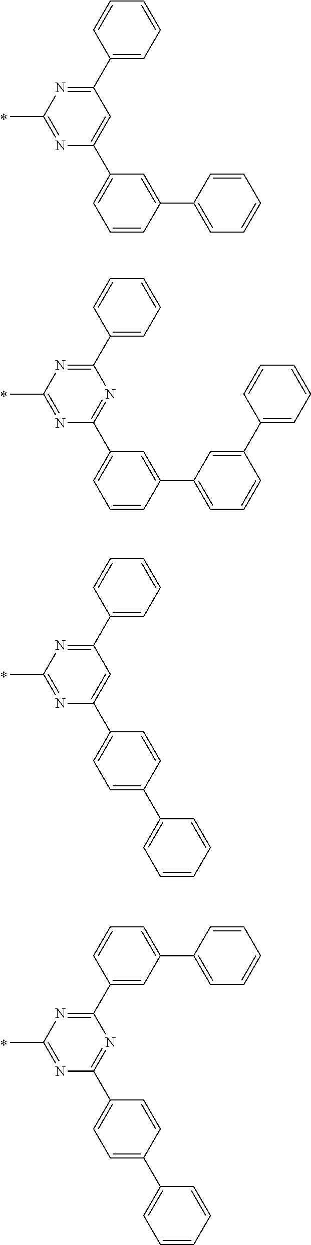

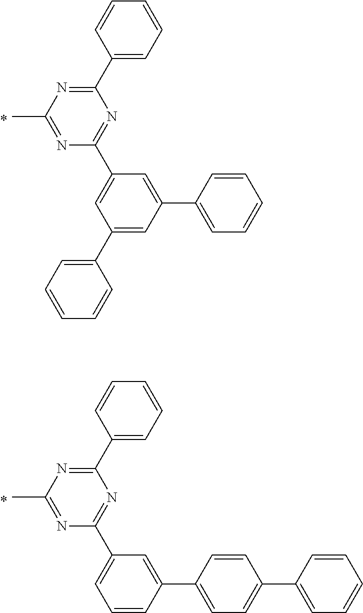 [SMILES: Cc1nc(-c2ccccc2)nc(-c2cc(-c3ccccc3)cc(-c3ccccc3)c2)n1 ; Cc1nc(-c2ccccc2)nc(-c2cccc(-c3ccc(-c4ccccc4)cc3)c2)n1]

In Groups dII and III, * is a linking point.

In an example embodiment of the present invention, L³ may be a single bond, a substituted or unsubstituted phenylene group, a substituted or unsubstituted biphenylene group, or a substituted or unsubstituted terphenylene group, and

for example a single bond or one of linking groups of Group IV.

[Group IV]

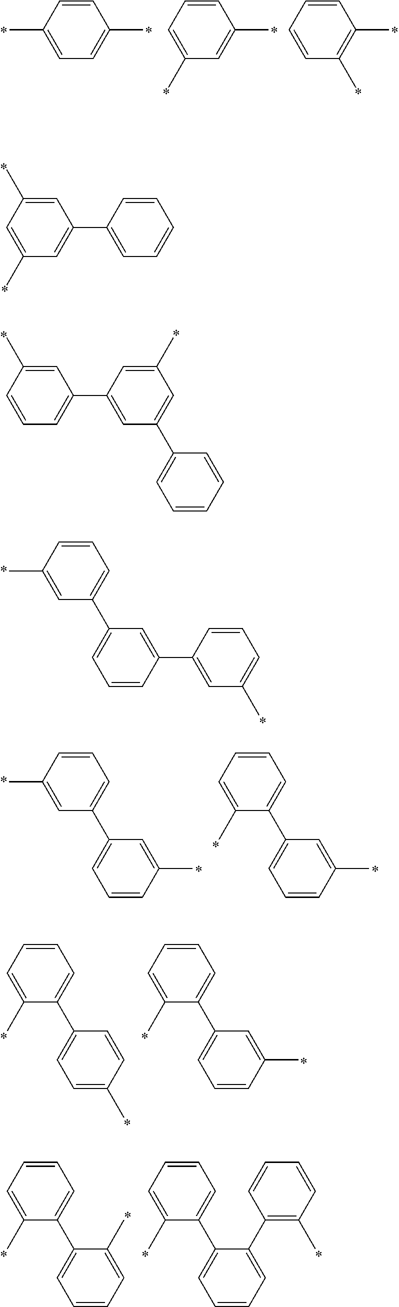

In Group IV, * is a linking point.

The second host compound may be, for example one of compounds of Group 2, but is not limited thereto.

[Group 2]

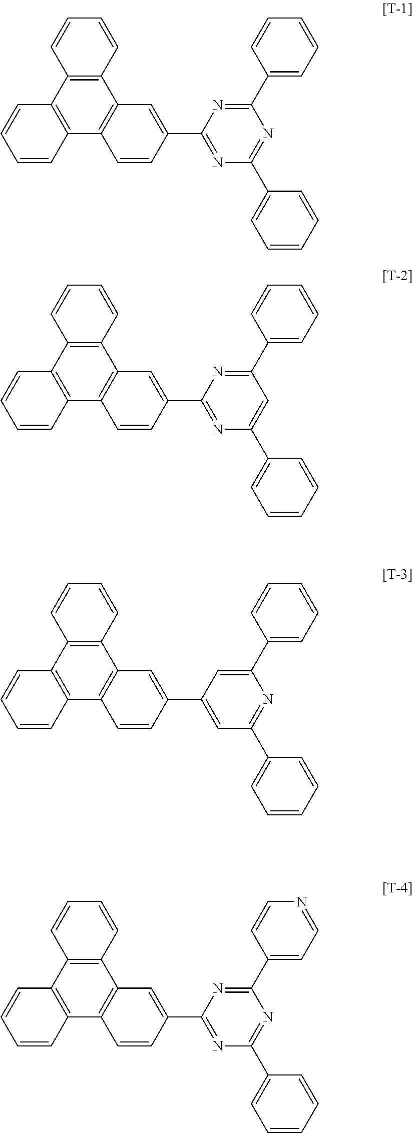

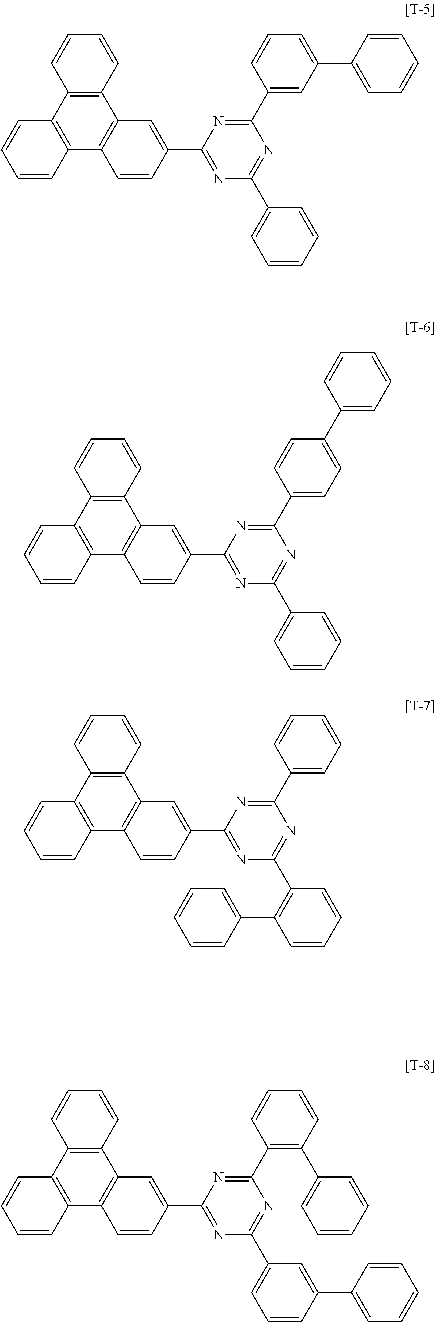

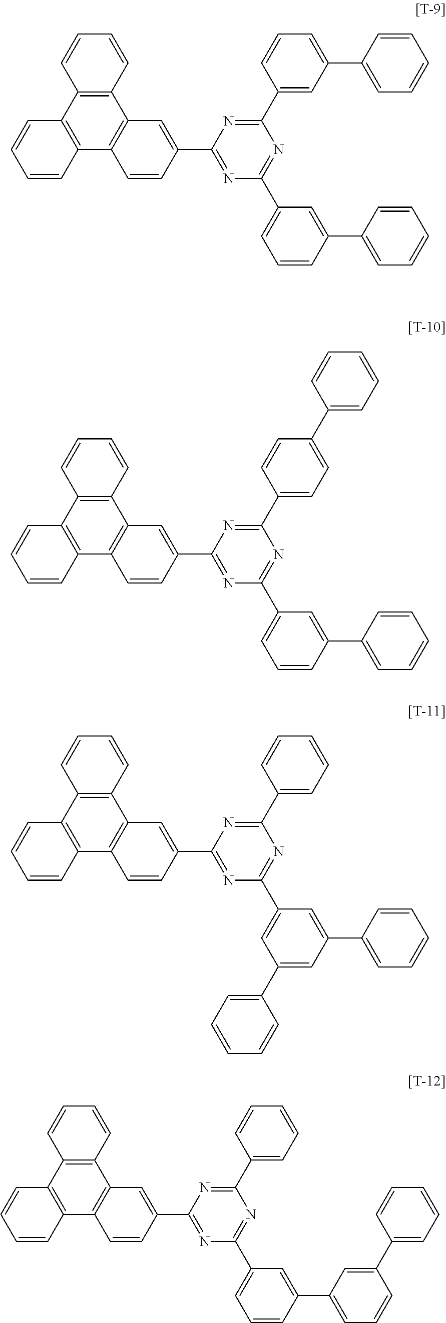 [SMILES: c1ccc(-c2cccc(-c3nc(-c4cccc(-c5ccccc5)c4)nc(-c4ccc5c6ccccc6c6ccccc6c5c4)n3)c2)cc1 ; c1ccc(-c2ccc(-c3nc(-c4cccc(-c5ccccc5)c4)nc(-c4ccc5c6ccccc6c6ccccc6c5c4)n3)cc2)cc1 ; c1ccc(-c2cc(-c3ccccc3)cc(-c3nc(-c4ccccc4)nc(-c4ccc5c6ccccc6c6ccccc6c5c4)n3)c2)cc1 ; c1ccc(-c2cccc(-c3cccc(-c4nc(-c5ccccc5)nc(-c5ccc6c7ccccc7c7ccccc7c6c5)n4)c3)c2)cc1]

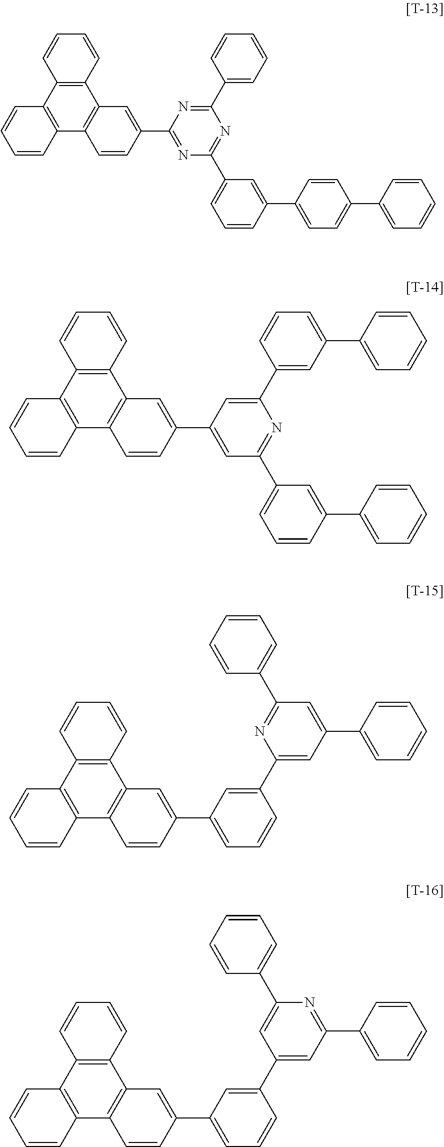 [SMILES: c1ccc(-c2ccc(-c3cccc(-c4nc(-c5ccccc5)nc(-c5ccc6c7ccccc7c7ccccc7c6c5)n4)c3)cc2)cc1 ; c1ccc(-c2cccc(-c3cc(-c4ccc5c6ccccc6c6ccccc6c5c4)cc(-c4cccc(-c5ccccc5)c4)n3)c2)cc1 ; c1ccc(-c2cc(-c3ccccc3)nc(-c3cccc(-c4ccc5c6ccccc6c6ccccc6c5c4)c3)c2)cc1 ; c1ccc(-c2cc(-c3cccc(-c4ccc5c6ccccc6c6ccccc6c5c4)c3)cc(-c3ccccc3)n2)cc1]

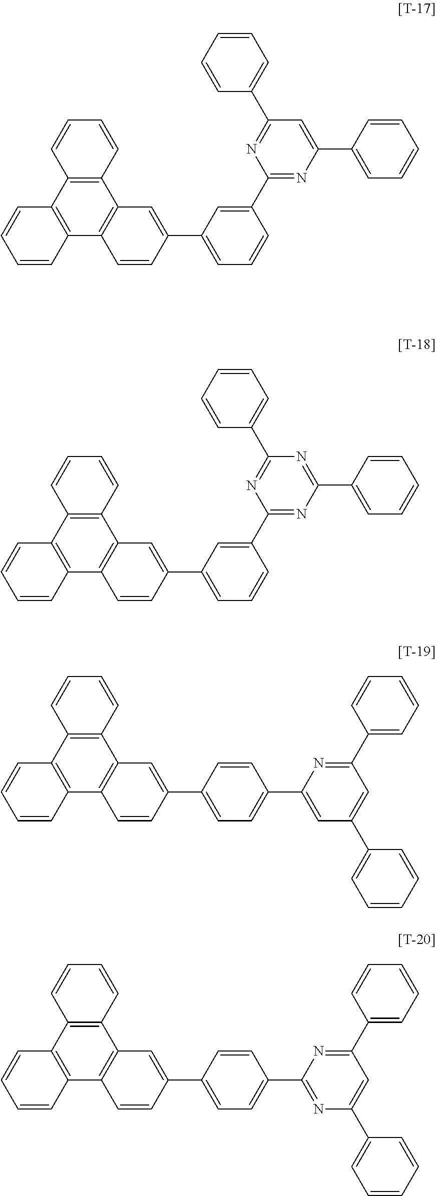 [SMILES: c1ccc(-c2cc(-c3ccccc3)nc(-c3cccc(-c4ccc5c6ccccc6c6ccccc6c5c4)c3)n2)cc1 ; c1ccc(-c2nc(-c3ccccc3)nc(-c3cccc(-c4ccc5c6ccccc6c6ccccc6c5c4)c3)n2)cc1 ; c1ccc(-c2cc(-c3ccccc3)nc(-c3ccc(-c4ccc5c6ccccc6c6ccccc6c5c4)cc3)c2)cc1 ; c1ccc(-c2cc(-c3ccccc3)nc(-c3ccc(-c4ccc5c6ccccc6c6ccccc6c5c4)cc3)n2)cc1]

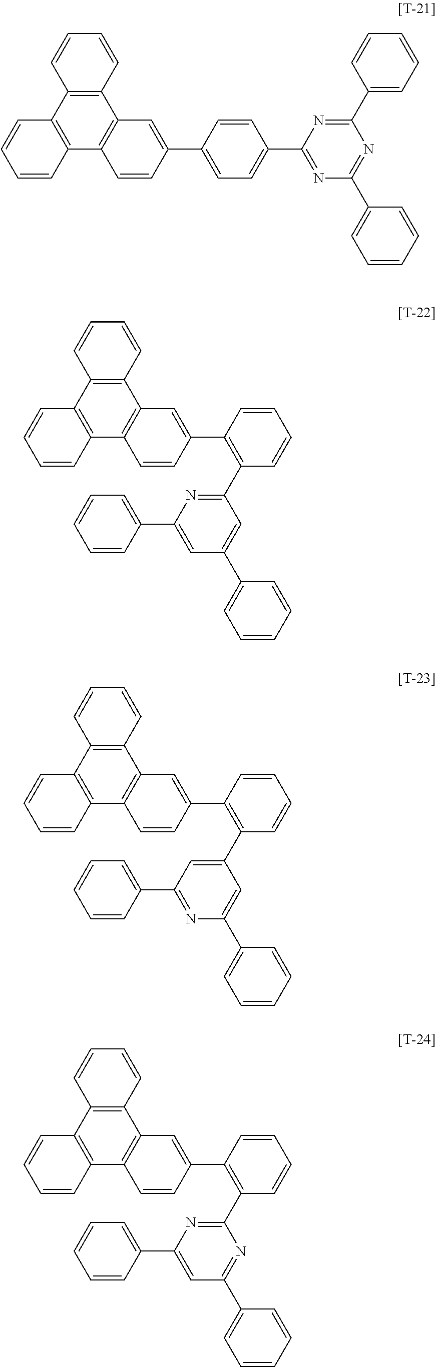 [SMILES: c1ccc(-c2nc(-c3ccccc3)nc(-c3ccc(-c4ccc5c6ccccc6c6ccccc6c5c4)cc3)n2)cc1 ; c1ccc(-c2cc(-c3ccccc3)nc(-c3ccccc3-c3ccc4c5ccccc5c5ccccc5c4c3)c2)cc1 ; c1ccc(-c2cc(-c3ccccc3-c3ccc4c5ccccc5c5ccccc5c4c3)cc(-c3ccccc3)n2)cc1 ; c1ccc(-c2cc(-c3ccccc3)nc(-c3ccccc3-c3ccc4c5ccccc5c5ccccc5c4c3)n2)cc1]

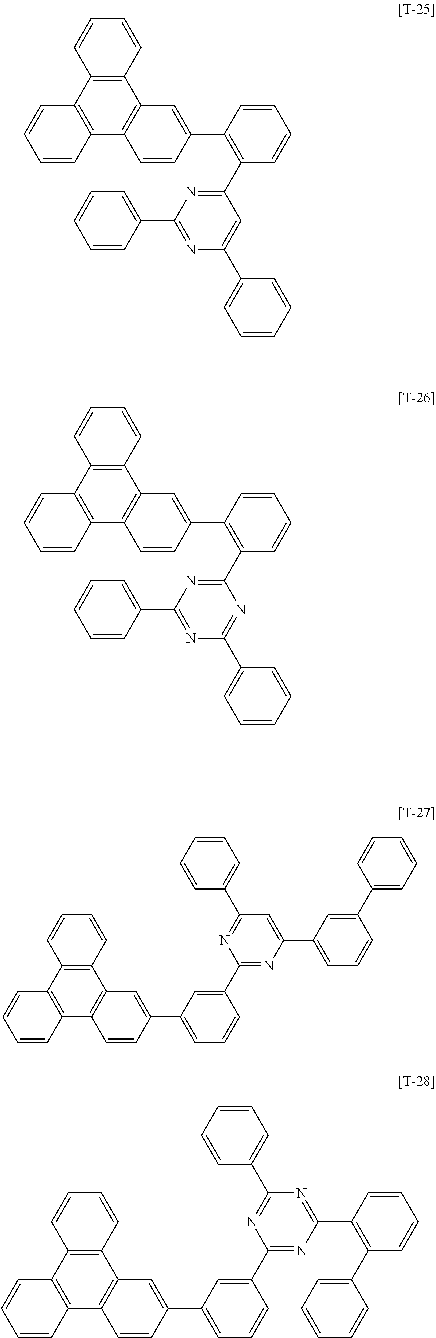 [SMILES: c1ccc(-c2cc(-c3ccccc3-c3ccc4c5ccccc5c5ccccc5c4c3)nc(-c3ccccc3)n2)cc1 ; c1ccc(-c2nc(-c3ccccc3)nc(-c3ccccc3-c3ccc4c5ccccc5c5ccccc5c4c3)n2)cc1 ; c1ccc(-c2cccc(-c3cc(-c4ccccc4)nc(-c4cccc(-c5ccc6c7ccccc7c7ccccc7c6c5)c4)n3)c2)cc1 ; c1ccc(-c2nc(-c3cccc(-c4ccc5c6ccccc6c6ccccc6c5c4)c3)nc(-c3ccccc3-c3ccccc3)n2)cc1]

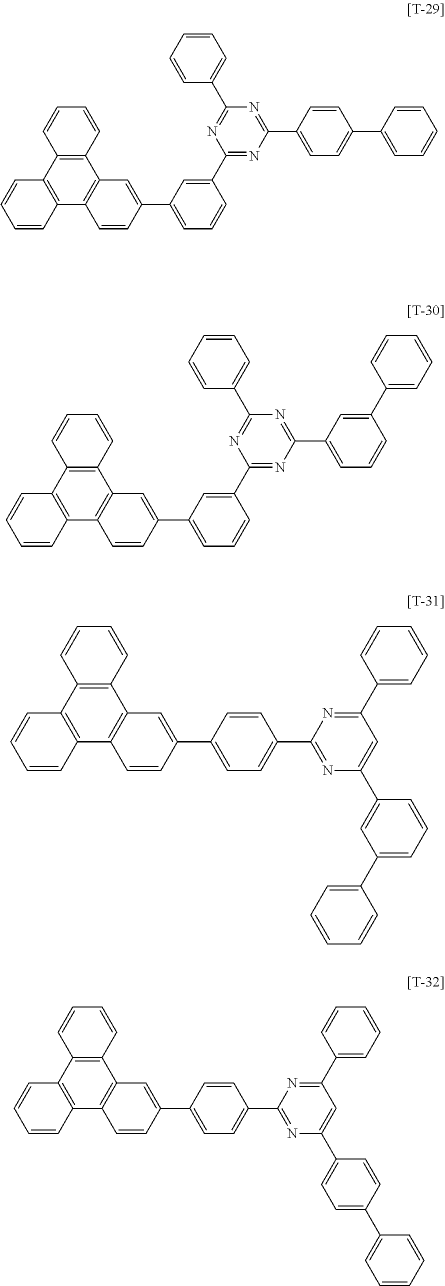 [SMILES: c1ccc(-c2ccc(-c3nc(-c4ccccc4)nc(-c4cccc(-c5ccc6c7ccccc7c7ccccc7c6c5)c4)n3)cc2)cc1 ; c1ccc(-c2cccc(-c3nc(-c4ccccc4)nc(-c4cccc(-c5ccc6c7ccccc7c7ccccc7c6c5)c4)n3)c2)cc1 ; c1ccc(-c2cccc(-c3cc(-c4ccccc4)nc(-c4ccc(-c5ccc6c7ccccc7c7ccccc7c6c5)cc4)n3)c2)cc1 ; c1ccc(-c2ccc(-c3cc(-c4ccccc4)nc(-c4ccc(-c5ccc6c7ccccc7c7ccccc7c6c5)cc4)n3)cc2)cc1]

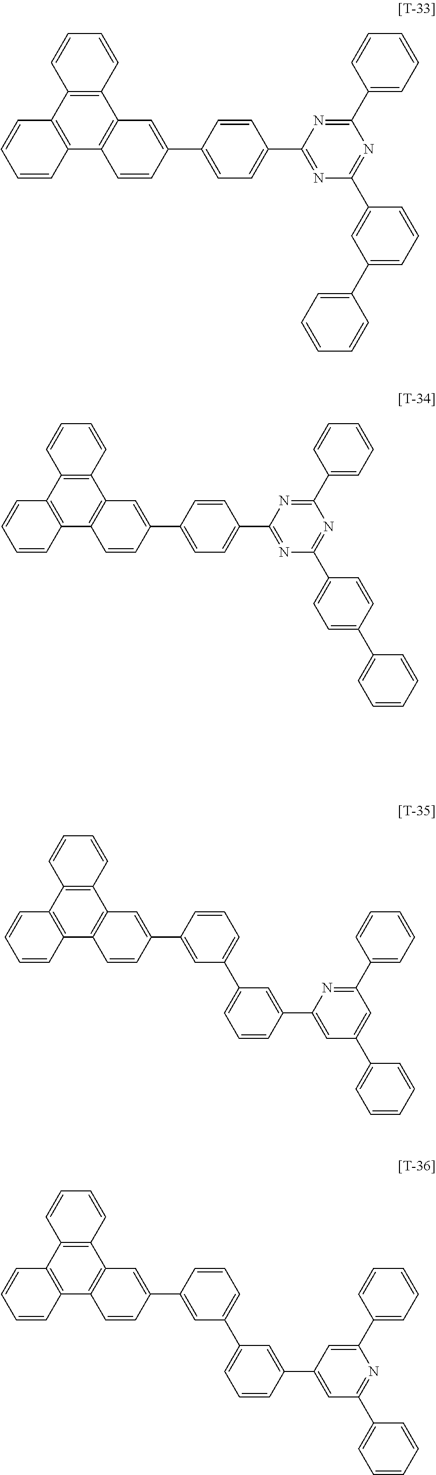 [SMILES: c1ccc(-c2cccc(-c3nc(-c4ccccc4)nc(-c4ccc(-c5ccc6c7ccccc7c7ccccc7c6c5)cc4)n3)c2)cc1 ; c1ccc(-c2ccc(-c3nc(-c4ccccc4)nc(-c4ccc(-c5ccc6c7ccccc7c7ccccc7c6c5)cc4)n3)cc2)cc1 ; c1ccc(-c2cc(-c3ccccc3)nc(-c3cccc(-c4cccc(-c5ccc6c7ccccc7c7ccccc7c6c5)c4)c3)c2)cc1 ; c1ccc(-c2cc(-c3cccc(-c4cccc(-c5ccc6c7ccccc7c7ccccc7c6c5)c4)c3)cc(-c3ccccc3)n2)cc1]

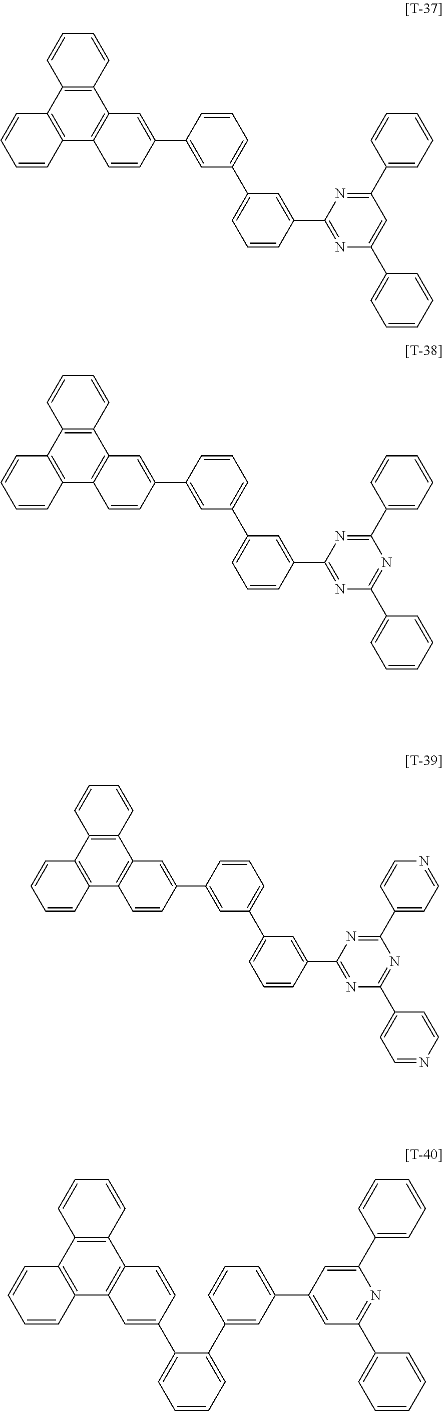 [SMILES: c1ccc(-c2cc(-c3ccccc3)nc(-c3cccc(-c4cccc(-c5ccc6c7ccccc7c7ccccc7c6c5)c4)c3)n2)cc1 ; c1ccc(-c2nc(-c3ccccc3)nc(-c3cccc(-c4cccc(-c5ccc6c7ccccc7c7ccccc7c6c5)c4)c3)n2)cc1 ; c1cc(-c2cccc(-c3nc(-c4ccncc4)nc(-c4ccncc4)n3)c2)cc(-c2ccc3c4ccccc4c4ccccc4c3c2)c1 ; c1ccc(-c2cc(-c3cccc(-c4ccccc4-c4ccc5c6ccccc6c6ccccc6c5c4)c3)cc(-c3ccccc3)n2)cc1]

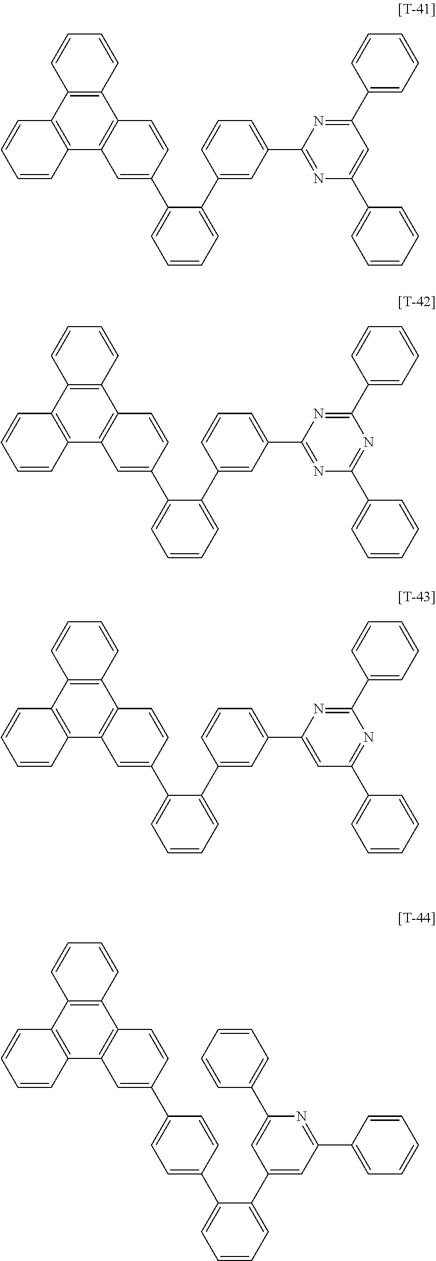 [SMILES: c1ccc(-c2cc(-c3ccccc3)nc(-c3cccc(-c4ccccc4-c4ccc5c6ccccc6c6ccccc6c5c4)c3)n2)cc1 ; c1ccc(-c2nc(-c3ccccc3)nc(-c3cccc(-c4ccccc4-c4ccc5c6ccccc6c6ccccc6c5c4)c3)n2)cc1 ; c1ccc(-c2cc(-c3cccc(-c4ccccc4-c4ccc5c6ccccc6c6ccccc6c5c4)c3)nc(-c3ccccc3)n2)cc1 ; c1ccc(-c2cc(-c3ccccc3-c3ccc(-c4ccc5c6ccccc6c6ccccc6c5c4)cc3)cc(-c3ccccc3)n2)cc1]

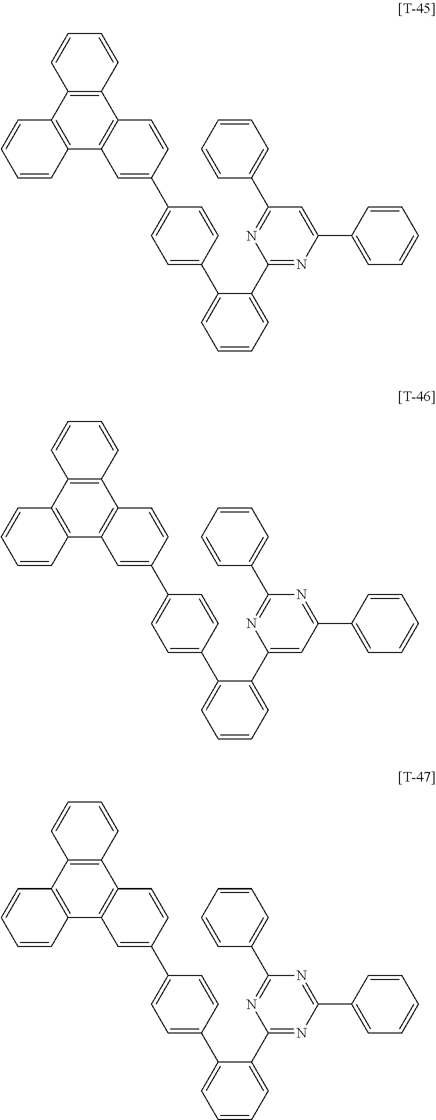 [SMILES: c1ccc(-c2cc(-c3ccccc3)nc(-c3ccccc3-c3ccc(-c4ccc5c6ccccc6c6ccccc6c5c4)cc3)n2)cc1 ; c1ccc(-c2cc(-c3ccccc3-c3ccc(-c4ccc5c6ccccc6c6ccccc6c5c4)cc3)nc(-c3ccccc3)n2)cc1 ; c1ccc(-c2nc(-c3ccccc3)nc(-c3ccccc3-c3ccc(-c4ccc5c6ccccc6c6ccccc6c5c4)cc3)n2)cc1]

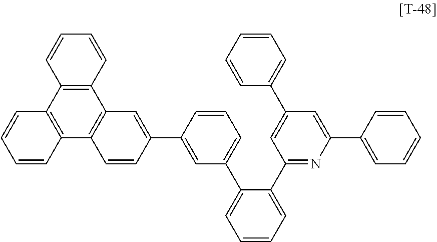 [SMILES: c1ccc(-c2cc(-c3ccccc3)nc(-c3ccccc3-c3cccc(-c4ccc5c6ccccc6c6ccccc6c5c4)c3)c2)cc1]

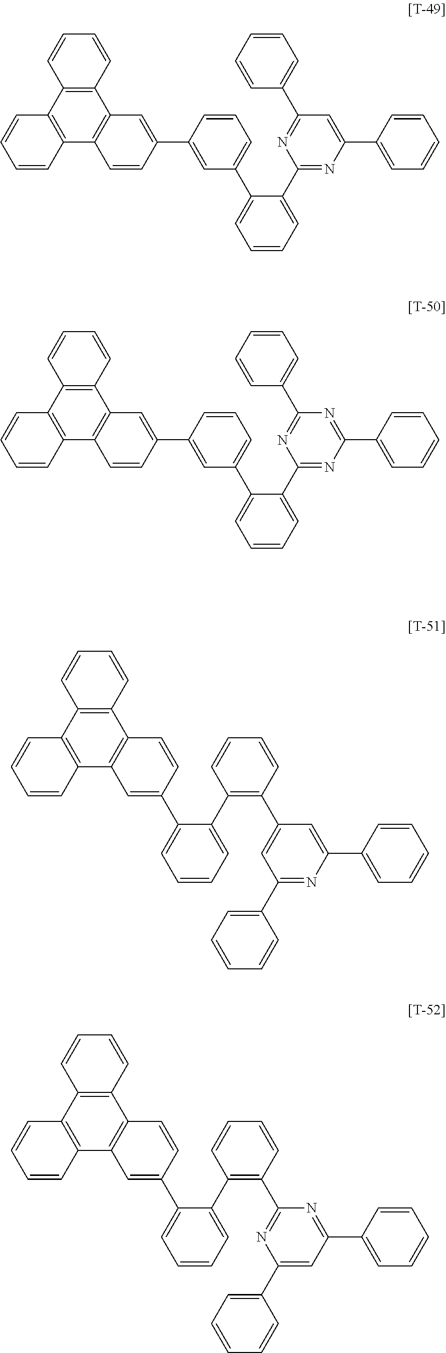 [SMILES: c1ccc(-c2cc(-c3ccccc3)nc(-c3ccccc3-c3cccc(-c4ccc5c6ccccc6c6ccccc6c5c4)c3)n2)cc1 ; c1ccc(-c2nc(-c3ccccc3)nc(-c3ccccc3-c3cccc(-c4ccc5c6ccccc6c6ccccc6c5c4)c3)n2)cc1 ; c1ccc(-c2cc(-c3ccccc3-c3ccccc3-c3ccc4c5ccccc5c5ccccc5c4c3)cc(-c3ccccc3)n2)cc1 ; c1ccc(-c2cc(-c3ccccc3)nc(-c3ccccc3-c3ccccc3-c3ccc4c5ccccc5c5ccccc5c4c3)n2)cc1]

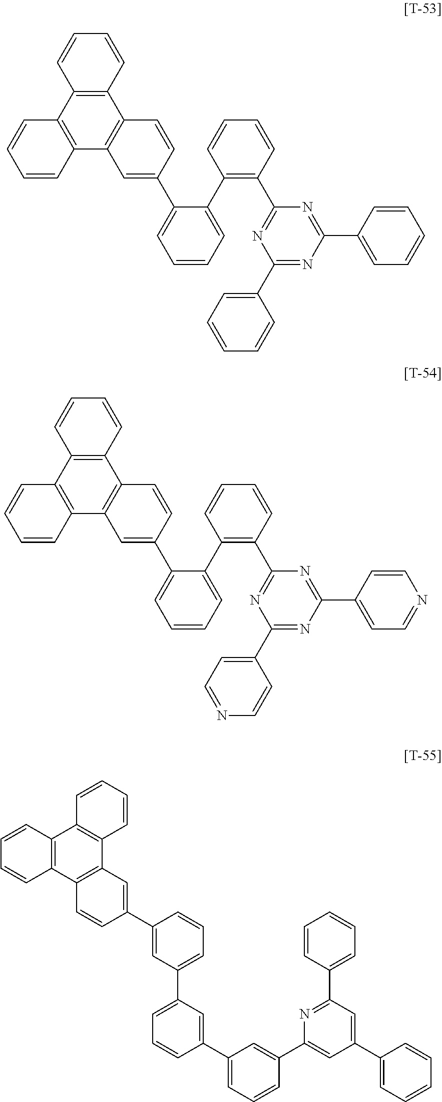 [SMILES: c1ccc(-c2nc(-c3ccccc3)nc(-c3ccccc3-c3ccccc3-c3ccc4c5ccccc5c5ccccc5c4c3)n2)cc1 ; c1ccc(-c2ccccc2-c2nc(-c3ccncc3)nc(-c3ccncc3)n2)c(-c2ccc3c4ccccc4c4ccccc4c3c2)c1 ; c1ccc(-c2cc(-c3ccccc3)nc(-c3cccc(-c4cccc(-c5cccc(-c6ccc7c8ccccc8c8ccccc8c7c6)c5)c4)c3)c2)cc1]

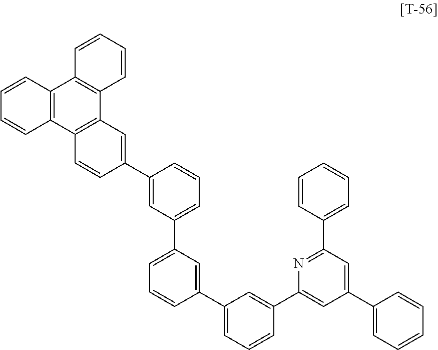 [SMILES: c1ccc(-c2cc(-c3ccccc3)nc(-c3cccc(-c4cccc(-c5cccc(-c6ccc7c8ccccc8c8ccccc8c7c6)c5)c4)c3)c2)cc1]

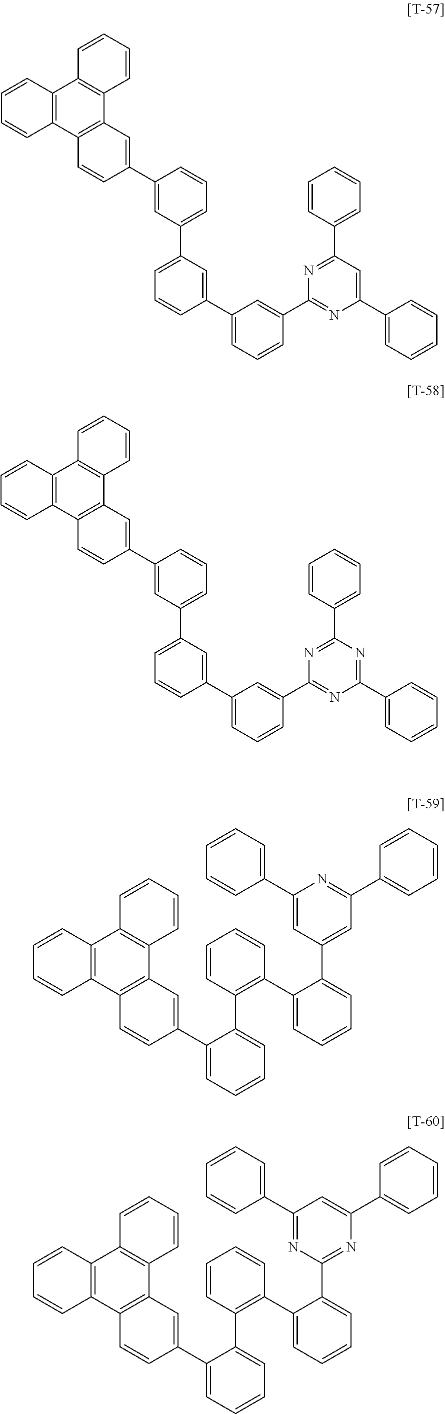 [SMILES: c1ccc(-c2cc(-c3ccccc3)nc(-c3cccc(-c4cccc(-c5cccc(-c6ccc7c8ccccc8c8ccccc8c7c6)c5)c4)c3)n2)cc1 ; c1ccc(-c2nc(-c3ccccc3)nc(-c3cccc(-c4cccc(-c5cccc(-c6ccc7c8ccccc8c8ccccc8c7c6)c5)c4)c3)n2)cc1 ; c1ccc(-c2cc(-c3ccccc3-c3ccccc3-c3ccccc3-c3ccc4c5ccccc5c5ccccc5c4c3)cc(-c3ccccc3)n2)cc1 ; c1ccc(-c2cc(-c3ccccc3)nc(-c3ccccc3-c3ccccc3-c3ccccc3-c3ccc4c5ccccc5c5ccccc5c4c3)n2)cc1]

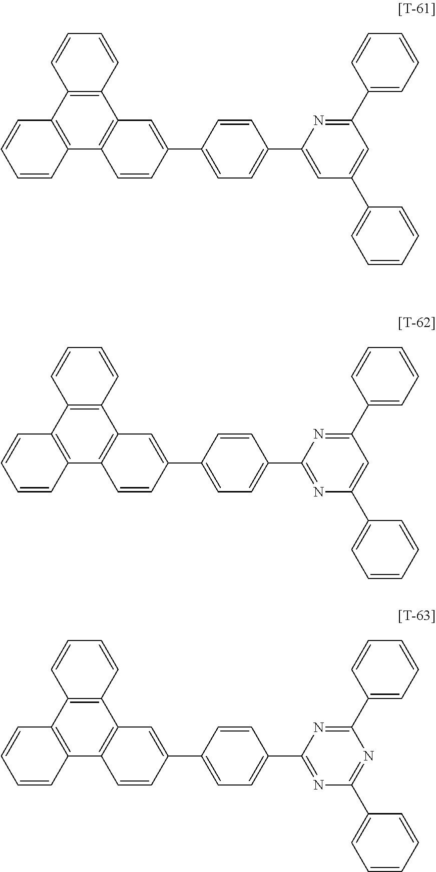 [SMILES: c1ccc(-c2cc(-c3ccccc3)nc(-c3ccc(-c4ccc5c6ccccc6c6ccccc6c5c4)cc3)c2)cc1 ; c1ccc(-c2cc(-c3ccccc3)nc(-c3ccc(-c4ccc5c6ccccc6c6ccccc6c5c4)cc3)n2)cc1 ; c1ccc(-c2nc(-c3ccccc3)nc(-c3ccc(-c4ccc5c6ccccc6c6ccccc6c5c4)cc3)n2)cc1]

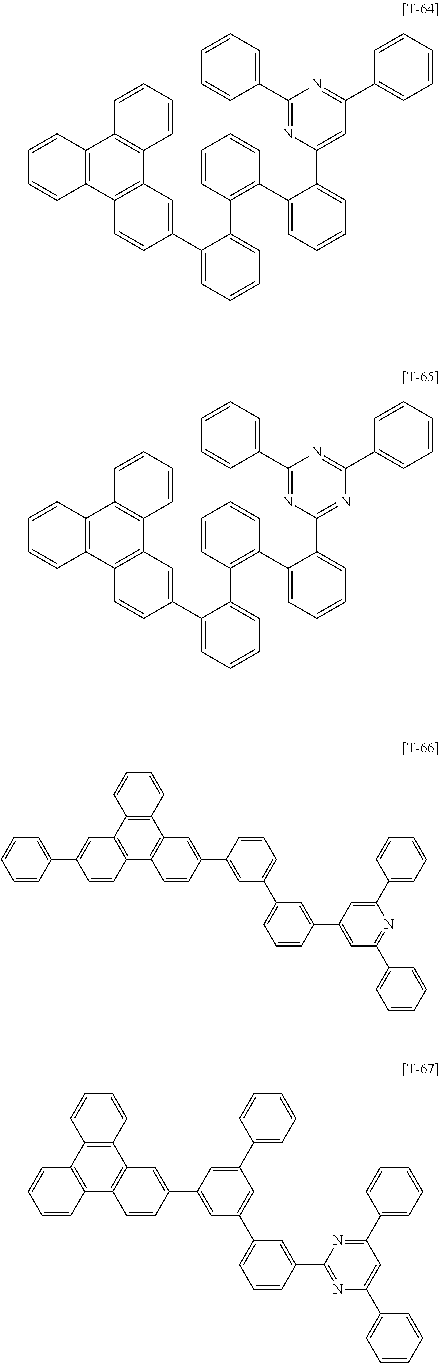 [SMILES: c1ccc(-c2cc(-c3ccccc3-c3ccccc3-c3ccccc3-c3ccc4c5ccccc5c5ccccc5c4c3)nc(-c3ccccc3)n2)cc1 ; c1ccc(-c2nc(-c3ccccc3)nc(-c3ccccc3-c3ccccc3-c3ccccc3-c3ccc4c5ccccc5c5ccccc5c4c3)n2)cc1 ; c1ccc(-c2ccc3c4ccc(-c5cccc(-c6cccc(-c7cc(-c8ccccc8)nc(-c8ccccc8)c7)c6)c5)cc4c4ccccc4c3c2)cc1 ; c1ccc(-c2cc(-c3cccc(-c4nc(-c5ccccc5)cc(-c5ccccc5)n4)c3)cc(-c3ccc4c5ccccc5c5ccccc5c4c3)c2)cc1]

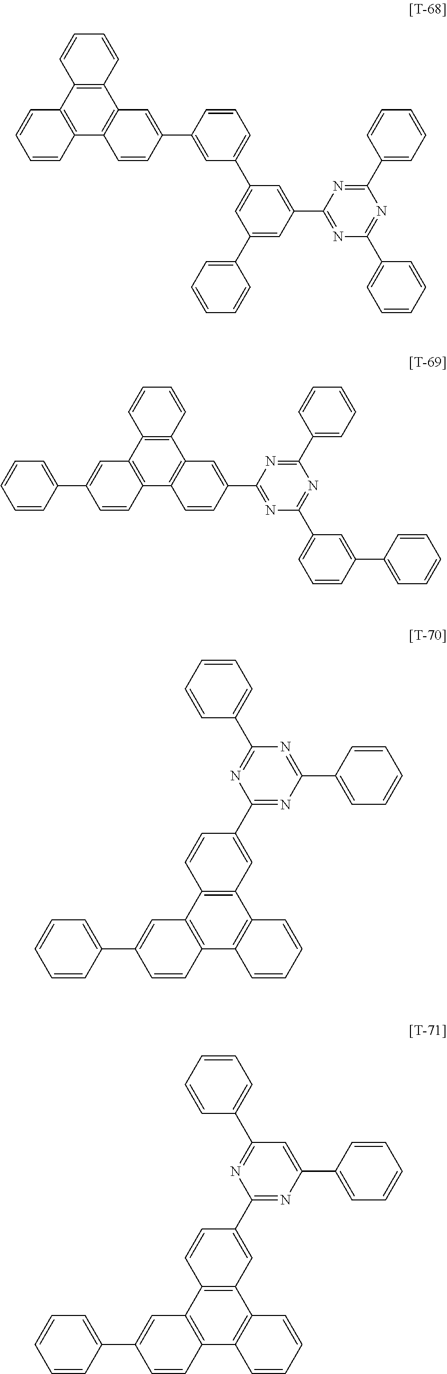 [SMILES: c1ccc(-c2cc(-c3cccc(-c4ccc5c6ccccc6c6ccccc6c5c4)c3)cc(-c3nc(-c4ccccc4)nc(-c4ccccc4)n3)c2)cc1 ; c1ccc(-c2cccc(-c3nc(-c4ccccc4)nc(-c4ccc5c6ccc(-c7ccccc7)cc6c6ccccc6c5c4)n3)c2)cc1 ; c1ccc(-c2ccc3c4ccccc4c4cc(-c5nc(-c6ccccc6)nc(-c6ccccc6)n5)ccc4c3c2)cc1 ; c1ccc(-c2ccc3c4ccccc4c4cc(-c5nc(-c6ccccc6)cc(-c6ccccc6)n5)ccc4c3c2)cc1]

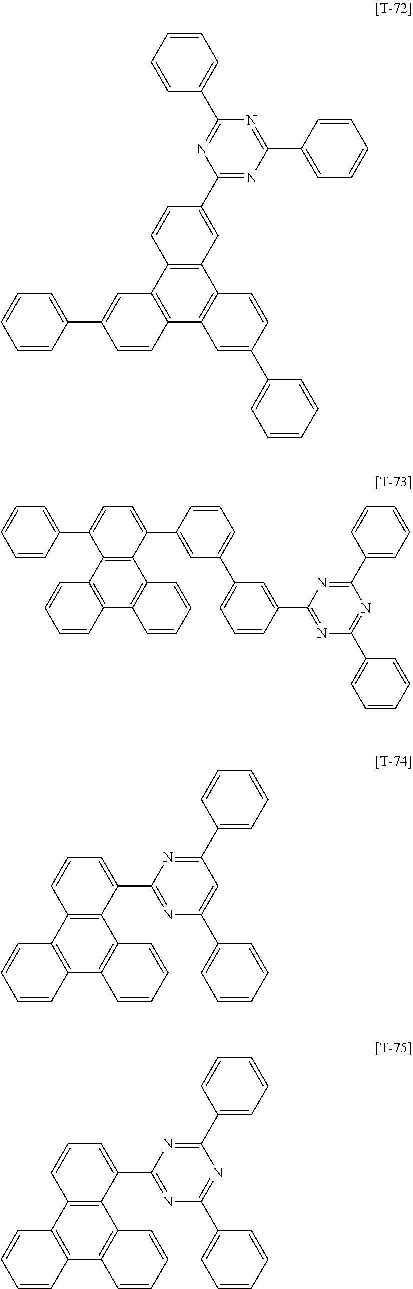 [SMILES: c1ccc(-c2ccc3c(c2)c2ccc(-c4ccccc4)cc2c2ccc(-c4nc(-c5ccccc5)nc(-c5ccccc5)n4)cc32)cc1 ; c1ccc(-c2nc(-c3ccccc3)nc(-c3cccc(-c4cccc(-c5ccc(-c6ccccc6)c6c7ccccc7c7ccccc7c56)c4)c3)n2)cc1 ; c1ccc(-c2cc(-c3ccccc3)nc(-c3cccc4c5ccccc5c5ccccc5c34)n2)cc1 ; c1ccc(-c2nc(-c3ccccc3)nc(-c3cccc4c5ccccc5c5ccccc5c34)n2)cc1]

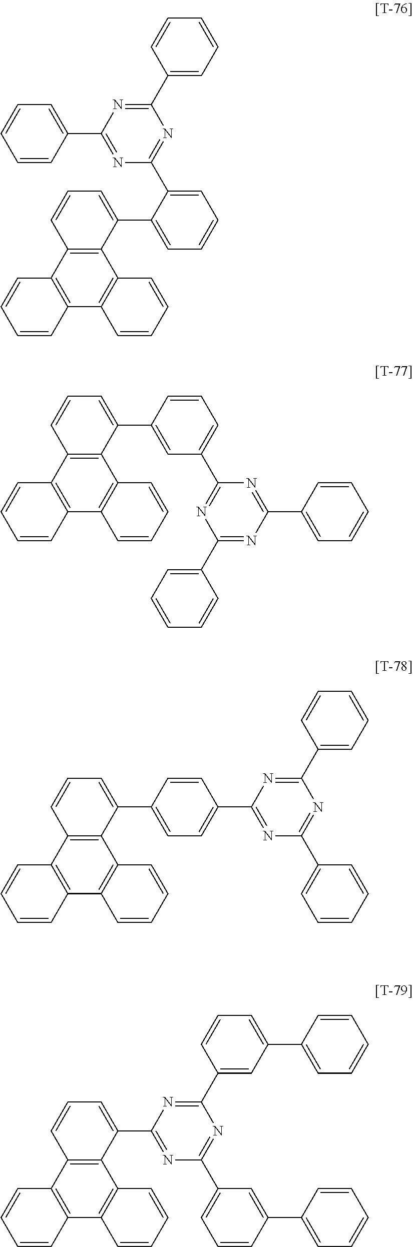 [SMILES: c1ccc(-c2nc(-c3ccccc3)nc(-c3ccccc3-c3cccc4c5ccccc5c5ccccc5c34)n2)cc1 ; c1ccc(-c2nc(-c3ccccc3)nc(-c3cccc(-c4cccc5c6ccccc6c6ccccc6c45)c3)n2)cc1 ; c1ccc(-c2nc(-c3ccccc3)nc(-c3ccc(-c4cccc5c6ccccc6c6ccccc6c45)cc3)n2)cc1 ; c1ccc(-c2cccc(-c3nc(-c4cccc(-c5ccccc5)c4)nc(-c4cccc5c6ccccc6c6ccccc6c45)n3)c2)cc1]

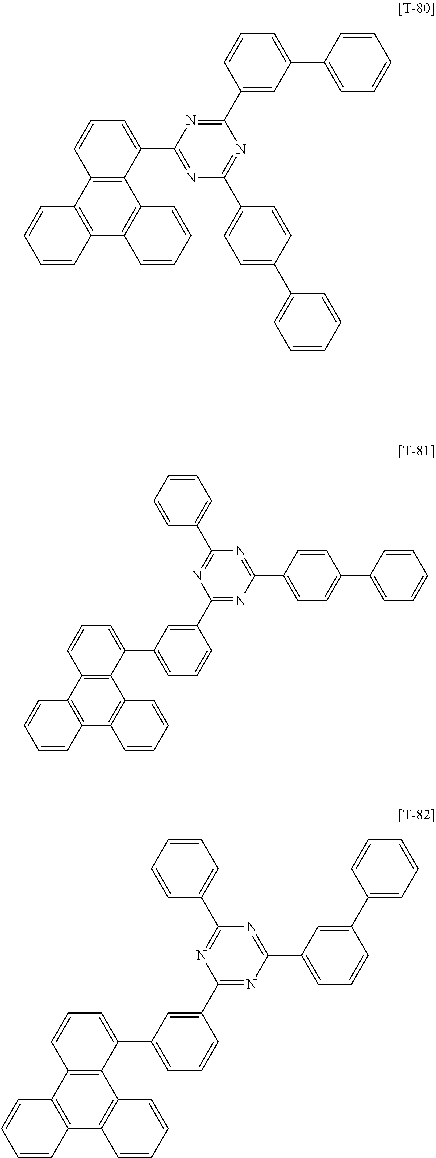 [SMILES: c1ccc(-c2ccc(-c3nc(-c4cccc(-c5ccccc5)c4)nc(-c4cccc5c6ccccc6c6ccccc6c45)n3)cc2)cc1 ; c1ccc(-c2ccc(-c3nc(-c4ccccc4)nc(-c4cccc(-c5cccc6c7ccccc7c7ccccc7c56)c4)n3)cc2)cc1 ; c1ccc(-c2cccc(-c3nc(-c4ccccc4)nc(-c4cccc(-c5cccc6c7ccccc7c7ccccc7c56)c4)n3)c2)cc1]

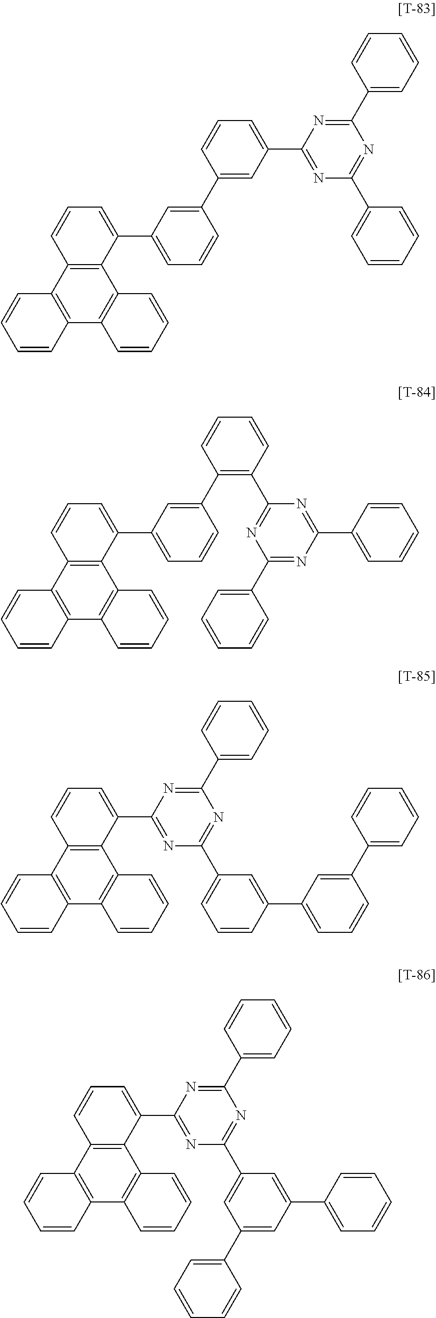 [SMILES: c1ccc(-c2nc(-c3ccccc3)nc(-c3cccc(-c4cccc(-c5cccc6c7ccccc7c7ccccc7c56)c4)c3)n2)cc1 ; c1ccc(-c2nc(-c3ccccc3)nc(-c3ccccc3-c3cccc(-c4cccc5c6ccccc6c6ccccc6c45)c3)n2)cc1 ; c1ccc(-c2cccc(-c3cccc(-c4nc(-c5ccccc5)nc(-c5cccc6c7ccccc7c7ccccc7c56)n4)c3)c2)cc1 ; c1ccc(-c2cc(-c3ccccc3)cc(-c3nc(-c4ccccc4)nc(-c4cccc5c6ccccc6c6ccccc6c45)n3)c2)cc1]

The first host compound and the second host compound may variously be combined to provide various compositions.

For example, a composition according to an example embodiment of the present invention includes a compound represented by Chemical Formula 1-C1 or Chemical Formula 1-E1 as a first host and the compound represented by Chemical Formula 3-I as a second host.

As described above, the first host compound is a compound having a relatively strong hole transport characteristics and the second host compound is a compound having a relatively strong electron transport characteristics, and thus improve luminous efficiency due to increased mobility of electrons and holes when they are used together compared with the compounds alone.

When a material having biased electron or hole characteristics is used to form a light-emitting layer, excitons in a device including the light-emitting layer are relatively more generated due to recombination of carriers on the interface between a light-emitting layer and an electron transport layer (ETL) or a hole transport layer (HTL). As a result, the molecular excitons in the light-emitting layer interact with charges on the interface of the transport layers and thus, cause a roll-off of sharply deteriorating efficiency and also, sharply deteriorate light emitting life-span characteristics. In order to solve the problems, the first and second hosts are simultaneously included in the light-emitting layer to make a light emitting region not be biased to either of the electron transport layer or the hole transport layer and a device capable of adjusting carrier balance in the light-emitting layer may be provided and thereby roll-off may be improved and life-span characteristics may be remarkably improved.

The first host compound and the second host compound may be, for example included in a weight ratio of 1:10 to 10:1. Specifically, they may be included in a weight ratio of 2:8 to 8:2, 3:7 to 7:3, 4:6 to 6:4, or 5:5, for example 4:6, or 5:5. Within the ranges, bipolar characteristics may be effectively realized to improve efficiency and life-span simultaneously.

The composition may further include at least one compound in addition to the first host compound and the second host compound.

The composition may further include a dopant. The dopant may be a red, green, or blue dopant, for example a phosphorescent dopant.

The dopant is mixed with the first host compound and the second host compound in a small amount to cause light emission, and may be generally a material such as a metal complex that emits light by multiple excitation into a triplet or more. The dopant may be, for example an inorganic, organic, or organic/inorganic compound, and one or more kinds thereof may be used.

Examples of the phosphorescent dopant may be an organic metallic compound including Ir, Pt, Os, Ti, Zr, Hf, Eu, Tb, Tm, Fe, Co, Ni, Ru, Rh, Pd, or a combination thereof. The phosphorescent dopant may be, for example a compound represented by Chemical Formula Z, but is not limited thereto.

L₂MX [Chemical Formula Z]

In Chemical Formula Z, M is a metal, and L and X are the same or different, and are a ligand to form a complex compound with M.

The M may be, for example Ir, Pt, Os, Ti, Zr, Hf, Eu, Tb, Tm, Fe, Co, Ni, Ru, Rh, Pd, or a combination thereof and the L and X may be, for example a bidendate ligand.

The composition may be formed using a dry film formation method or a solution process.

Hereinafter, an organic optoelectric device according to another embodiment is described.

An organic optoelectric device according to another embodiment includes an anode and a cathode facing each other and at least one organic layer between the anode and the cathode, and the organic layer includes the composition for an organic optoelectric device.

Herein, an organic light emitting diode as one example of an organic optoelectric device is described referring to drawings.

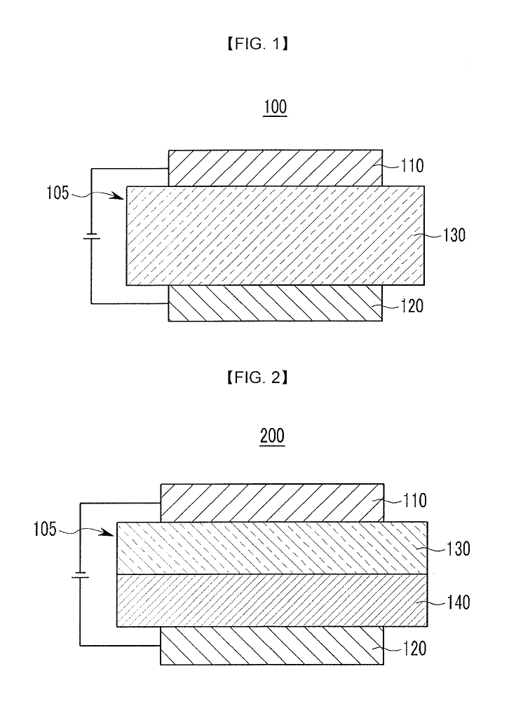

FIGS. 1 and 2 are cross-sectional views showing organic light emitting diodes according to each embodiment.

Referring to FIG. 1, an organic light emitting diodes 100 according to an embodiment includes an anode 120 and a cathode 110 and an organic layer 105 between the anode 120 and the cathode 110.

The anode 120 may be made of a conductor having a large work function to help hole injection, and may be for example metal, metal oxide and/or a conductive polymer. The anode 120 may be, for example a metal nickel, platinum, vanadium, chromium, copper, zinc, gold, and the like or an alloy thereof; metal oxide such as zinc oxide, indium oxide, indium tin oxide (ITO), indium zinc oxide (IZO), and the like; a combination of metal and oxide such as ZnO and Al or SnO₂ and Sb; a conductive polymer such as poly(3-methylthiophene), poly(3,4-(ethylene-1,2-dioxy)thiophene) (PEDT), polypyrrole, and polyaniline, but is not limited thereto.

The cathode 110 may be made of a conductor having a small work function to help electron injection, and may be for example metal, metal oxide and/or a conductive polymer. The cathode 110 may be for example a metal or an alloy thereof such as magnesium, calcium, sodium, potassium, titanium, indium, yttrium, lithium, gadolinium, aluminum silver, tin, lead, cesium, barium, and the like; a multi-layer structure material such as LiF/Al, LiO₂/Al, LiF/Ca, LiF/Al and BaF₂/Ca, but is not limited thereto.

The organic layer 105 includes an emission layer 130 including the composition.

The emission layer 130 may include, for example the composition.

Referring to FIG. 2, an organic light emitting diode 200 includes a hole auxiliary layer 140 in addition to the emission layer 130. The hole auxiliary layer 140 increases hole injection and/or hole mobility and blocks electrons between the anode 120 and the emission layer 130. The hole auxiliary layer 140 may be, for example a hole transport layer, a hole injection layer, and/or an electron blocking layer, and may include at least one layer.

In an embodiment of the present invention, in FIG. 1 or 2, an organic light emitting diode may further include an electron transport layer, an electron injection layer, a hole injection layer as the organic layer 105.

The organic light emitting diodes 100 and 200 may be manufactured by forming an anode or a cathode on a substrate, forming an organic layer using a dry film formation method such as a vacuum deposition method (evaporation), sputtering, plasma plating, and ion plating, and forming a cathode or an anode thereon.

The organic light emitting diode may be applied to an organic light emitting diode (OLED) display.

Hereinafter, the embodiments are illustrated in more detail with reference to examples. These examples, however, are not in any sense to be interpreted as limiting the scope of the invention.

(Preparation of Composition for Organic Optoelectric Device)

Hereinafter, a starting material and a reactant used in Examples and Synthesis Examples were purchased from Sigma-Aldrich Co. Ltd. or TCI Inc. as far as there in no particular comment and may be easily synthesized as a publicly known material.

In the following Synthesis Examples, when “B′ is used instead of ‘A’”, the amounts of ‘A’ and ‘B’ are the same as based on a mole equivalent.

As specific examples of the compound for an organic optoelectric device of the present invention, the compound of Chemical Formula 1 is synthesized by the following reaction schemes.

Synthesis of First Host Compound

### Synthesis Example 1: Synthesis of Compound C-1

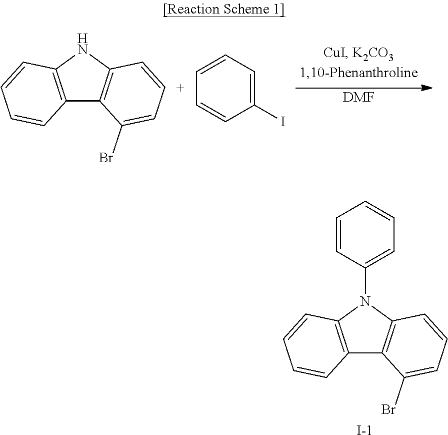

First Step: Synthesis of Intermediate I-1

4-bromo-9H-carbazole (50.4 g, 204.8 mmol) was dissolved in 500 mL of dimethylformamide (DMF) in an nitrogen environment, iodobenzene (62.7 g, 307.3 mmol) and copper iodide (7.8 g, 41 mmol), potassium carbonate (K₂CO₃) (42.5 g, 307.3 mmol), and 1,10-phenanthroline (7.4 g, 41 mmol) were added thereto, and the mixture was heated and refluxed at 140° C. for 12 hours. When the reaction was complete, water was added to the reaction solution to precipitate a solid, and then, DCM was used for an extraction after filtering the solid. The obtained residue was separated and purified through silica gel column chromatography to obtain Intermediate I-1 (60 g and 91%).

HRMS (70 eV, EI+): m/z calcd for C18H12BrN: 322.20, found 322.

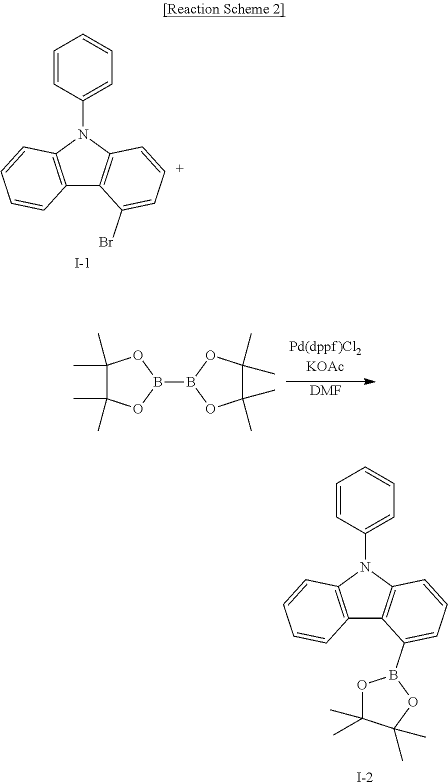

### Second Step: Synthesis of Intermediate I-2

The Intermediate I-1 (58.6 g, 181.8 mmol) and bis(pinacolato)diboron (60.0 g, 236.4 mmol) were dissolved in 700 mL of dimethylformamide (DMF) in an nitrogen environment, (1,1′-bis(diphenylphosphine)ferrocene)dichloropalladium (II) (Pd(dppf)) (7.4 g, 9.1 mmol) and potassium acetate (KOAc) (26.8 g, 272.8 mmol) were added thereto at 140° C., and the mixture was heated and refluxed for 12 hours. When the reaction was completed, water was added thereto to precipitate a solid, and then, DCM was twice used for an extraction after filtering the solid. This obtained residue was recrystallized and purified with a mixed solution of DCM: n-hexane to obtain Intermediate I-2 (47.0 g, 70%).

HRMS (70 eV, EI+): m/z calcd for C24H24BNO2: 369.26, found 369.

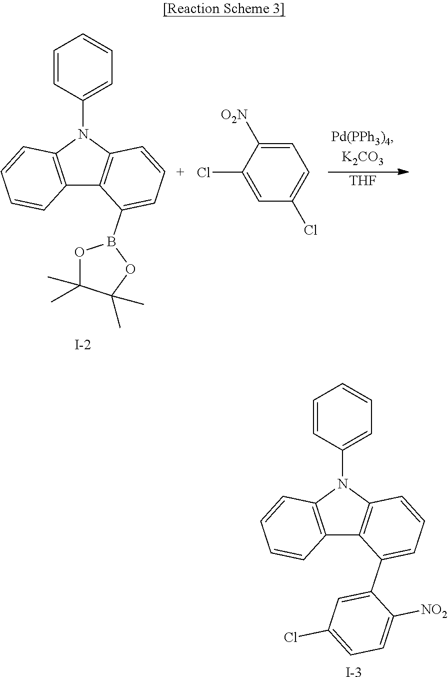

Third Step: Synthesis of Intermediate I-3

The Intermediate I-2 (36.4 g, 98.5 mmol) was dissolved in 1 L of tetrahydrofuran (THF) in an nitrogen environment, 2,4-dichloro-1-nitrobenzene (22.7 g, 118.2 mmol) and tetrakis(triphenylphosphine)palladium (Pd(PPh₃)₄) (5.7 g, 4.9 mmol) were added thereto, and the mixture was stirred. Potassium carbonate saturated in water (K₂CO₃, 27.3 g, 197.1 mmol) was added thereto, and the obtained mixture was heated and refluxed at 80° C. for 12 hours. After completing the reaction, water was added to the reaction solution, the mixture was extracted with dichloromethane (DCM) and treated with anhydrous MgSO₄ to remove moisture, and the resultant was filtered and concentrated under a reduced pressure

This obtained residue was separated and purified through flash column chromatography to obtain Intermediate I-3 (27.5 g, 70%).

HRMS (70 eV, EI+): m/z calcd for C24H15ClN2O2: 398.84, found 399.

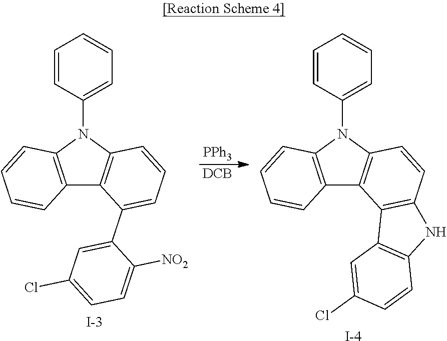

Fourth Step: Synthesis of Intermediate I-4

The intermediate I-3 (24.0 g, 60.0 mmol) was dissolved in 250 mL of dichlorobenzene (DCB) in an nitrogen environment, triphenylphosphine (78.7 g, 299.9 mmol) was added thereto, and the mixture was heated and refluxed at 180° C. for 12 hours. When the reaction was complete, water was added to the reaction solution, the mixture was extracted with dichloromethane (DCM) and treated with anhydrous MgSO₄ to remove moisture, and the resultant was filtered and concentrated under a reduced pressure. The obtained residue was separated and purified through flash column chromatography to obtain Intermediate I-4 (11 g and 50%).

HRMS (70 eV, EI+): m/z calcd for C24H15ClN2: 366.84, found 367.

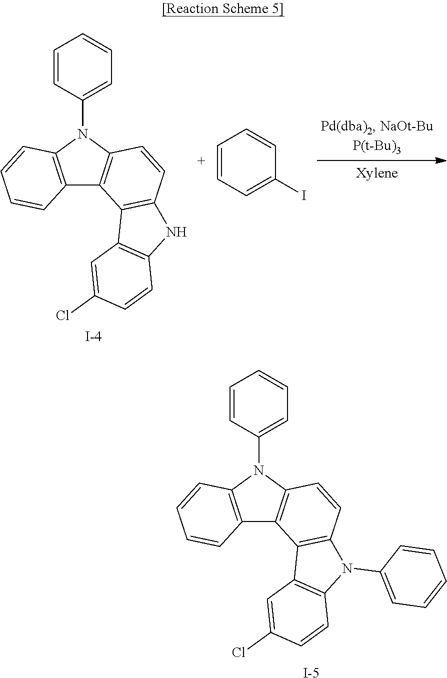

Fifth Step: Synthesis of Intermediate I-5

The Intermediate I-4 (11 g, 30.0 mmol) was dissolved in 150 mL of xylene in an nitrogen environment, iodobenzene (62.7 g, 307.3 mmol), Pd(dba)₂ (0.86 g, 1.5 mmol), sodium t-butoxide (5.8 g, 60.1 mmol), and tri-tert-butylphosphine (1.5 g, 3.0 mmol) were added thereto, and the mixture was heated and refluxed at 130° C. for 10 hours. When the reaction was complete, water was added to precipitate a solid, and DCM was used for an extraction after filtering the solid. The obtained residue was separated and purified through silica gel column chromatography to obtain Intermediate I-5 (11.5 g, 86%).

HRMS (70 eV, EI+): m/z calcd for C30H19ClN2: 442.94, found 443.

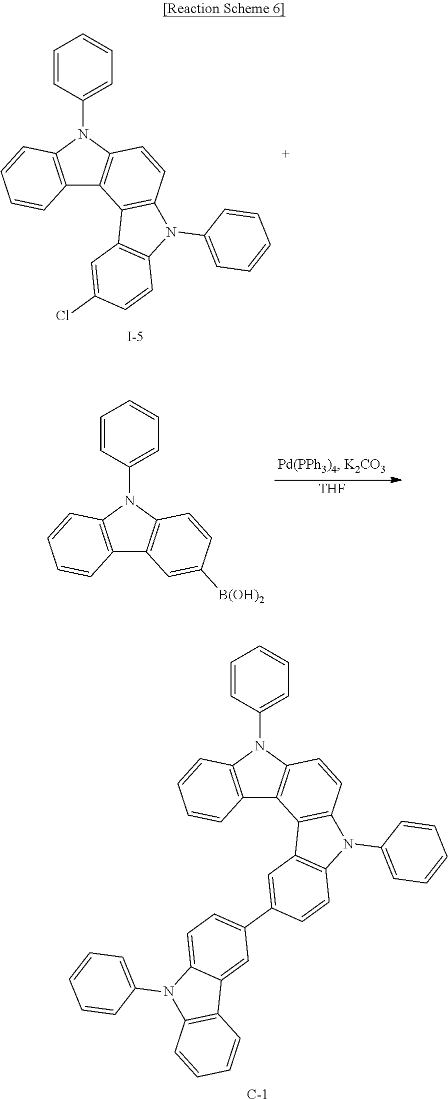

Sixth Step: Synthesis of Compound C-1

The Intermediate I-5 (5.8 g, 12.9 mmol) was dissolved in 150 mL of tetrahydrofuran (THF) in an nitrogen environment, 9-phenyl-9H-carbazol-3-yl boronic acid (4.5 g, 15.6 mmol) and tetrakis(triphenylphosphine)palladium (Pd(PPh₃)₄) (0.75 g, 0.65 mmol) were added thereto, and the mixture was stirred. Potassium carbonate saturated in water (K₂CO₃, 3.6 g, 26.0 mmol) was added thereto, and the obtained mixture was heated and refluxed at 80° C. for 12 hours. When the reaction was complete, water was added thereto, the mixture was extracted with dichloromethane (DCM) and treated with anhydrous MgSO₄ to remove moisture, and the resultant was filtered and concentrated under a reduced pressure. This obtained residue was separated and purified through flash column chromatography to obtain Compound C-1 (7.0 g, 83%).

HRMS (70 eV, EI+): m/z calcd for C₄₈H₃₁N₃: 649.78, found 649.

### Synthesis Example 2: Synthesis of Compound C-2

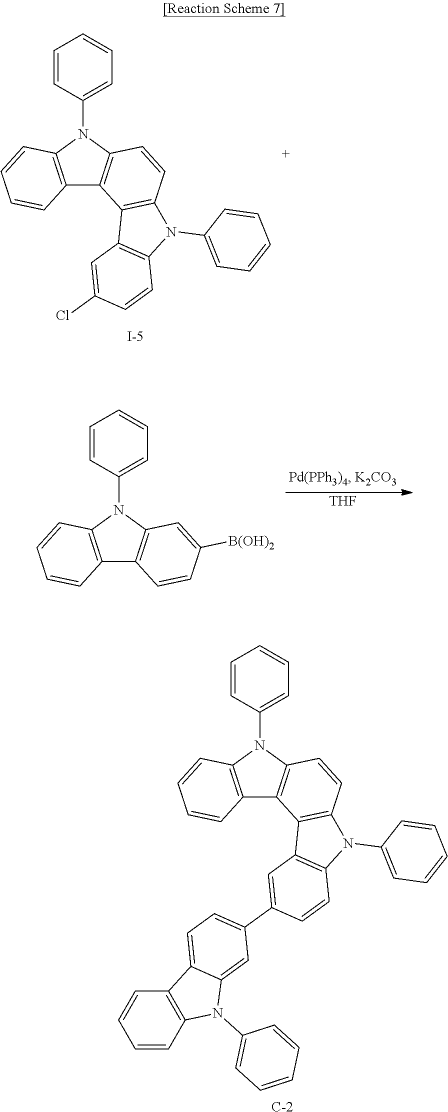

Compound C-2 (6.8 g, 79%) was obtained according to the same method as the sixth step of Synthesis Example 1 except for using 9-phenyl-9H-carbazol-2-yl boronic acid instead of the 9-phenyl-9H-carbazol-3-ylboronic acid.

HRMS (70 eV, EI+): m/z calcd for C₄₈H₃₁N₃: 649.78, found 649.

### Synthesis Example 3: Synthesis of Compound E-1

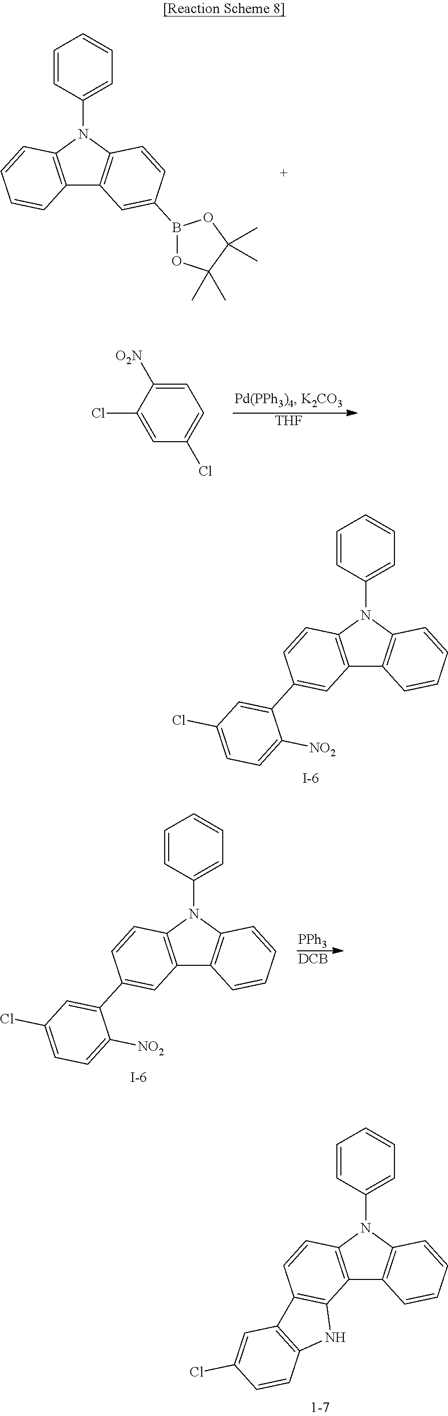

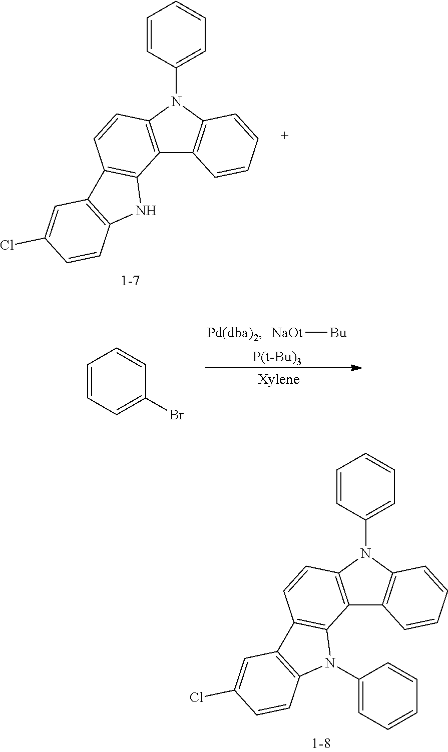

Second Step: Synthesis of Intermediate I-8

Intermediate I-8 (35.2 g, 94%) was obtained through the same reaction as the third to fifth steps of Synthesis Example 1 except for using 9-phenyl-3-(4,4,5,5-tetramethyl-1,3,2-dioxaborolan-2-yl)-9H-carbazole instead of the Intermediate I-2 in the third step.

HRMS (70 eV, EI+): m/z calcd for C30H19ClN2: 442.94, found 443.

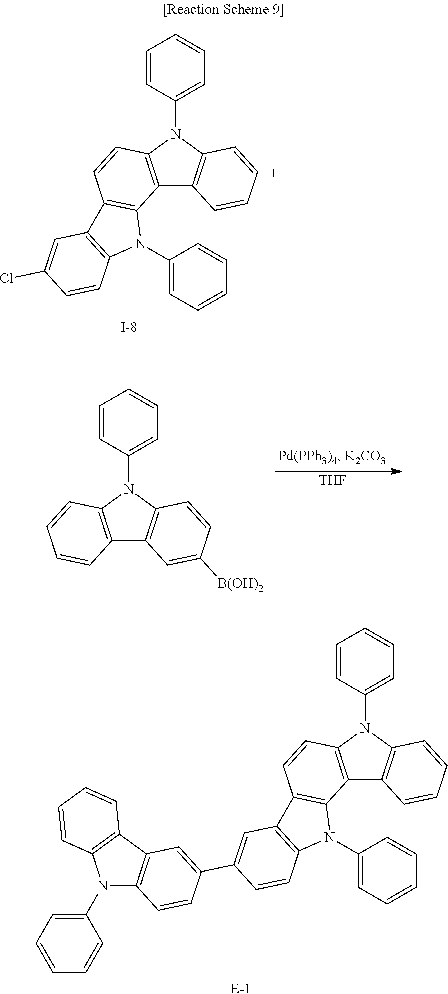

Second Step: Synthesis of Compound E-1

Compound E-1 (13.3 g, 79%) was obtained through the same reaction as the sixth step of Synthesis Example 1 except for using the intermediate I-8 (11.5 g, 25.9 mmol) instead of the Intermediate I-5.

HRMS (70 eV, EI+): m/z calcd for C₄₈H₃₁N₃: 649.78, found 649.

### Synthesis Example 4: Synthesis of Compound E-2

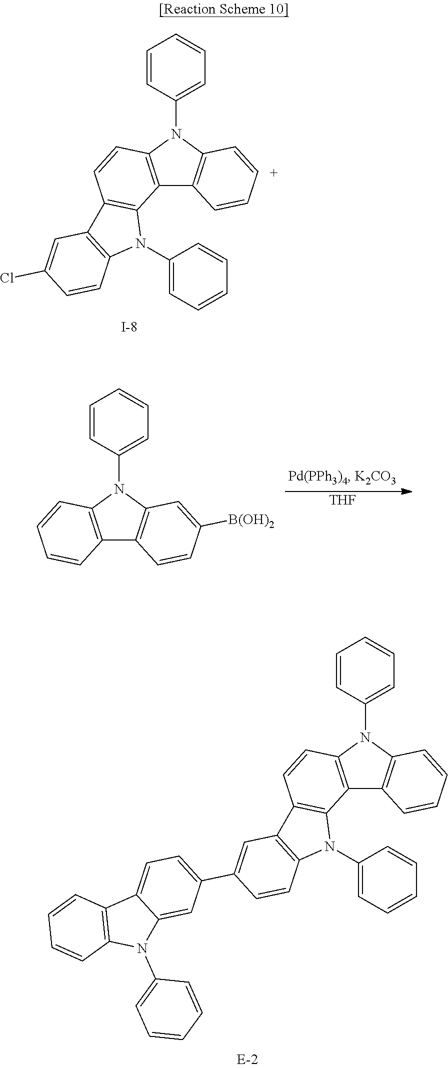

Compound E-2 (6.9 g, 80%) was obtained through the same reaction as Synthesis Example 2 except for using the Intermediate I-8 instead of the Intermediate I-5.

HRMS (70 eV, EI+): m/z calcd for C₄₈H₃₁N₃: 649.78, found 649.

Synthesis of Second Host Compound

### Synthesis Example 5: Synthesis of Compound T-9

Compound T-9 was synthesized according to the same synthesis method as Compound 5 among the Synthesis Example methods described in Patent Laid Open US 2015-0349268.

### Synthesis Example 6: Synthesis of Compound T-10

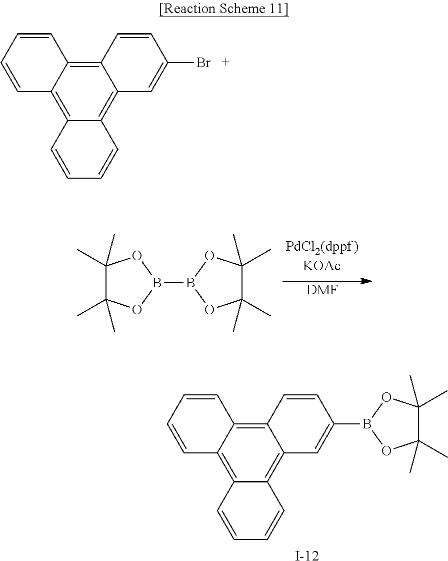

First Step: Synthesis of Intermediate I-12

2-bromotriphenylene (100 g, 326 mmol) was dissolved in 1 L of DMF in an nitrogen environment, bis(pinacolato)diboron (99.2 g, 391 mmol), (1,1′-bis(diphenylphosphine)ferrocene)dichloropalladium (II) (2.66 g, 3.26 mmol), and potassium acetate (80 g, 815 mmol) were added thereto, and the mixture was heated and refluxed at 150° C. for 5 hours. When the reaction was complete, water was added to the reaction solution, and the mixture was filtered and dried in a vacuum oven. This obtained residue was separated and purified through flash column chromatography to obtain Intermediate I-12 (113 g, 98%).

HRMS (70 eV, EI+): m/z calcd for C24H23BO2: 354.25, found: 354.

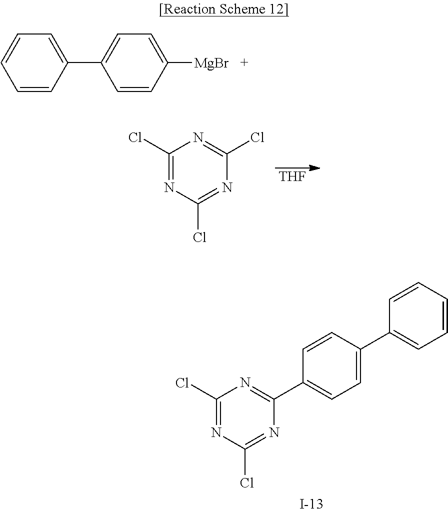

Second Step: Synthesis of Intermediate I-13

4-bromo-1,1′-biphenyl (11.8 mL, 47 mmol) and Mg (4.0 g, 164.6 mmol) were added to 30 mL of tetrahydrofuran (THF) in a nitrogen environment, and the mixture was refluxed for 3 hours. The prepared [1,1′-biphenyl]-4-yl magnesium bromide solution was slowly added in a dropwise fashion to a solution obtained by dissolving 2,4,6-trichloro-1,3,5-triazine (8.3 g, 44.7 mmol) in 80 mL of THF at 0° C. The obtained mixture was slowly heated up to room temperature and stirred for 12 hours. When the reaction was complete, the resultant was quenched with a 10% HCl aqueous solution, extracted with dichloromethane (DCM), and treated with anhydrous MgSO₄ to remove moisture, filtered, and concentrated under a reduced pressure. This obtained residue was separated and purified through flash column chromatography to obtain Intermediate I-13 (10.4 g, 73%).

HRMS (70 eV, EI+): m/z calcd for C15H19Cl2N3: 302.16, found: 302.

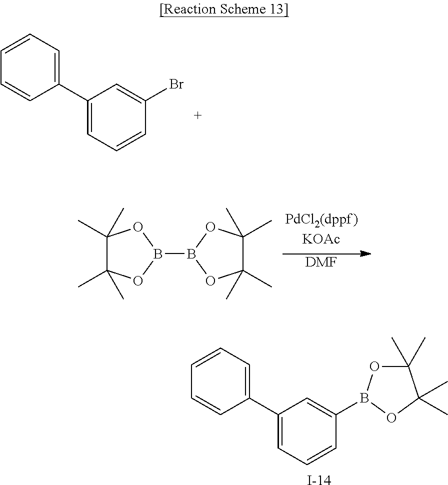

Third Step: Synthesis of Intermediate I-14

3-bromo-1,1′-biphenyl (29.7 g, 127.4 mmol) was dissolved in 500 mL of DMF in an nitrogen environment, bis(pinacolato)diboron (42.0 g, 165.4 mmol), ((1,1′-bis(diphenylphosphine)ferrocene)dichloropalladium (II)) (5.2 g, 6.36 mmol), and potassium acetate (18.7 g, 190.9 mmol) were added thereto, and the mixture was heated and refluxed at 120° C. for 8 hours. When the reaction was complete, water was added to the reaction solution, and the mixture was filtered and dried in a vacuum oven. This obtained residue was separated and purified through flash column chromatography to obtain Intermediate I-14 (30.3 g, 85%).

HRMS (70 eV, EI+): m/z calcd for C18H21BO2: 280.17, found 280.

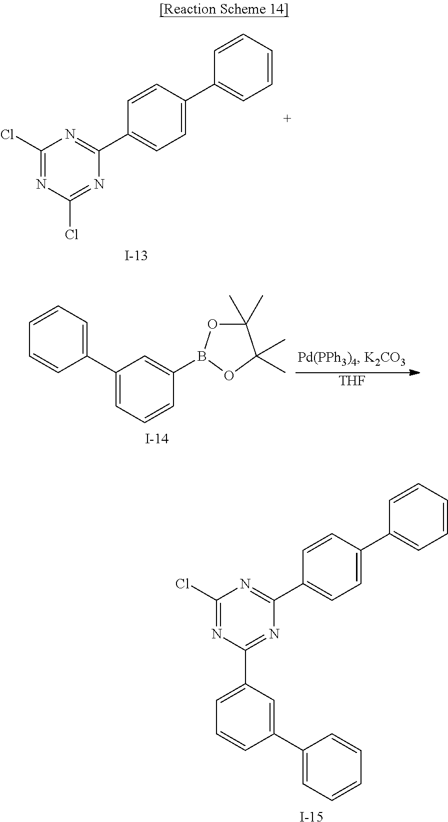

Fourth Step: Synthesis of Intermediate I-15

The Intermediate I-13 (10.3 g, 34 mmol) was dissolved in 200 mL of THF in a nitrogen environment, the Intermediate I-14 (9.5 g, 34 mmol) and tetrakis(triphenylphosphine)palladium (Pd(PPh₃)₄) (2.0 g, 1.7 mmol) were added thereto, and the mixture was stirred. 50 mL of a solution of potassium carbonate saturated in water (K₂CO₃, 9.4 g, 68 mmol) was added thereto, and the obtained mixture was heated and refluxed at 80° C. for 12 hours. When the reaction was completed, water of the reaction solution was extracted, and the solvent was removed using a rotary evaporator. This obtained residue was extracted with DCM, recrystallized and purified with a mixed solution of DCM: n-hexane to obtain Intermediate I-15 (11 g, 77%).

HRMS (70 eV, EI+): m/z calcd for C27H18ClN3: 419.91, found 419.

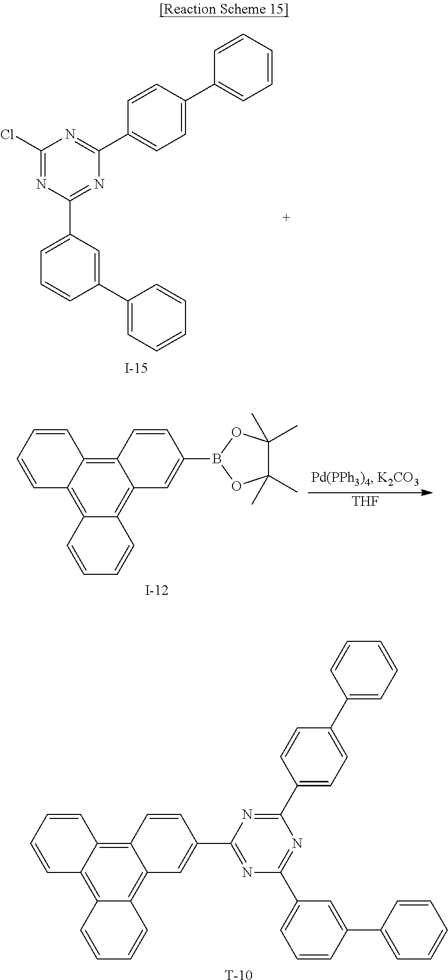

Fifth Step: Synthesis of Compound T-10

The Intermediate I-15 (11 g, 36.4 mmol) was dissolved in 200 mL of THF in an nitrogen environment, the Intermediate I-12 (12.9 g, 36.4 mmol) and tetrakis(triphenylphosphine)palladium (Pd(PPh₃)₄) (2.1 g, 1.82 mmol) were added thereto, and the mixture was stirred. 50 mL of a solution of potassium carbonate saturated in water (K₂CO₃, 10.1 g, 72.8 mmol) was added thereto, and the mixture was heated and refluxed at 80° C. for 12 hours. When the reaction was completed, water of the reaction solution was extracted, and the solvent was removed using a rotary evaporator. This obtained residue was once extracted with DCM and then, recrystallized and purified with a mixed solution of DCM: n-hexane to obtain Compound T-10 (15.8 g, 71%).

HRMS (70 eV, EI+): m/z calcd for C45H29N3: 611.73, found 611.

### Synthesis Example 7: Synthesis of Compound T-11

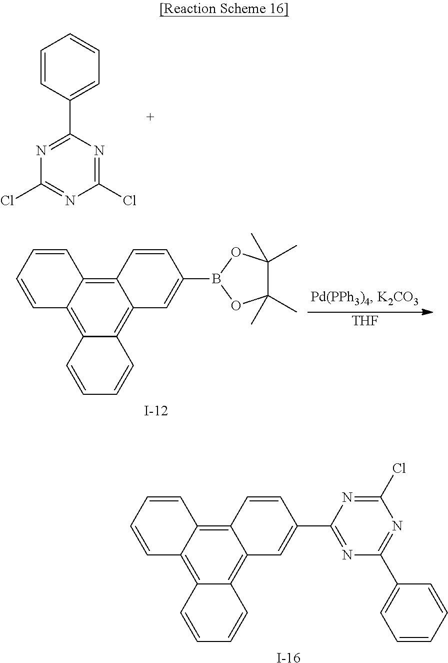

First Step: Synthesis of Intermediate I-16

Intermediate I-16 (14.3 g, 80%) was obtained under the same reaction condition as the fifth step of Synthesis Example 6 by using 2,4-dichloro-6-phenyl-1,3,5-triazine instead of the Intermediate I-15.

HRMS (70 eV, EI+): m/z calcd for C30H19Cl: 414.93, found 414.

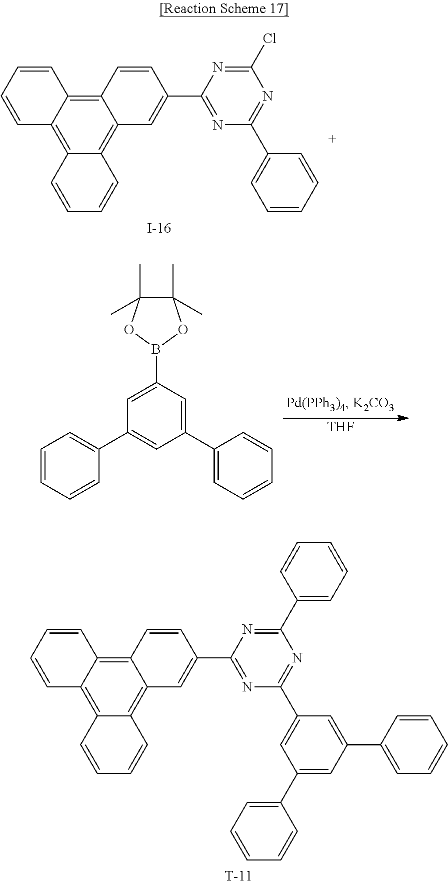

Second Step: Synthesis of Compound T-11

Compound T-11 (14.1 g, 79%) was obtained under the same reaction condition as the fifth step of Synthesis Example 6 by reacting the Intermediate I-16 (9.7 g, 43.1 mmol) and 2-([1,1′:3′,1″-terphenyl]-5′-yl)-4,4,5,5-tetramethyl-1,3,2-dioxaborolane in an nitrogen environment.

HRMS (70 eV, EI+): m/z calcd for C30H19Cl: 414.93, found 414.

### Synthesis Example 8: Synthesis of Compound T-12

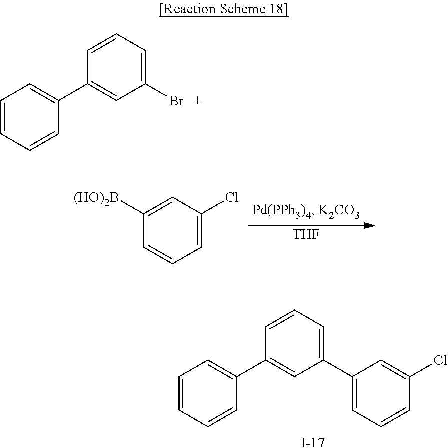

First Step: Synthesis of Intermediate I-17

3-bromobiphenyl (100 g, 429 mmol) was dissolved in a 850 mL of mixed solution THF:1,4-dioxane (a ratio of 1:1 ratio) in an nitrogen environment, 3-chlorophenylboronic acid (93.9 g, 601 mmol) and tetrakis(triphenylphosphine)palladium (Pd(PPh₃)₄) (24.8 g, 21 mmol) were added thereto, and the mixture was stirred. 500 mL of a solution of potassium carbonate saturated in water (K₂CO₃, 148.2 g, 1.07 mol) was added thereto, and the obtained mixture was heated and refluxed at 80° C. for 12 hours. When the reaction was completed, water of the reaction solution was extracted, and the solvent was all removed using a rotary evaporator. This obtained residue was once extracted with DCM and then, separated and purified through silica gel column chromatography to obtain Intermediate I-17 (106.0 g, 93%).

HRMS (70 eV, EI+): m/z calcd for C18H13Cl: 264.75, found 264.

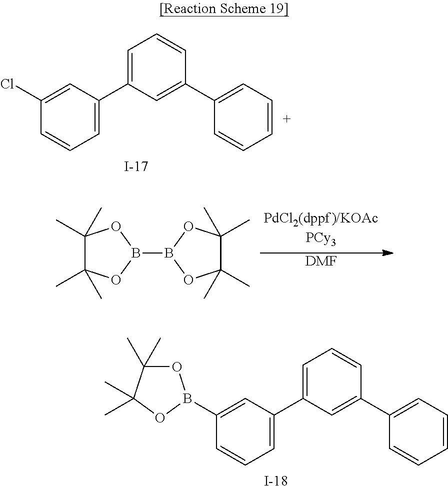

Second Step: Synthesis of Intermediate I-18

The Intermediate I-17 (36 g, 136 mmol) was dissolved in 1 L of dimethylformamide (DMF) in an nitrogen environment, bis(pinacolato)diboron (43.2 g, 170 mmol) and 1,1′-bis(diphenylphosphine)ferrocene)dichloropalladium (II) (Pd(dppf) (4.4 g, 5 mmol), tricyclohexyl phosphine (4.6 g, 16 mmol), and potassium acetate (KOAc) (40.0 g, 408 mmol) were added thereto, and the mixture was heated and refluxed at 140° C. for 12 hours. When the reaction was completed, water was added thereto to precipitate a solid, and the resultant was twice extracted with DCM after filtering the solid. This obtained residue was separated and purified through silica gel column chromatography to obtain Intermediate I-18 (20.0 g, 41.3%).

HRMS (70 eV, EI+): m/z calcd for C24H25BO2: 356.27, found 356.

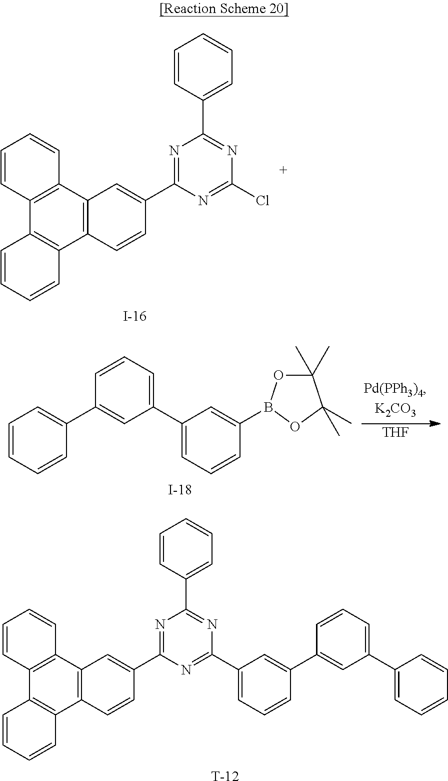

Third Step: Synthesis of Compound T-12

The Intermediate I-16 (7.4 g, 18 mmol) and the Intermediate I-18 (6.9 g, 19 mmol) were obtained under the same reaction condition as the fifth step of Synthesis Example 6 in an nitrogen environment to obtain Compound T-12 (6.4 g, 59.3%).

HRMS (70 eV, EI+): m/z calcd for C45H2N3: 611.73, found 611.

### Synthesis Example 9: Synthesis of Compound T-38

Compound T-38 was synthesized according to a synthesis method of Compound A-33 of Synthesis Example 17 in Patent Laid Open KR 10-2015-0028579.

### Synthesis Example 10: Synthesis of Compound T-79

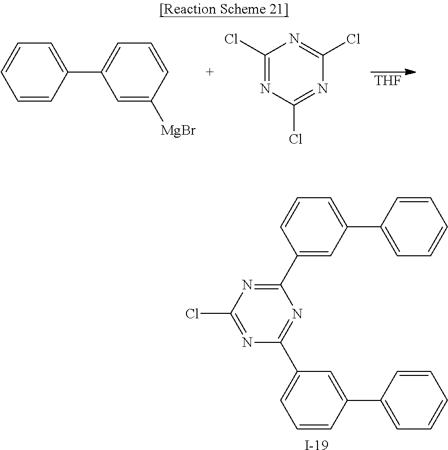

First Step: Synthesis of Intermediate I-19

Intermediate I-19 was synthesized according to a synthesis of Compound 5 in Patent Laid Open US 2015-0349268.

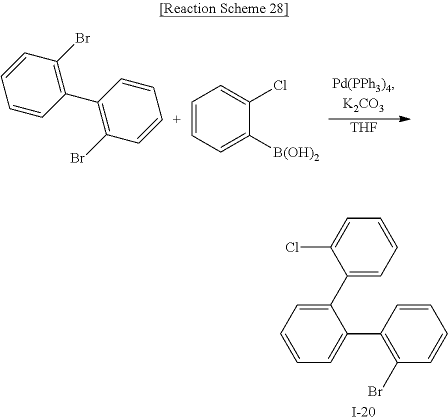

Second Step: Synthesis of Intermediate I-20

2,2′-dibromo-1,1′-biphenyl (79.9 g, 256 mmol) was dissolved in 1 L of tetrahydrofuran (THF) in an nitrogen environment, (2-chlorophenyl)boronic acid (36.4 g, 232.8 mmol) and tetrakis(triphenylphosphine)palladium (Pd(PPh₃)₄) (13.5 g, 11.6 mmol) were added thereto, and the mixture was stirred. Potassium carbonate saturated in water (K₂CO₃, 64.4 g, 465.6 mmol) was added thereto, and the obtained mixture was heated and refluxed at 80° C. for 12 hours. When the reaction was complete, water was added to the reaction solution, and the mixture was extracted with dichloromethane (DCM), treated with anhydrous MgSO₄ to remove moisture, filtered, and concentrated under a reduced pressure. This obtained residue was separated and purified through flash column chromatography to obtain Intermediate I-20 (62 g, 78%).

HRMS (70 eV, EI+): m/z calcd for C18H12BrCl: 343.65, found 343.

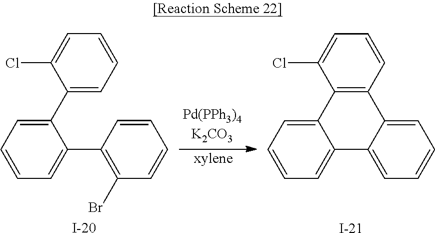

Third Step: Synthesis of Intermediate I-21

The Intermediate I-20 (62 g, 178.9 mmol) was dissolved in 600 mL of xylene in an nitrogen environment, tetrakis(triphenylphosphine)palladium (Pd(PPh₃)₄) (10.3 g, 8.9 mmol) and potassium carbonate (K₂CO₃, 32.1 g, 232.6 mmol) were added thereto, and the mixture was heated and refluxed for 10 hours. When the reaction was complete, ethylacetate and distilled water were used for an extraction, and an organic layer was treated with MgSO₄ to remove moisture, filtered, and concentrated under a reduced pressure. A product therefrom was purified with n-hexane/dichloromethane (7:3 of a volume ratio) through silica gel column chromatography to obtain a desired compound, Intermediate I-21 (11 g, 23%).

HRMS (70 eV, EI+): m/z calcd for C18H11Cl: 262.73, found 263.

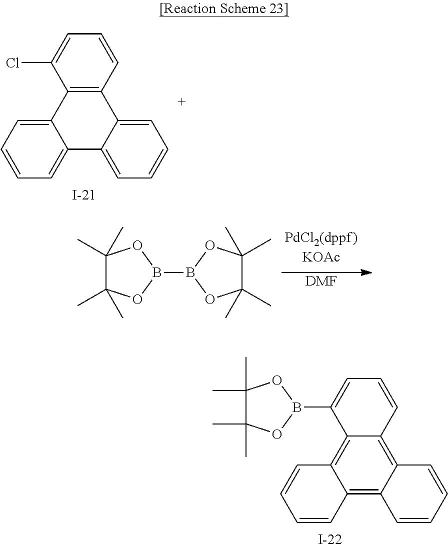

Fourth Step: Synthesis of Intermediate I-22

The Intermediate I-21 (22.9 g, 87.2 mmol) was dissolved in 500 mL of dimethylformamide (DMF) in an nitrogen environment, bis(pinacolato)diboron (28.8 g, 113.3 mmol), (1,1′-bis(diphenylphosphine)ferrocene)dichloropalladium (II) (PdCl₂(dppf)) (3.6 g, 4.4 mmol), and potassium acetate (KOAc) (12.8 g, 130.8 mmol) were added thereto, and the mixture was heated and refluxed at 140° C. for 12 hours. When the reaction was completed, water was added to the reaction solution to precipitate a solid, and DCM was twice used for an extraction after filtering the solid. This obtained residue was separated and purified through silica gel column chromatography to obtain Intermediate I-22 (21.0 g, 68%).

HRMS (70 eV, EI+): m/z calcd for C24H25BO2: 354.25, found 354.

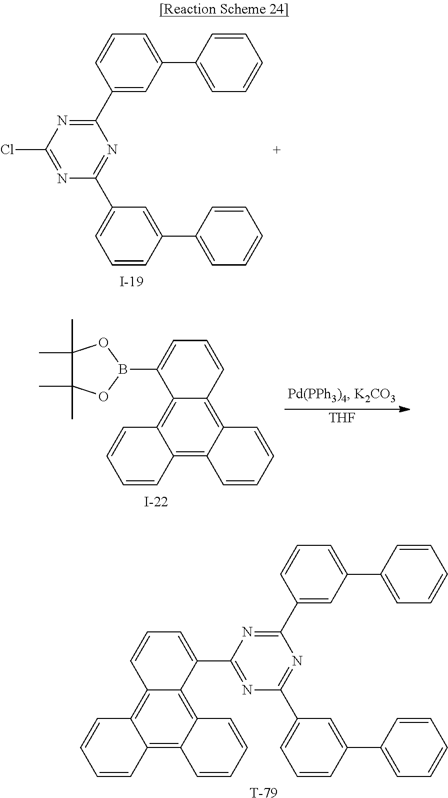

Fifth Step: Synthesis of Compound T-79

Compound T-79 (11.2 g, 65%) was obtained under the same reaction condition as the fifth step of Synthesis Example 6 by using the Intermediate I-19 (11.8 g, 28.2 mmol) and the Intermediate I-22 (9.5 g, 26.8 mmol) in an nitrogen environment.

HRMS (70 eV, EI+): m/z calcd for C₄₅H₂₉N₃: 611.73, found 611.

Manufacture of Organic Light Emitting Diode

### Example 1

ITO (indium tin oxide) was coated to be 1500 Å thick on a glass substrate, and the coated glass was ultrasonic wave-washed with a distilled water. After washing with the distilled water, the glass substrate was ultrasonic wave-washed with a solvent such as isopropyl alcohol, acetone, methanol, and the like and dried and then, moved to a plasma cleaner, cleaned by using oxygen plasma for 10 minutes, and moved to a vacuum depositor. This obtained ITO transparent electrode was used as an anode, a 700 Å-thick hole injection layer was formed on the ITO substrate by vacuum depositing N4,N4′-diphenyl-N4,N4′-bis (9-phenyl-9H-carbazol-3-yl)biphenyl-4,4′-diamine (Compound A), and a hole transport layer was formed by depositing 1,4,5,8,9,11-hexaazatriphenylene-hexacarbonitrile (HAT-CN) (Compound B) in a thickness of 50 Å on the injection layer, and depositing N-(biphenyl-4-yl)-9,9-dimethyl-N-(4-(9-phenyl-9H-carbazol-3-yl)phenyl)-9H-fluoren-2-amine (Compound C) in a thickness of 1020 Å. On the hole transport layer, a 400 Å-thick emission layer was formed by vacuum-depositing Compound T-9 and Compound E-1 as a host and tris(4-methyl-2,5-diphenylpyridine)iridium (III) (Compound D) as a dopant in a doping amount of 10 wt %

Herein, Compound T-9 and Compound E-1 were used in a ratio of 4:6.

Subsequently, a 300 Å-thick electron transport layer was formed by vacuum-depositing 8-(4-(4-(naphthalen-2-yl)-6-(naphthalen-3-yl)-1,3,5-triazin-2-yl)phenyl)quinoline (Compound E) and Liq simultaneously in a 1:1 ratio on the emission layer, and Liq (15 Å) and Al (1200 Å) were sequentially vacuum-deposited on the electron transport layer to form a cathode, manufacturing an organic light emitting diode.

The organic light emitting diode has five organic thin layers, specifically

ITO/A 700 Å/B 50 Å/C 1020 Å/EML [T-9:E-1:D=X:X:10%]400 Å/E:Liq 300 Å/Liq 15 Å/Al 1200 Å.

(X=a weight ratio)

### Examples 2 to 5

Organic light emitting diodes according to Examples 2 to 5 were manufactured by changing a mixing ratio of the first and second hosts in Example 1 as shown in Table 1.

### Comparative Example 1

An organic light emitting diode was manufactured according to the same method as

Example 1 except for using 4,4′-di(9H-carbazol-9-yl)biphenyl (CBP) as a single host instead of the two kinds of host.

### Comparative Example 2

An organic light emitting diode was manufactured according to the same method as Example 1 except for using Compound T-38 as a single host instead of the two kinds of host.

### Comparative Example 3

An organic light emitting diode was manufactured according to the same method as Example 1 except for using Compound HH-1 instead of the first host and Compound EH-1 instead of the second host in a ratio of 5:5.

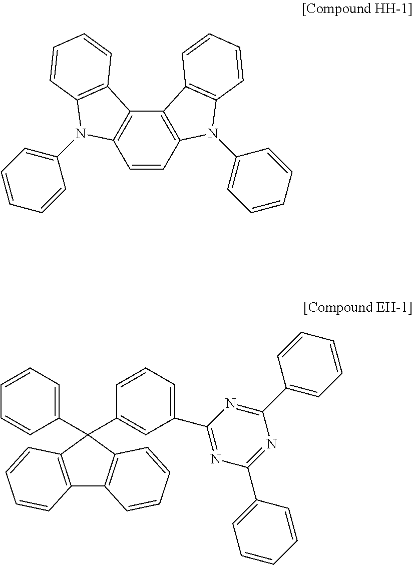 [SMILES: c1ccc(-n2c3ccccc3c3c4c5ccccc5n(-c5ccccc5)c4ccc32)cc1 ; c1ccc(-c2nc(-c3ccccc3)nc(-c3cccc(C4(c5ccccc5)c5ccccc5-c5ccccc54)c3)n2)cc1]

Evaluation

Luminous efficiency and life-span characteristics of the organic light emitting diodes according to Examples 1 to 5 and Comparative Examples 1 to 3 were evaluated.

Specific measurement methods are as follows, and the results are shown in Table 1.

(1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

(2) Measurement of Luminance Change Depending on Voltage Change

Luminance was measured by using a luminance meter (Minolta Cs-1000A), while the voltage of the organic light emitting diodes was increased from 0 V to 10 V.

(3) Measurement of Luminous Efficiency

Current efficiency (cd/A) at the same current density (10 mA/cm²) were calculated by using the luminance, current density, and voltages (V) from the items (1) and (2).

(4) Roll-Off Measurement

Roll-off was measured by calculating the falling amount of efficiency as % according to (Max measurement−Measurement at 6000 cd/m²/Max measurement) from the characteristic measurements of the (3).

(5) Measurement of Life-Span

Life-span was obtained by measuring time taken until current efficiency (cd/A) decreased down to 97% while luminance (cd/m²) was maintained at 6000 cd/m².

TABLE 1

| | | | First | | Light | | Life- |
|---|---|---|---|---|---|---|---|
| | | | host:Second | Driving | emitting | Roll- | span |
| | First | Second | host | voltage | efficiency | off | T97 |
| | host | host | (wt/wt) | (V) | (cd/A) | (%) | (h) |
| Example 1 | E-1 | T-9 | 6:4 | 3.9 | 62.4 | 7.9 | 210 |
| Example 2 | E-2 | T-9 | 6:4 | 3.8 | 69.3 | 6.6 | 280 |
| Example 3 | E-1 | T-38 | 5:5 | 4.1 | 68.0 | 2.4 | 140 |
| Example 4 | E-2 | T-38 | 5:5 | 4.0 | 68.8 | 7.7 | 230 |
| Example 5 | C-2 | T-38 | 5:5 | 4.0 | 69.2 | 4.1 | 120 |
| Comparative | CBP | — | — | — | 19.3 | 0.9 | 0.5 |
| Example 1 | | | | | | | |
| Comparative | T-38 | — | — | 4.8 | 44.8 | 12.9 | 20 |
| Example 2 | | | | | | | |
| Comparative | HH-1 | EH-1 | 5:5 | 5.6 | 30.5 | — | — |
| Example 3 | | | | | | | |

* A life-span of a device having luminance of less than or equal to 6000 cd/m² is immeasurable

Referring to Table 1, the organic light emitting diodes according to Examples 1 to 5 simultaneously showed remarkably improved driving voltage, luminous efficiency, roll-off characteristics and life-span characteristics compared with the organic light emitting diodes according to Comparative Examples 1 to 3.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims. Therefore, the aforementioned embodiments should be understood to be exemplary but not limiting the present invention in any way.

### DESCRIPTION OF SYMBOLS

100, 200: organic light emitting diode
105: organic layer
110: cathode
120: anode
130: emission layer
140: hole auxiliary layer

What is claimed is:

1. A composition for an organic optoelectric device, comprising
at least one of a first host compound represented by a combination of Chemical Formula 1 and Chemical Formula 2, and
at least one of a second host compound represented by Chemical Formula 3: 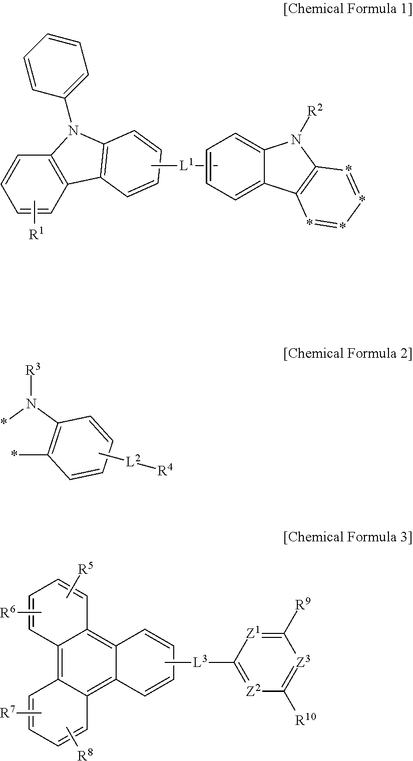
wherein, in Chemical Formulae 1 to 3,
adjacent two *'s of Chemical Formula 1 are linked with two *'s of Chemical Formula 2, and remaining *'s that are not linked with * of Chemical Formula 2 are independently CR^{a},
R¹, R⁴, and R^{a} are independently hydrogen, deuterium, a substituted or unsubstituted C6 to C30 aryl group, or a combination thereof,
R² and R³ are independently a substituted or unsubstituted C6 to C30 aryl group,
L¹ and L² are independently a single bond, or a substituted or unsubstituted phenylene group,
Z¹ to Z³ are independently CR^{b} or N,
at least one of Z¹ to Z³ is N,
R⁵ to R¹⁰ and R^{b} are independently hydrogen, deuterium, a substituted or unsubstituted C1 to C10 alkyl group, a substituted or unsubstituted C6 to C12 aryl group, a substituted or unsubstituted C2 to C12 heteroaryl group, or a combination thereof, and
L³ is a single bond, a substituted or unsubstituted phenylene group, a substituted or unsubstituted biphenylene group or a substituted or unsubstituted terphenylene group,
wherein “substituted” refers to replacement of at least one hydrogen by deuterium, a C1 to C4 alkyl group, or a C6 to C12 aryl group.

2. The composition for an organic optoelectric device of claim 1, wherein the first host compound is represented by Chemical Formula 1-A, 1-B, 1-C, 1-D, 1-E, or 1-F: 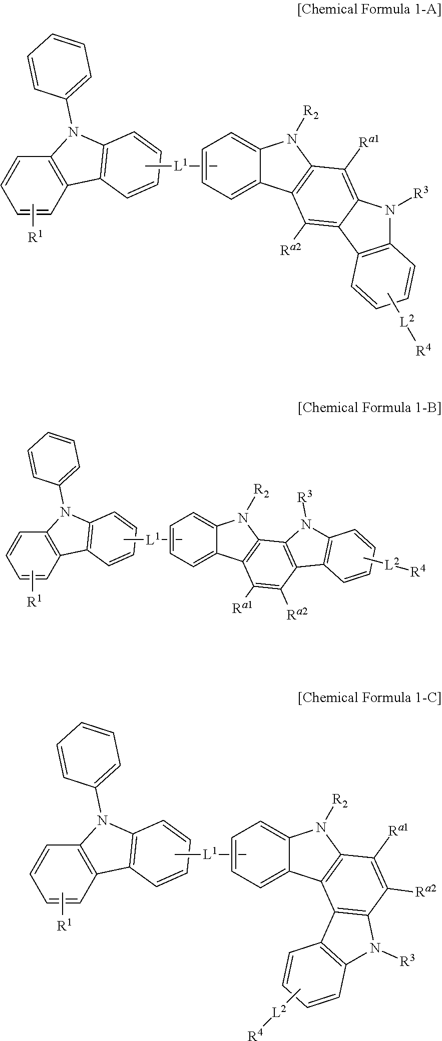 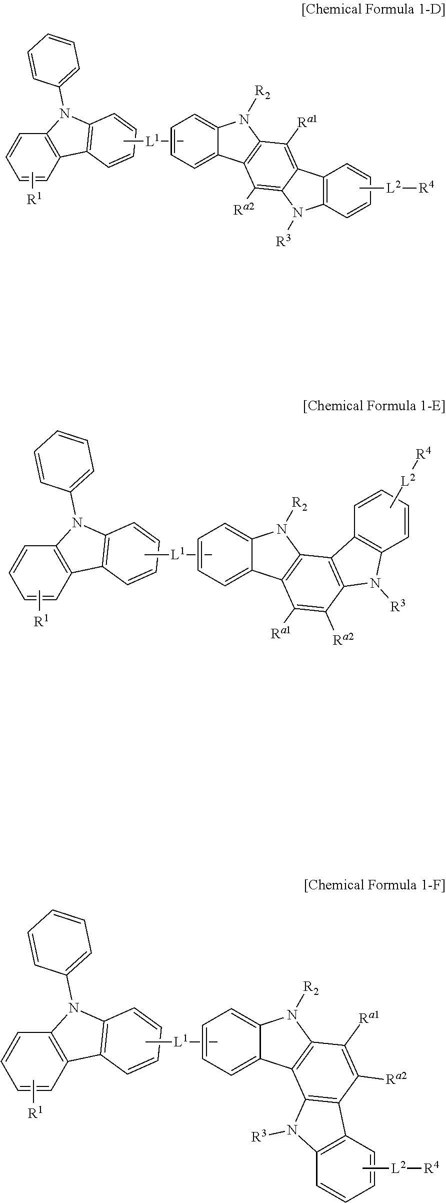
wherein, in Chemical Formulae 1-A to 1-F,
R¹, R⁴, R^{a1}, and R^{a2} are independently hydrogen, deuterium, a substituted or unsubstituted C6 to C30 aryl group, or a combination thereof,
R² and R³ are independently a substituted or unsubstituted C6 to C30 aryl group, and
L¹ and L² are independently a single bond, or a substituted or unsubstituted phenylene group.

3. The composition for an organic optoelectric device of claim 1, wherein Chemical Formula 1 is represented by Chemical Formula 1-I, 1-II, 1-IIII, or 1-IV: 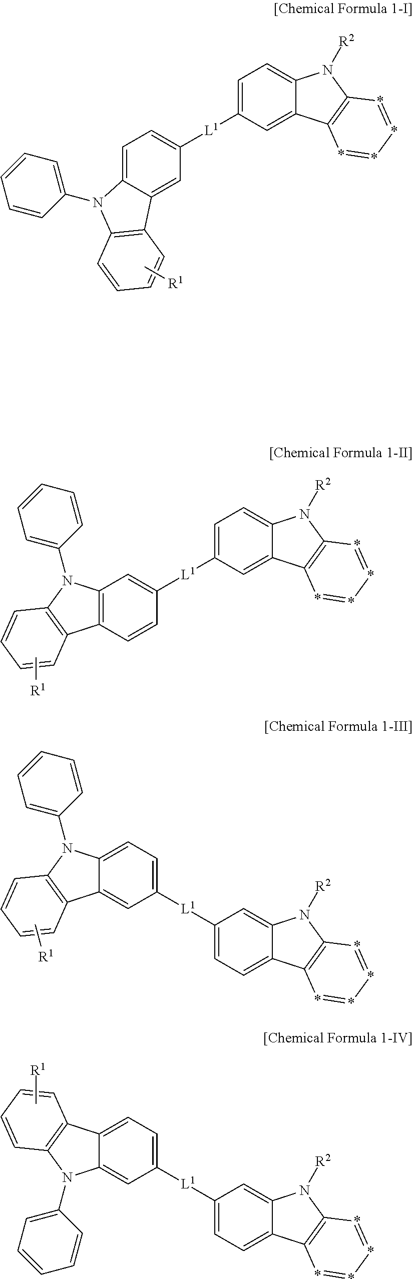
wherein, in Chemical Formulae 1-I to 1-IV,
adjacent two *'s of Chemical Formula 1 are linked with two *'s of Chemical Formula 2, and remaining *'s that are not linked with * of Chemical Formula 2 are independently CR^{a},
R¹ and R^{a} are independently hydrogen, deuterium, a substituted or unsubstituted C6 to C30 aryl group, or a combination thereof,
R² is a substituted or unsubstituted C6 to C30 aryl group, and
L¹ is a single bond, or a substituted or unsubstituted phenylene group.

4. The composition for an organic optoelectric device of claim 1, wherein the first host compound is represented by Chemical Formula 1-C1 or Chemical Formula 1-E1: 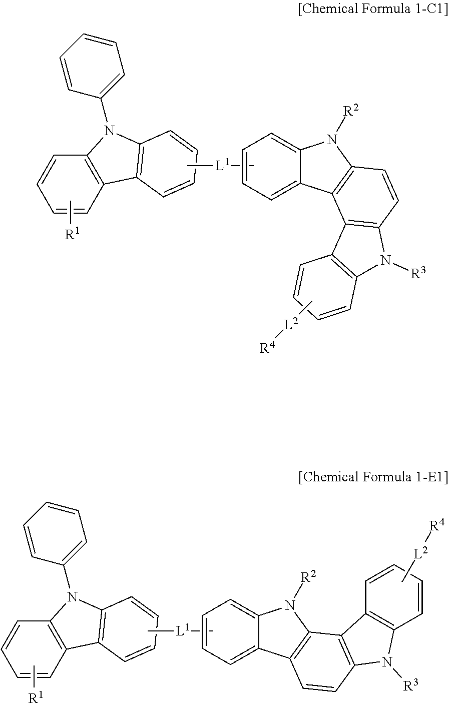
wherein, in Chemical Formulae 1-C1 and 1-E1,
R¹ and R⁴ are independently hydrogen, deuterium, a substituted or unsubstituted C6 to C18 aryl group, or a combination thereof,
R² and R³ are independently a substituted or unsubstituted C6 to C18 aryl group, and
L¹ and L² are independently a single bond, or a substituted or unsubstituted phenylene group.

5. The composition for an organic optoelectric device of claim 1, wherein R¹ and R⁴ are independently hydrogen, deuterium, a substituted or unsubstituted phenyl group, a substituted or unsubstituted biphenyl group, or a substituted or unsubstituted terphenyl group,
R² and R³ are independently a substituted or unsubstituted phenyl group, a substituted or unsubstituted biphenyl group, a substituted or unsubstituted terphenyl group, or a substituted or unsubstituted fluorenyl group.

6. The composition for an organic optoelectric device of claim 1, wherein the first host compound is selected from compounds of Group 1:
[Group 1]

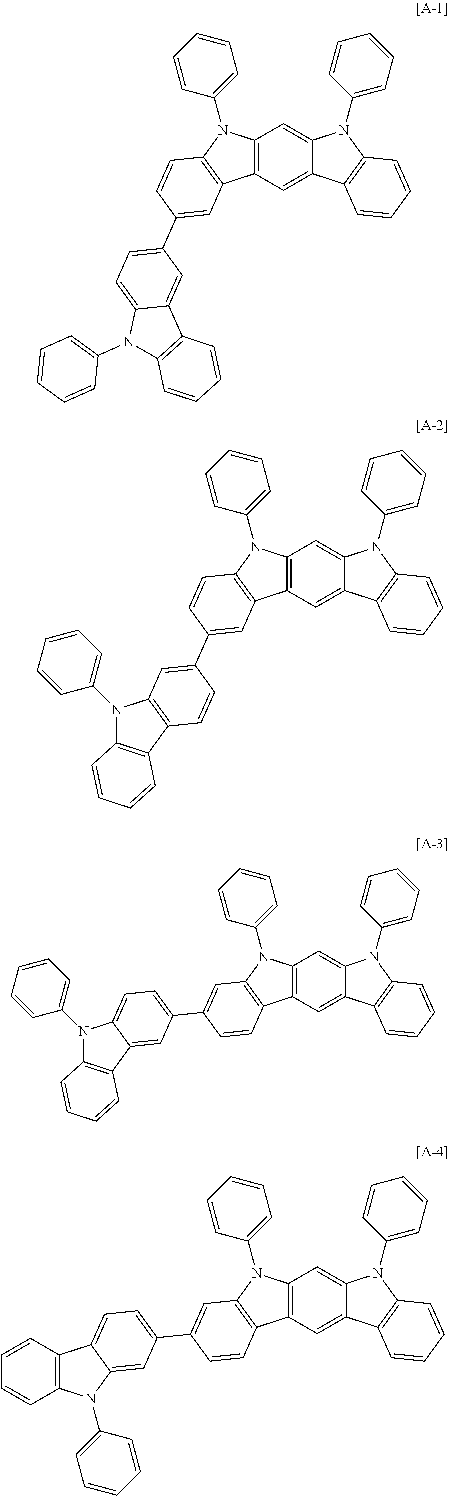 [SMILES: c1ccc(-n2c3ccccc3c3cc(-c4ccc5c(c4)c4cc6c7ccccc7n(-c7ccccc7)c6cc4n5-c4ccccc4)ccc32)cc1 ; c1ccc(-n2c3ccccc3c3ccc(-c4ccc5c(c4)c4cc6c7ccccc7n(-c7ccccc7)c6cc4n5-c4ccccc4)cc32)cc1 ; c1ccc(-n2c3ccccc3c3cc(-c4ccc5c6cc7c8ccccc8n(-c8ccccc8)c7cc6n(-c6ccccc6)c5c4)ccc32)cc1 ; c1ccc(-n2c3ccccc3c3ccc(-c4ccc5c6cc7c8ccccc8n(-c8ccccc8)c7cc6n(-c6ccccc6)c5c4)cc32)cc1]

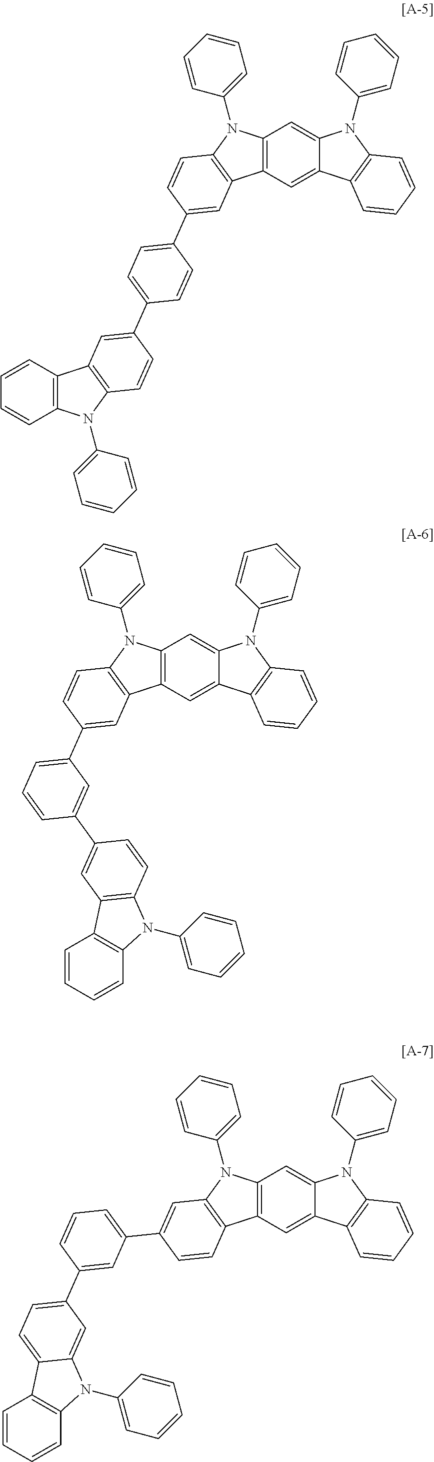

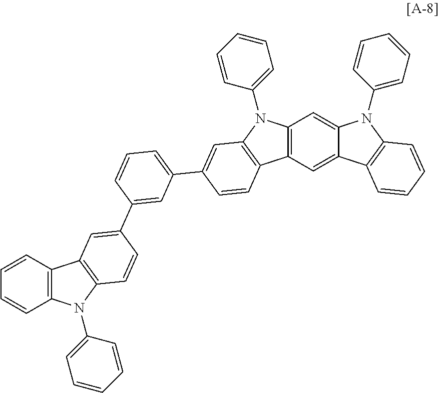 [SMILES: c1ccc(-n2c3ccccc3c3cc(-c4cccc(-c5ccc6c7cc8c9ccccc9n(-c9ccccc9)c8cc7n(-c7ccccc7)c6c5)c4)ccc32)cc1]

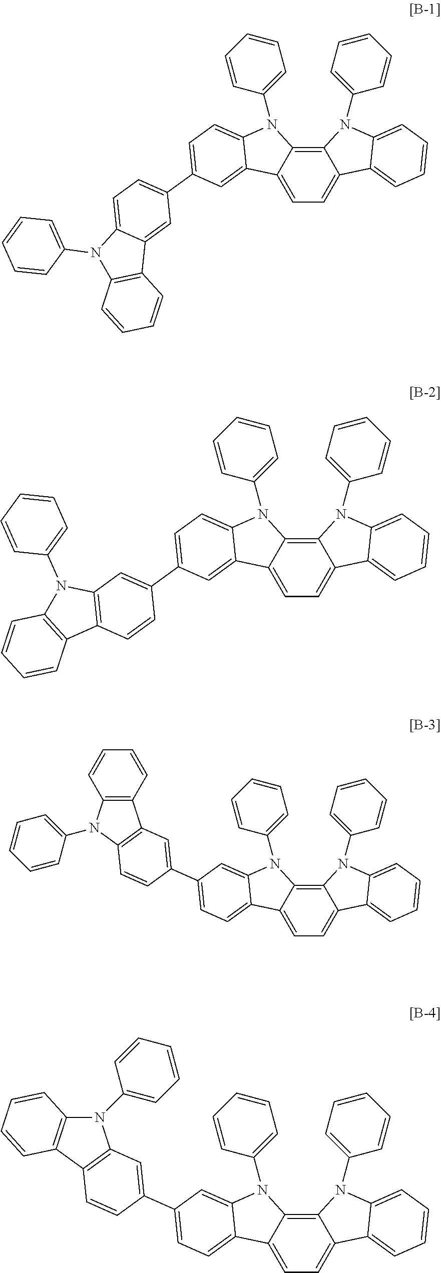 [SMILES: c1ccc(-n2c3ccccc3c3cc(-c4ccc5c(c4)c4ccc6c7ccccc7n(-c7ccccc7)c6c4n5-c4ccccc4)ccc32)cc1 ; c1ccc(-n2c3ccccc3c3ccc(-c4ccc5c(c4)c4ccc6c7ccccc7n(-c7ccccc7)c6c4n5-c4ccccc4)cc32)cc1 ; c1ccc(-n2c3ccccc3c3cc(-c4ccc5c6ccc7c8ccccc8n(-c8ccccc8)c7c6n(-c6ccccc6)c5c4)ccc32)cc1 ; c1ccc(-n2c3ccccc3c3ccc(-c4ccc5c6ccc7c8ccccc8n(-c8ccccc8)c7c6n(-c6ccccc6)c5c4)cc32)cc1]

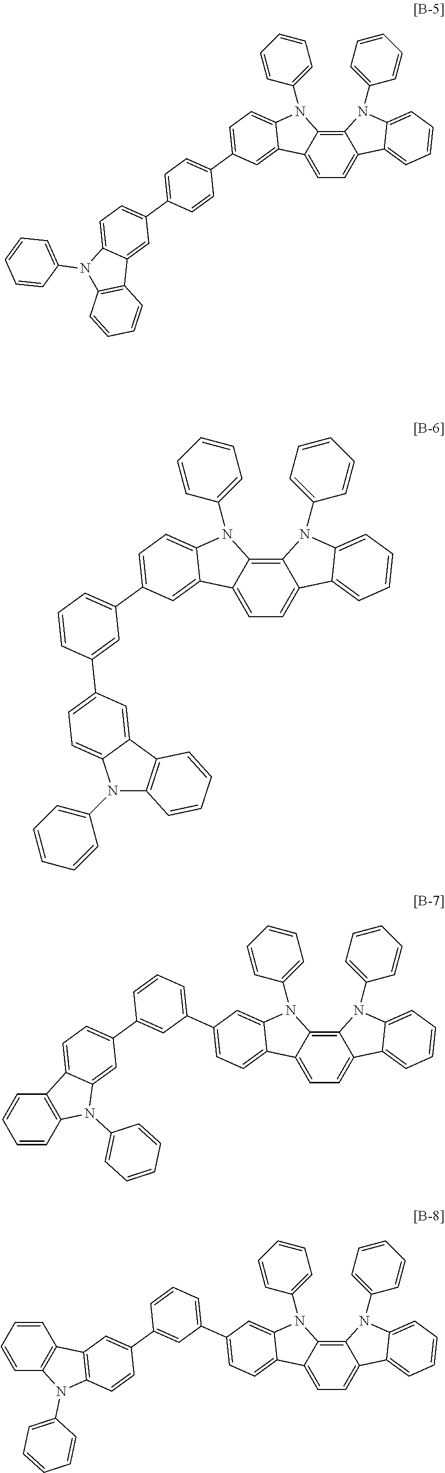 [SMILES: c1ccc(-n2c3ccccc3c3cc(-c4ccc(-c5ccc6c(c5)c5ccc7c8ccccc8n(-c8ccccc8)c7c5n6-c5ccccc5)cc4)ccc32)cc1 ; c1ccc(-n2c3ccccc3c3cc(-c4cccc(-c5ccc6c(c5)c5ccc7c8ccccc8n(-c8ccccc8)c7c5n6-c5ccccc5)c4)ccc32)cc1 ; c1ccc(-n2c3ccccc3c3ccc(-c4cccc(-c5ccc6c7ccc8c9ccccc9n(-c9ccccc9)c8c7n(-c7ccccc7)c6c5)c4)cc32)cc1 ; c1ccc(-n2c3ccccc3c3cc(-c4cccc(-c5ccc6c7ccc8c9ccccc9n(-c9ccccc9)c8c7n(-c7ccccc7)c6c5)c4)ccc32)cc1]

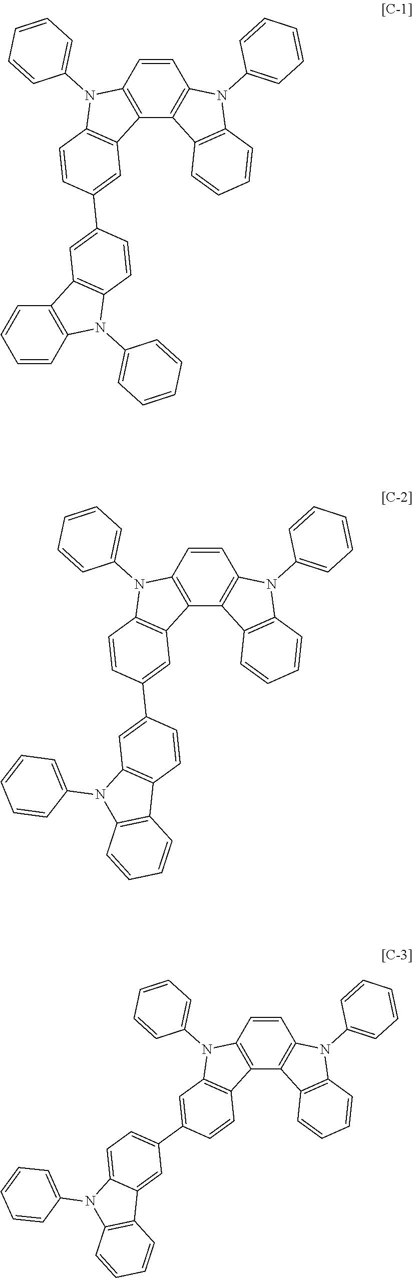 [SMILES: c1ccc(-n2c3ccccc3c3cc(-c4ccc5c(c4)c4c6c7ccccc7n(-c7ccccc7)c6ccc4n5-c4ccccc4)ccc32)cc1 ; c1ccc(-n2c3ccccc3c3ccc(-c4ccc5c(c4)c4c6c7ccccc7n(-c7ccccc7)c6ccc4n5-c4ccccc4)cc32)cc1 ; c1ccc(-n2c3ccccc3c3cc(-c4ccc5c6c7c8ccccc8n(-c8ccccc8)c7ccc6n(-c6ccccc6)c5c4)ccc32)cc1]

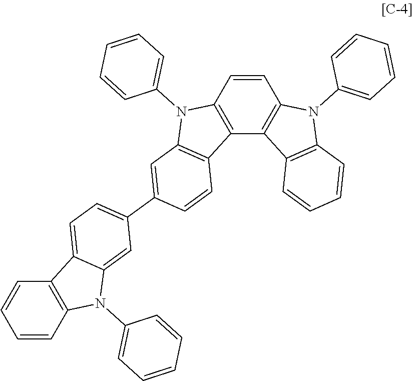 [SMILES: c1ccc(-n2c3ccccc3c3ccc(-c4ccc5c6c7c8ccccc8n(-c8ccccc8)c7ccc6n(-c6ccccc6)c5c4)cc32)cc1]

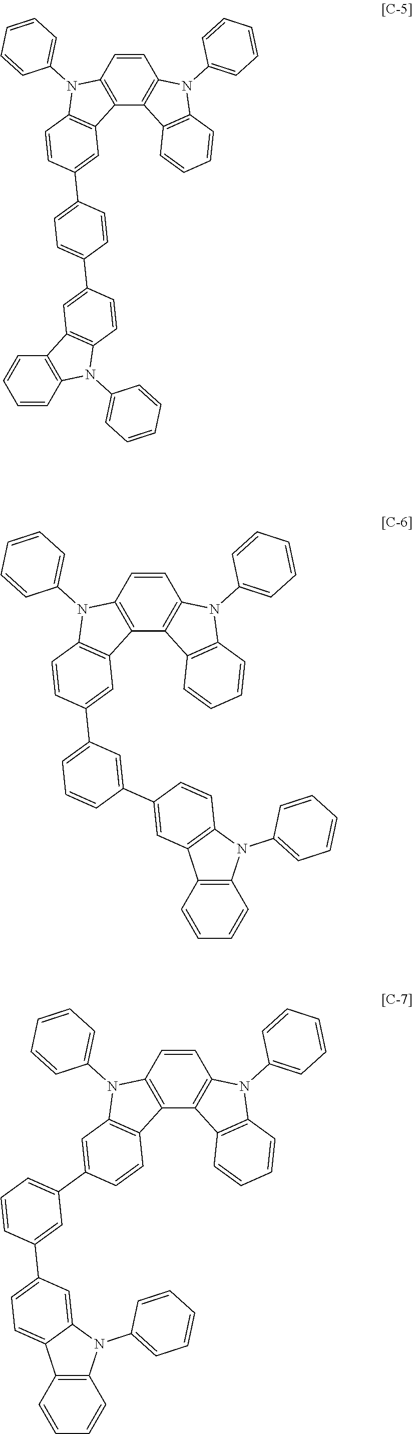 [SMILES: c1ccc(-n2c3ccccc3c3cc(-c4ccc(-c5ccc6c(c5)c5c7c8ccccc8n(-c8ccccc8)c7ccc5n6-c5ccccc5)cc4)ccc32)cc1 ; c1ccc(-n2c3ccccc3c3cc(-c4cccc(-c5ccc6c(c5)c5c7c8ccccc8n(-c8ccccc8)c7ccc5n6-c5ccccc5)c4)ccc32)cc1 ; c1ccc(-n2c3ccccc3c3ccc(-c4cccc(-c5ccc6c7c8c9ccccc9n(-c9ccccc9)c8ccc7n(-c7ccccc7)c6c5)c4)cc32)cc1]

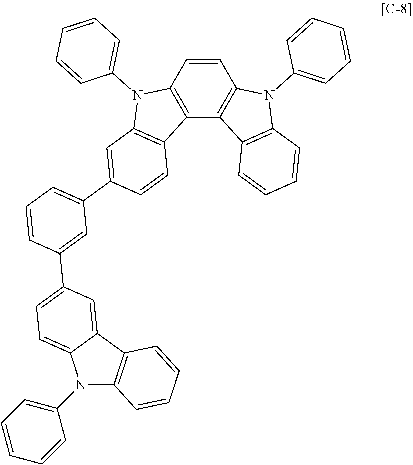 [SMILES: c1ccc(-n2c3ccccc3c3cc(-c4cccc(-c5ccc6c7c8c9ccccc9n(-c9ccccc9)c8ccc7n(-c7ccccc7)c6c5)c4)ccc32)cc1]

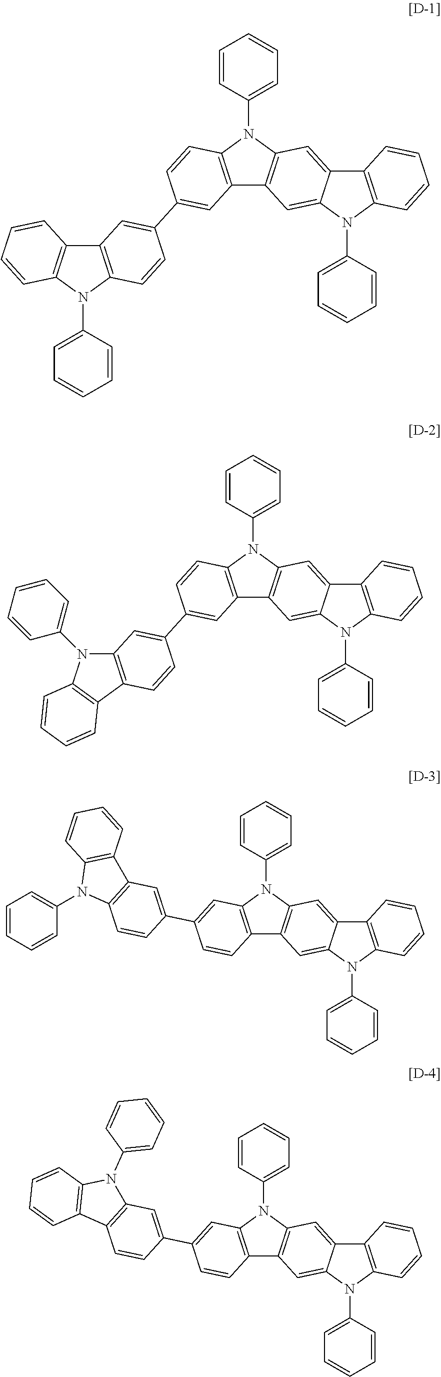 [SMILES: c1ccc(-n2c3ccccc3c3cc(-c4ccc5c(c4)c4cc6c(cc4n5-c4ccccc4)c4ccccc4n6-c4ccccc4)ccc32)cc1 ; c1ccc(-n2c3ccccc3c3ccc(-c4ccc5c(c4)c4cc6c(cc4n5-c4ccccc4)c4ccccc4n6-c4ccccc4)cc32)cc1 ; c1ccc(-n2c3ccccc3c3cc(-c4ccc5c6cc7c(cc6n(-c6ccccc6)c5c4)c4ccccc4n7-c4ccccc4)ccc32)cc1 ; c1ccc(-n2c3ccccc3c3ccc(-c4ccc5c6cc7c(cc6n(-c6ccccc6)c5c4)c4ccccc4n7-c4ccccc4)cc32)cc1]

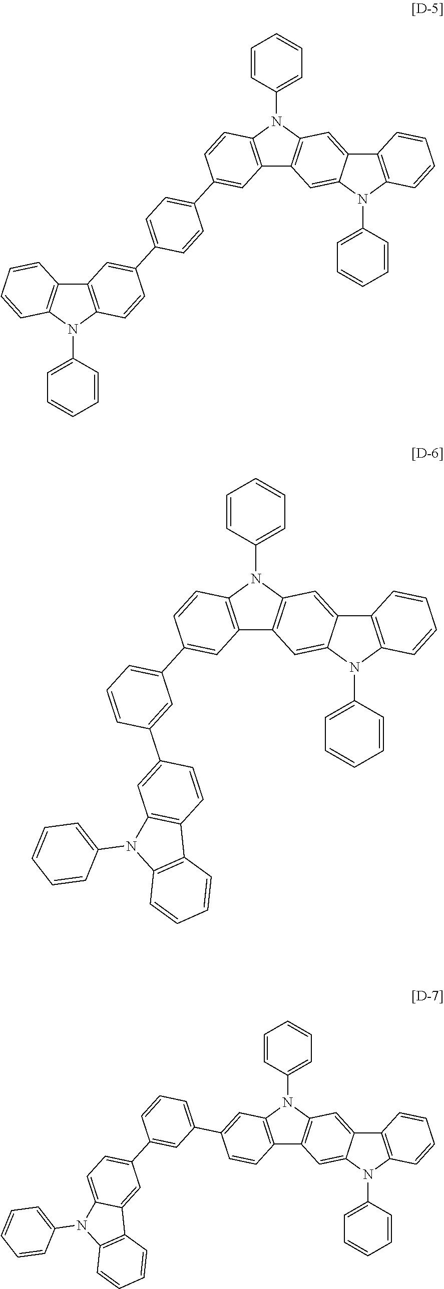 [SMILES: c1ccc(-n2c3ccccc3c3cc(-c4ccc(-c5ccc6c(c5)c5cc7c(cc5n6-c5ccccc5)c5ccccc5n7-c5ccccc5)cc4)ccc32)cc1 ; c1ccc(-n2c3ccccc3c3ccc(-c4cccc(-c5ccc6c(c5)c5cc7c(cc5n6-c5ccccc5)c5ccccc5n7-c5ccccc5)c4)cc32)cc1 ; c1ccc(-n2c3ccccc3c3cc(-c4cccc(-c5ccc6c7cc8c(cc7n(-c7ccccc7)c6c5)c5ccccc5n8-c5ccccc5)c4)ccc32)cc1]

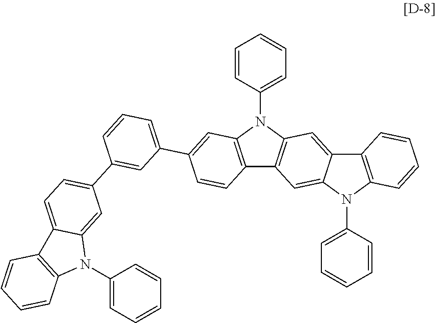 [SMILES: c1ccc(-n2c3ccccc3c3ccc(-c4cccc(-c5ccc6c7cc8c(cc7n(-c7ccccc7)c6c5)c5ccccc5n8-c5ccccc5)c4)cc32)cc1]

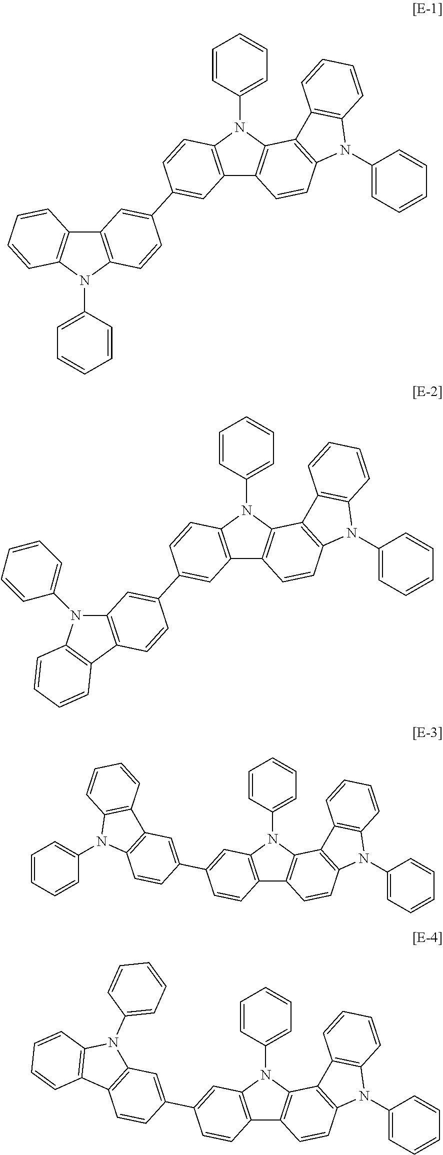 [SMILES: c1ccc(-n2c3ccccc3c3cc(-c4ccc5c(c4)c4ccc6c(c7ccccc7n6-c6ccccc6)c4n5-c4ccccc4)ccc32)cc1 ; c1ccc(-n2c3ccccc3c3ccc(-c4ccc5c(c4)c4ccc6c(c7ccccc7n6-c6ccccc6)c4n5-c4ccccc4)cc32)cc1 ; c1ccc(-n2c3ccccc3c3cc(-c4ccc5c6ccc7c(c8ccccc8n7-c7ccccc7)c6n(-c6ccccc6)c5c4)ccc32)cc1 ; c1ccc(-n2c3ccccc3c3ccc(-c4ccc5c6ccc7c(c8ccccc8n7-c7ccccc7)c6n(-c6ccccc6)c5c4)cc32)cc1]

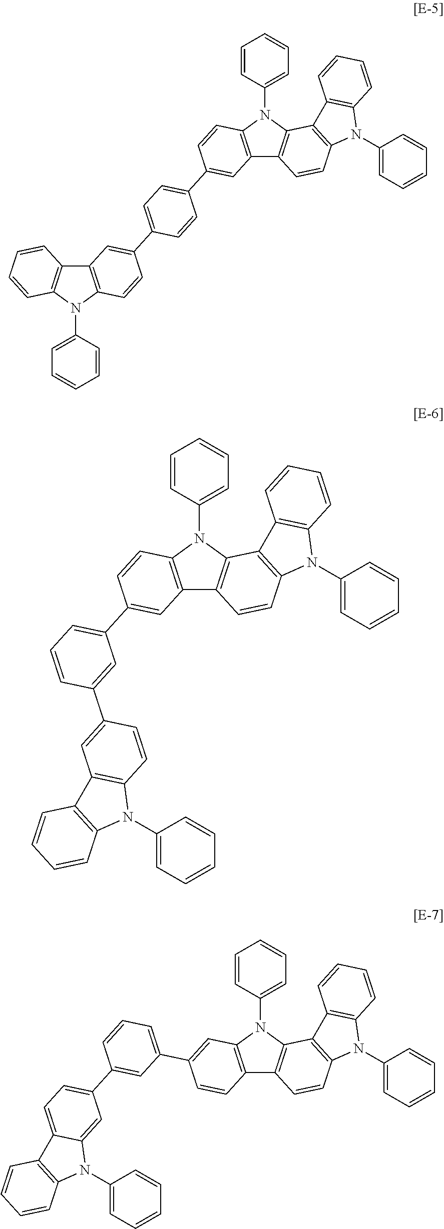 [SMILES: c1ccc(-n2c3ccccc3c3cc(-c4ccc(-c5ccc6c(c5)c5ccc7c(c8ccccc8n7-c7ccccc7)c5n6-c5ccccc5)cc4)ccc32)cc1 ; c1ccc(-n2c3ccccc3c3cc(-c4cccc(-c5ccc6c(c5)c5ccc7c(c8ccccc8n7-c7ccccc7)c5n6-c5ccccc5)c4)ccc32)cc1 ; c1ccc(-n2c3ccccc3c3ccc(-c4cccc(-c5ccc6c7ccc8c(c9ccccc9n8-c8ccccc8)c7n(-c7ccccc7)c6c5)c4)cc32)cc1]

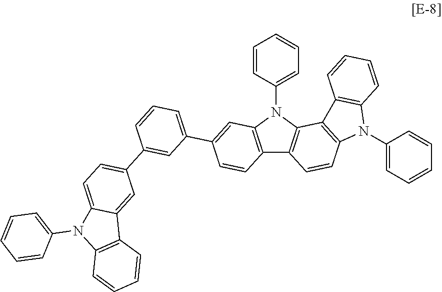 [SMILES: c1ccc(-n2c3ccccc3c3cc(-c4cccc(-c5ccc6c7ccc8c(c9ccccc9n8-c8ccccc8)c7n(-c7ccccc7)c6c5)c4)ccc32)cc1]

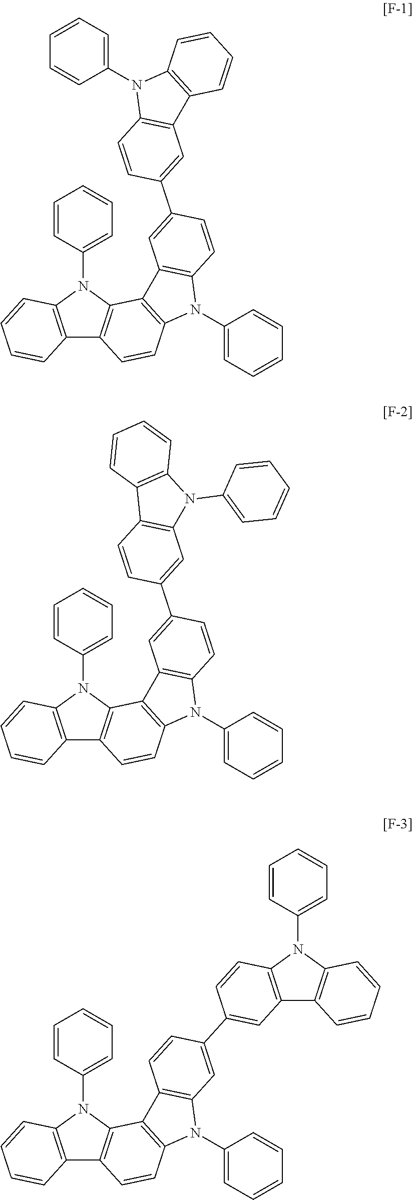 [SMILES: c1ccc(-n2c3ccccc3c3cc(-c4ccc5c(c4)c4c(ccc6c7ccccc7n(-c7ccccc7)c64)n5-c4ccccc4)ccc32)cc1 ; c1ccc(-n2c3ccccc3c3ccc(-c4ccc5c(c4)c4c(ccc6c7ccccc7n(-c7ccccc7)c64)n5-c4ccccc4)cc32)cc1 ; c1ccc(-n2c3ccccc3c3cc(-c4ccc5c6c(ccc7c8ccccc8n(-c8ccccc8)c76)n(-c6ccccc6)c5c4)ccc32)cc1]

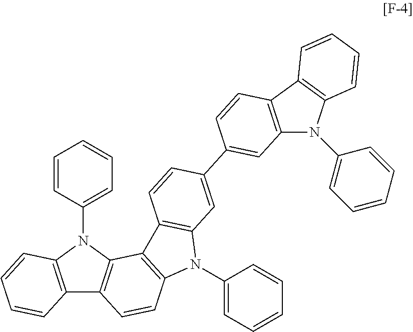 [SMILES: c1ccc(-n2c3ccccc3c3ccc(-c4ccc5c6c(ccc7c8ccccc8n(-c8ccccc8)c76)n(-c6ccccc6)c5c4)cc32)cc1]

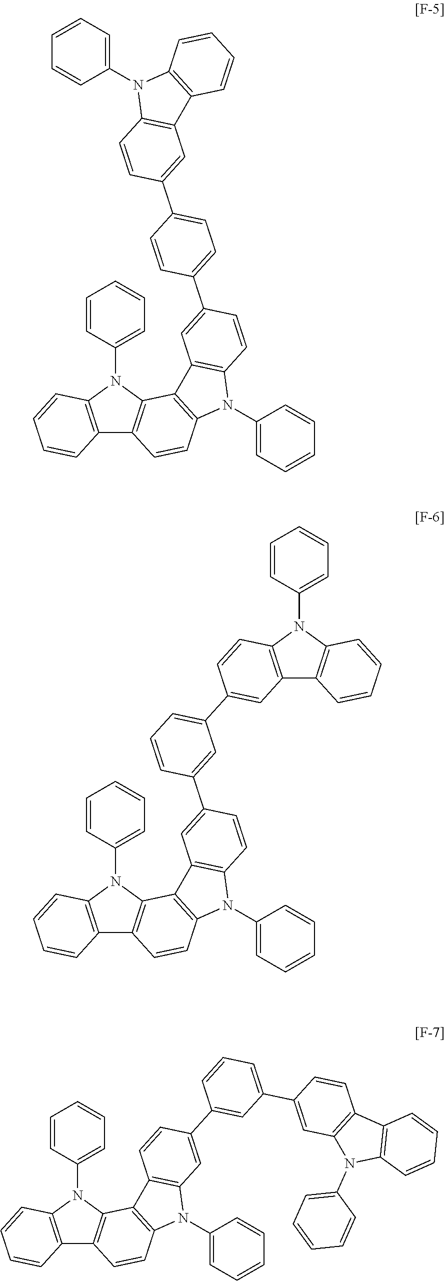 [SMILES: c1ccc(-n2c3ccccc3c3cc(-c4ccc(-c5ccc6c(c5)c5c(ccc7c8ccccc8n(-c8ccccc8)c75)n6-c5ccccc5)cc4)ccc32)cc1 ; c1ccc(-n2c3ccccc3c3cc(-c4cccc(-c5ccc6c(c5)c5c(ccc7c8ccccc8n(-c8ccccc8)c75)n6-c5ccccc5)c4)ccc32)cc1 ; c1ccc(-n2c3ccccc3c3ccc(-c4cccc(-c5ccc6c7c(ccc8c9ccccc9n(-c9ccccc9)c87)n(-c7ccccc7)c6c5)c4)cc32)cc1]

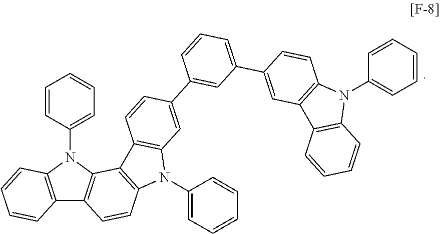 [SMILES: c1ccc(-n2c3ccccc3c3cc(-c4cccc(-c5ccc6c7c(ccc8c9ccccc9n(-c9ccccc9)c87)n(-c7ccccc7)c6c5)c4)ccc32)cc1]

7. The composition for an organic optoelectric device of claim 1, wherein the second host compound is represented by Chemical Formula 3-I or Chemical Formula 3-II: 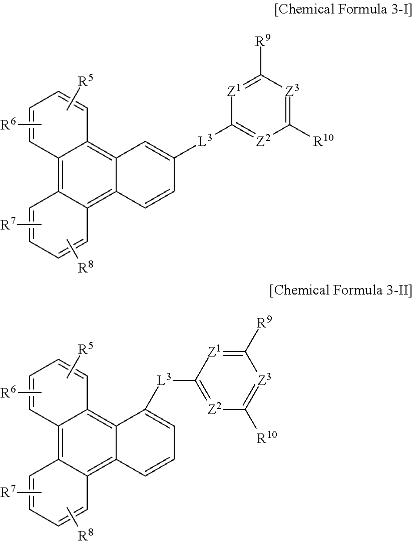
wherein, in Chemical Formulae 3-I and 3-II,
Z¹ to Z³ are independently CR^{b} or N,
at least one of Z¹ to Z³ is N,
R⁵ to R¹⁰ and R^{b} are independently hydrogen, deuterium, a substituted or unsubstituted C1 to C10 alkyl group, a substituted or unsubstituted C6 to C12 aryl group, a substituted or unsubstituted C2 to C12 heteroaryl group, or a combination thereof,
L³ is a substituted or unsubstituted phenylene group, a substituted or unsubstituted biphenylene group or a substituted or unsubstituted terphenylene group.

8. The composition for an organic optoelectric device of claim 1, wherein Z¹ to Z³ are independently CR^{b} or N,
at least one of Z¹ to Z³ is N,
R⁵ to R⁸ are independently hydrogen, or a substituted or unsubstituted phenyl group, and
R⁹, R¹⁰, and R^{b} are independently a substituted or unsubstituted phenyl group, a substituted or unsubstituted biphenyl group, a substituted or unsubstituted terphenyl group, a substituted or unsubstituted pyridinyl group, a substituted or unsubstituted pyridinyl group, a substituted or unsubstituted pyrimidinyl group, or a substituted or unsubstituted triazinyl group.

9. The composition for an organic optoelectric device of claim 8, wherein R⁹, R¹⁰ and R^{b} are independently one of substituents of Group II:
[Group II] 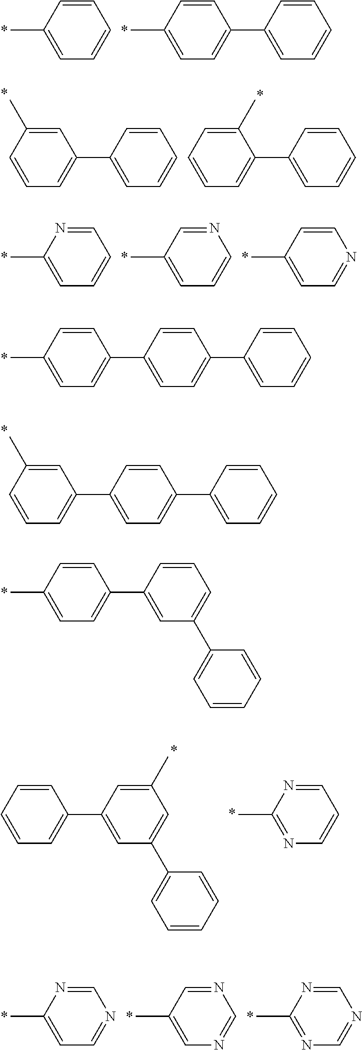
wherein, in Group II, * is a linking point.

10. The composition for an organic optoelectric device of claim 1, wherein 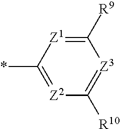
of Chemical Formula 3 is one of substituents of Group III:
[Group III] 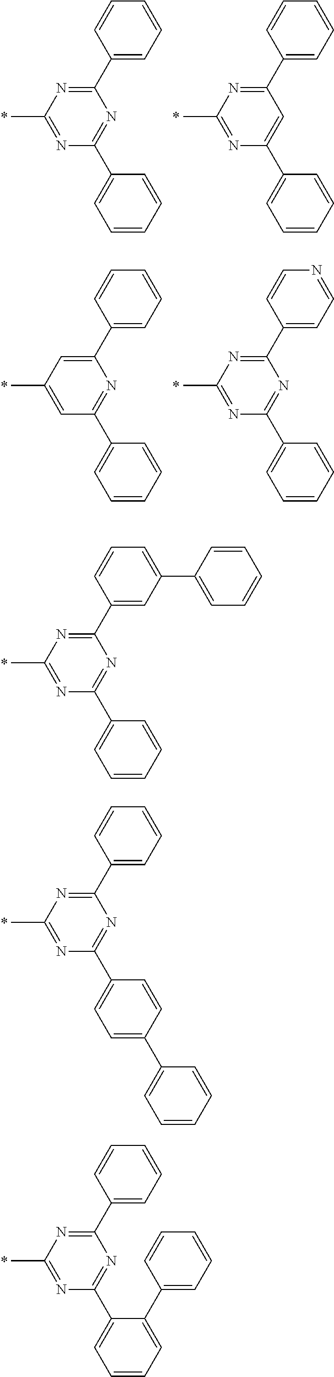 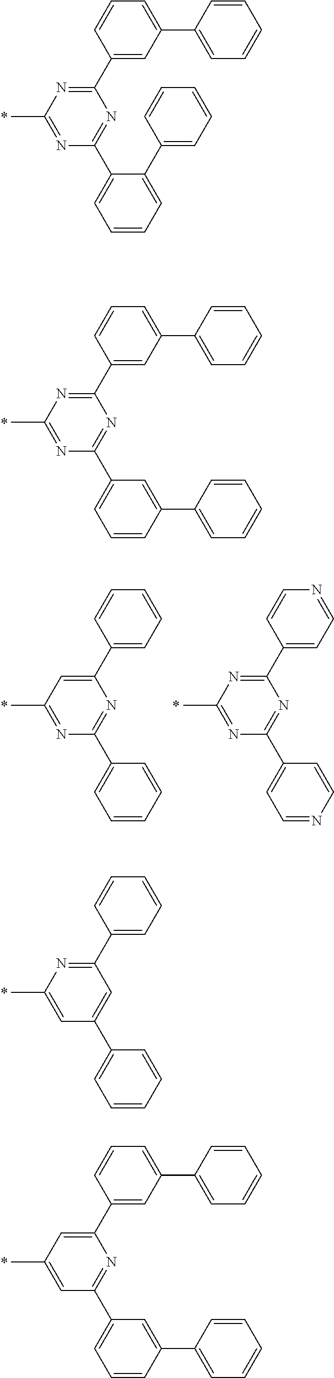 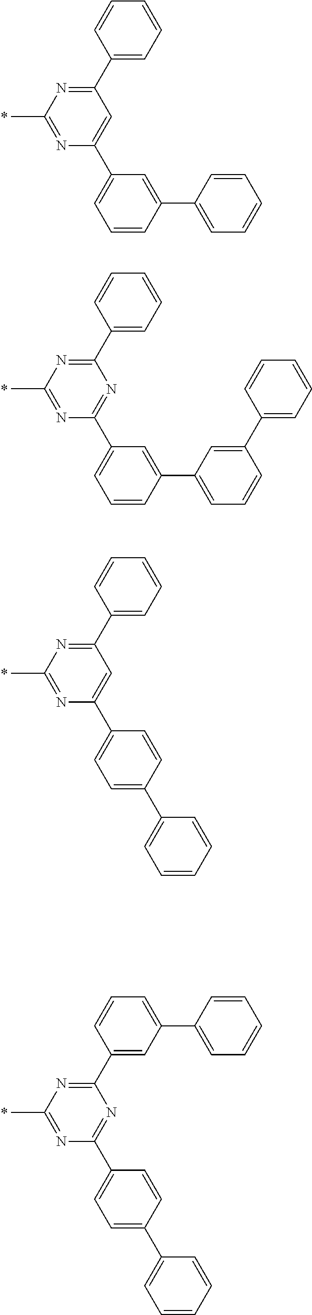 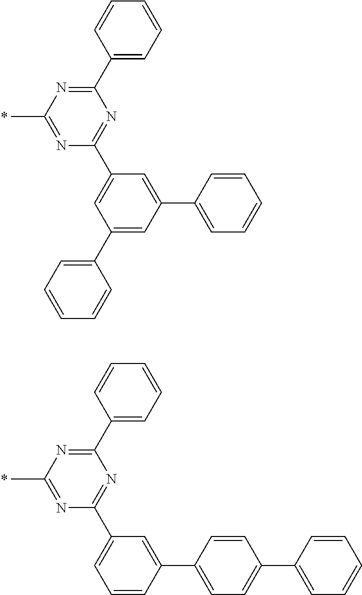
wherein, in Group III, * is a linking point.

11. The composition for an organic optoelectric device of claim 1, wherein the second host compound is one of compounds of Group 2:
[Group 2]

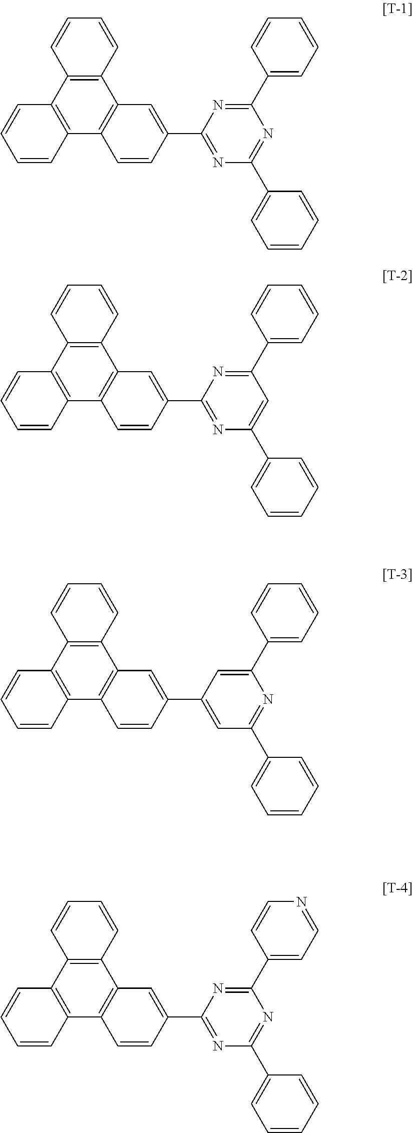

12. The composition for an organic optoelectric device of claim 1, wherein the first host compound is represented by Chemical Formula 1-C1 or Chemical Formula 1-E1, and the second host compound is represented by Chemical Formula 3-I: 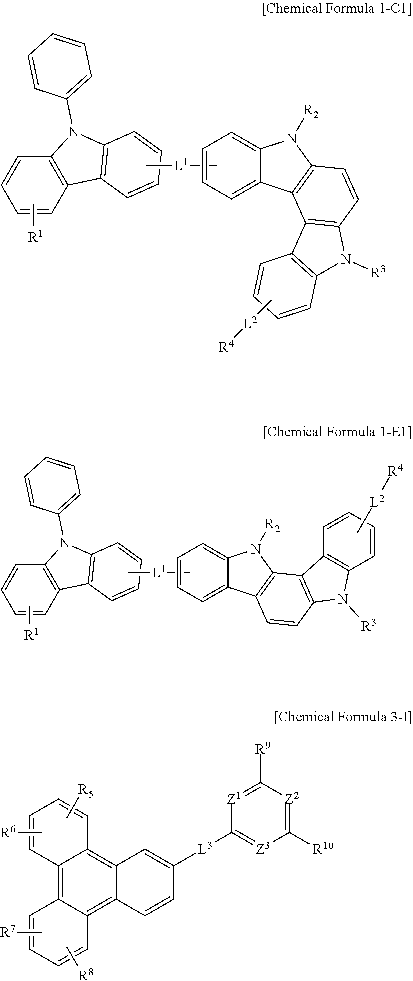
wherein, in Chemical Formulae 1-C1, 1-E1 and 3-I,
R¹ and R⁴ are independently hydrogen, deuterium, a substituted or unsubstituted C6 to C18 aryl group, or a combination thereof,
R² and R³ are independently a substituted or unsubstituted C6 to C18 aryl group,
L¹ and L² are independently a single bond, or a substituted or unsubstituted phenylene group,
Z¹ to Z³ are independently CR^{b} or N,
at least one of Z¹ to Z³ is N,
R⁵ to R¹⁰ and R^{b} are independently hydrogen, deuterium, a substituted or unsubstituted C1 to C10 alkyl group, a substituted or unsubstituted C6 to C12 aryl group, a substituted or unsubstituted C2 to C12 heteroaryl group, or a combination thereof, and
L³ is a substituted or unsubstituted phenylene group, a substituted or unsubstituted biphenylene group or a substituted or unsubstituted terphenylene group.

13. The composition for an organic optoelectric device of claim 1, wherein the composition further includes a phosphorescent dopant.

14. An organic optoelectric device comprising
an anode and a cathode facing each other, and
at least one organic layer between the anode and the cathode,
wherein the organic layer includes the composition for an organic optoelectric device of claim 1.

15. The organic optoelectric device of claim 14, wherein the organic layer includes an emission layer, and
the emission layer includes the composition for an organic optoelectric device.

16. A display device comprising the organic optoelectric device of claim 14.
